(19) United States
(12) Patent Application Publication
(10) Pub. No.: US 2020/0000840 A1
(43) Pub. Date: Jan. 2, 2020

(54) USE OF CYCLODEXTRINS TO REDUCE ENDOCYTOSIS IN MALIGNANT AND NEURODEGENERATIVE DISORDERS

(71) Applicant: Asdera LLC, New York, NY (US)

(72) Inventor: Knut M. Wittkowski, New York, NY (US)

(21) Appl. No.: 16/086,946

(22) PCT Filed: Mar. 20, 2017
(86) PCT No.: PCT/IB2017/000373

§ 371 Date: Sep. 20, 2018

Related U.S. Application Data
(60) Provisional application No. 62/310,774, filed on Mar. 20, 2016.

(51) Int. Cl. A61K 31/724; A61P 35/04; A61P 25/28; A61P 25/16

(52) U.S. Cl. CPC A61K 31/724; A61P 25/16; A61P 25/28; A61P 35/04

(57) ABSTRACT

The present disclosure provides certain compositions and methods that may be useful in the treatment and/or prevention of a malignant or neurodegenerative disease or disorder, such as cancer or Alzheimer's disease. Compositions are provided that contain at least one cyclodextrin active agent, such as α-cyclodextrin, or an analogue or derivative thereof.

### CROSS-REFERENCE TO RELATED APPLICATIONS

This application is a 35 U.S.C. § 371 national stage filing of International Application No. PCT/IB2017/000373, filed on Mar. 20, 2017, which claims priority to U.S. Provisional Application No. 62/310,774, filed on Mar. 20, 2016. The contents of each of the aforementioned applications are incorporated by reference in their entirety.

### BACKGROUND

2-Hydroxypropyl-β-cyclodextrin (HP-β-CD) is “generally recognized as safe” (GRAS) as food additives and frequently used as excipients with parenteral use to form water soluble compounds with lipophilic drugs. Parenteral and intrathecal HP-β-CD has been approved for the treatment of Niemann-Pick disease, for its potential to extract excess cholesterol, a lipid molecule, from lysosomes (Vance et al. 2014). As cholesterols is implicated in many other processes, including cell growth, HP-β-CD has also been suggested as a cancer treatment (Mohammad et al. 2014; Yokoo et al. 2015). Other proposed uses are neurodegenerative diseases, such as neural ceroid lipofuscinoses (Song et al. 2014), Parkinson's disease (PD), and Alzheimer's disease (AD), stroke, and brain tumors (Vecsernyes et al. 2014), “although no direct causal relationship [with] cholesterol metabolism” (Vance 2012). High levels of endocytosis have also been suggested as a mechanism underlying malignant and neurodegenerative processes. Aside from the role of endocytosis in cancer (Mellman et al. 2013), “inhibition of endocytosis reduces APP internalization and immediately lowers Aβ levels in vivo” (van Spronsen et al. 2010). While beneficial in early life, high levels of endocytosis may become detrimental with age. “Several early endosomal proteins have [already] been found to be causative of neurodegenerative disorders when mutated [because they] lead to the accumulation of undegraded macromolecules, . . . which are toxic to the cell” (Schreij et al. 2015). In support for a common mechanism for neurodegenerative diseases and cancer, amyloid precursor protein, the hallmark of AD, has been linked to cancer progression and metastasis (Pandey et al. 2016), as well as breast cancer (Lim et al. 2014), yet effective modulators of endocytosis are missing.

Metastases are the most lethal aspects of malignant disorders. Breast cancer, for instance, is the most common non-cutaneous malignancy in women. In 2015, 231,840 U.S. women were newly diagnosed and an estimated 3.1 million breast cancer survivors are alive in the U.S. (Runowicz et al. 2016). After lung cancer, it was the second most deadly, causing 40,290 deaths, 17,010 before the age of 65. “[W]omen who have one first-degree relative (mother, sister, or daughter) with a history of breast cancer are about twice as likely to develop breast cancer as women who do not have this family history” (American Cancer Society 2015), implicating that a substantial portion of cancer risk is inherited, yet the known complexes of DNA damage repair genes BRCA1/BRCA2/PALB2 and MRE11A/RAD50/NBN/RINT1 as well as PIK3CA/PTEN, which is currently believed to be mainly involved in growth, explain only 10% of the incidence. Prostate cancer, in turn, is the most common cancer in men. In 2015, 220.800 U.S. men were newly diagnosed and 27,540 died. Many epithelial cancers (derived from endodermal or ectodermal tissue, including, but not limited to breast, prostate, lung, pancreas, and colon carcinoma) are known to share risk factors. For instance, “[b]oth [breast and prostate] require gonadal steroids for their development, and tum[o]rs that arise from them are typically hormone-dependent and have remarkable underlying biological similarities” (Risbridger et al. 2010). Prostate cancer is known to be genetically linked with breast cancer. “Prostate cancer diagnosed among first-degree family members increases a woman's risk of developing breast cancer” (Beebe-Dimmer et al. 2015). AR and BRCA2 are among the many genes affiliated with both forms of cancer (lacopetta et al. 2012; Castro et al. 2012). Most importantly from the perspective of developing treatments with high benefit/risk ratio, neither breast nor prostate cancer are lethal, unless the spread to other organs, such as bones, liver, lungs, and brain. Hence, preventing metastases alone, rather than tumor growth, could significantly reduce the disease burden.

Currently, about 5 million U.S. individuals have Alzheimer's disease and about 1 million has Parkinson's disease. As life expectancy increases, the prevalence of neurodegenerative diseases or disorders also increases. Ten percent of people age 65 and older and 15% of people age 75 or older have Alzheimer's disease; almost two thirds of Americans with Alzheimer's are women; African Americans and Hispanics are more likely to develop Alzheimer's than Caucasians.

Treatments of a malignant or neurodegenerative disease or disorders generally include local therapy (for instance: surgery with or without radiation in breast cancer, surgery or radiation in prostate cancer) and adjuvant systemic therapy (hormonal therapy, chemotherapy, and biologic agents) for cancer cells that may have spread. Radiation and chemotherapy often cause substantial side-effects including, but not limited to nausea and hair loss. Hormone therapy for prostate cancer includes anti-androgens. For some hormone-receptor-positive forms of breast cancer, selective estrogen receptor modulators (SERM), such as tamoxifen and raloxifene, and aromatase inhibitors, such as exemestane and anastrozole, can interfere with disease progression. Monoclonal antibodies, such as trastuzumab and pertuzumab, are approved for the treatment of HER2 positive cancer. For patients with triple-negative (absence of estrogen, progesterone, and the Her2/neu receptor), treatment options are limited.

“Results with broadly acting [cyclin-dependent kinase (CDK)] inhibitors were largely disappointing” (Finn et al. 2016) and only a small number of patients benefit from phosphoinositide 3-kinase (PI3K) inhibitors (Bosch et al. 2015). Recently, HP-β-CD has been proposed for the treatment of cancers (Mohammad et al. 2014; Yokoo et al. 2015), but extracting cholesterols not only from cancer, but also from outer hair cell, may cause permanent hearing loss (Takahashi et al. 2016; Cronin et al. 2015).

There is an urgent need for a better understanding of the genetic risk factors for malignant diseases or disorders, including, but not limited to, metastases, in general, and the risk factors for metastatic breast or prostate cancer, in particular. Such findings and genetic analyses can be useful for defining drug targets, and developing therapeutic compounds and treatment methods which target the mechanisms underlying cell migration and tissue invasion, so that, instead of broadly attacking cell growth with radiation and cytotoxic drugs, therapeutic approaches can be developed to focus on the more deadly consequences of breast or prostate cancer, which are not the local cancer, but migration of cancer cells and invasion into other tissues, including bones.

Patients with Parkinson's disease initially benefit from treatment of motor symptoms (levodopa), but become non-responsive over time. Patients with Alzheimer's disease have few treatment options. Three recent phase 3 studies of BACE1 inhibitors have failed.

As “derailed endocytosis” has been linked to cancers (Mosesson et al. 2008; Mitra et al. 2012), neurodegenerative diseases (Van Dooren et al. 2014), and other “pathological conditions” (Di Fiore et al. 2014), the same treatment that preventing metastases in cancer might also prevent accumulation of undegraded macromolecules in neurodegenerative diseases and other many other age-related conditions. Yet, effective treatments to regulate endocytosis are lacking.

### SUMMARY OF THE INVENTION

Some of the main aspects of the present invention are summarized below. Additional aspects are described below in the Detailed Description of the Invention and Examples sections of the application. The description in each of the sections of this patent application is intended to be read in conjunction with the other sections. Furthermore, the various embodiments described in each of the sections of this patent application can combined in various different ways, and all such combinations are intended to fall within the scope of the present invention.

In one aspect, the present invention provides a method of treating a malignant or neurodegenerative disease or disorder in a subject, the method comprising administering to the subject an effective amount of a cyclodextrin, or an analogue or derivative thereof, either alone or in combination with one or more additional active agents. For example, in one embodiment, the present invention provides a method of treating a malignant or neurodegenerative disease or disorder in a subject, the method comprising administering to the subject an effective amount of the cyclodextrin α-cyclodextrin (αCD), or an analogue or derivative thereof, either alone or in combination with one or more additional active agents. In another aspect, the invention provides a composition comprising α-cyclodextrin, or an analogue or derivative thereof, for use in the treatment of an epithelial cancer (carcinoma) or the treatment of Parkinson's, Alzheimer's, or Huntington's disease. In another aspect, the derivative is a hydroxypropyl (HP) derivative.

In one aspect, the invention provides a method of improving one or more indicators of a malignant or neurodegenerative disease or disorder in a subject, the method comprising administering to a subject exhibiting one or more indicators of a malignant or neurodegenerative disease or disorder, an effective amount of α-cyclodextrin, or an analogue or derivative thereof, either alone or in combination with one or more additional active agents. For example, in one embodiment, the present invention provides a method of treating a malignant or neurodegenerative disease or disorder in a subject, the method comprising administering to the subject an effective amount of α-cyclodextrin, or an analogue or derivative thereof, either alone or in combination with one or more additional active agents.

In one aspect, the present invention provides a method of improving one or more indicators or symptoms of a malignant or neurodegenerative disease or disorder in a subject, the method comprising administering to a subject exhibiting one or more indicators or symptoms of a malignant or neurodegenerative disease or disorder, an effective amount of a cyclodextrin, or an analogue or derivative thereof, either alone or in combination with one or more additional active agents, wherein the indicator is selected from the group comprising results including, but not limited to, results of a blood test (including, but not limited to circulating tumor DNA and prostate-specific antigen), an x-ray evaluation, the result of a physical examination (including, but not limited to a palpable tumor), a psychiatric evaluation, or a tissue biopsy for histological evaluation and/or determination of hormone receptor status. In one embodiment, the “improving” comprises an increase of at least 1% in a measurement of the one or more indicators or symptoms. For example, in one embodiment, the present invention provides a method of improving one or more indicators or symptoms of a malignant or neurodegenerative disease or disorder in a subject, the method comprising administering to a subject exhibiting one or more indicators or symptoms of a malignant or neurodegenerative disease or disorder an effective amount of cyclodextrin, or an analogue or derivative thereof, either alone or in combination with one or more additional active agents, wherein a symptom or indicator is selected from the group comprising survival, disease-free survival, distant metastasis-free survival, results of a blood test (including, but not limited to circulating tumor DNA and prostate-specific antigen), an x-ray evaluation, the result of a physical examination (including, but not limited to a palpable tumor), or a tissue biopsy for histological evaluation. In some embodiments the cyclodextrin is α-cyclodextrin. In some embodiments the derivative is an hydroxypropyl derivative.

In some embodiments, the cyclodextrin is administered to the subject at a dose of about 2500 mg/kg α-cyclodextrin bi-weekly (about 700 mg/kg/d), the same dose used for HP-β-cyclodextrin in two children with NPC for “over a year, with no discernable side effects” for a “targeted concentration of 0.1-1.0 mM.” (INDs 104,114 and 104,116, approval date: 2009-04-13) In other embodiments, the dose will be adjusted over time to the highest dose not causing renal or hemolysis in the patient. In some embodiments, the cyclodextrin, such as α-cyclodextrin, is administered to the subject at a dose of at least 100 mg, at least 200 mg, at least 500 mg, at least 1000 mg, at least 2000 mg, at least 5000 mg, or at least 10,000 mg. In some embodiments, the cyclodextrin, such as α-cyclodextrin, is administered to the subject at a dose in the range of from about 1 to about 10,000 mg, from about 1 to about 7,500 mg, from about 1 to about 5,000 mg, from about 1 to about 2,500 mg, from about 1 to about 1,000 mg, from about 1 to about 500 mg, from about 1 to about 200 mg, from about 200 to about 10,000 mg, from about 200 to about 4,000 mg, from about 200 mg to about 2,000 mg, from about 200 to about 1,000 mg, or from about 200 to about 500 mg per day. In some such embodiments, each of the dosages described above is mg/kg/day. Additional dosages that may be used are provided in the Detailed Description section of this patent application.

In some embodiments the present invention provides a method of treating a malignant or neurodegenerative disease or disorder in a subject, the method comprising administering to the subject an effective amount of a drug reducing extracellular phospholipid. In some embodiment the a malignant or neurodegenerative disease or disorder will be an epithelial cancer (carcinoma). In some embodiment the a malignant or neurodegenerative disease or disorder will be breast cancer. In another embodiment the a malignant or neurodegenerative disease or disorder will be Alzheimer's disease. In some embodiment the a malignant or neurodegenerative disease or disorder will be Parkinson's disease. In some embodiment the a malignant or neurodegenerative disease or disorder will be Huntington's disease.

In some embodiments, the present invention provides various combinations of treatments, including pharmaceutical compositions. In some embodiments, cyclodextrins are used in combination with established pharmaceutical, radiological, or surgical interventions comprising cytotoxic interventions, receptor antagonists, monoclonal antibodies, radiation therapy, removal of tumor tissue, and the like.

In one embodiment, the subject is a human. In another embodiment the subject is an adult human.

### BRIEF DESCRIPTION OF THE DRAWINGS

FIG. 1: QR-Plot of ssGWAS results for CGEM. The “null” projection (horizontal) ends at the median among the endpoints of the convex projections for individual chromosomes. Genes to the right of the vertical line are above the cut-off for study-specific genome-wide significance. Regions below (to the left) of the cut-off are given for descriptive purposes only.

FIG. 2: QR-Plot of muGWAS results for CGEM. (see FIG. 1 for legend)

FIG. 3: QR-Plot of ssGWAS results for EPIC. (see FIG. 1 for legend)

FIG. 4: QR-Plot of muGWAS results for EPIC. (see FIG. 1 for legend)

FIG. 5: QR-Plot of ssGWAS results for PBCS. (see FIG. 1 for legend)

FIG. 6: QR-Plot of muGWAS results for PBCS. (see FIG. 1 for legend)

FIG. 7: PI cycle. Phosphatidyl-inositol (PI) is synthesized from myo-inositol (imported by HMIT) and PA (via CDP-DAG) which can be synthesized from lysophosphatic acid (LPA), PC, or PS, or salvaged from IP3 and DAG. It can also be synthesized from 1-acyl GPI. Arrows: PIPs are phosphorylated at a 3-, 4-, or 5- position by PI-kinases (left to right) and hydrolyzed by a plethora of phosphatases (right-to-left). Genes associated with BC in this GWAS are highlighted as inverted (bold: aGWS). Wide arrows in the center indicate the sequence of PIs involved in EEC. Hexagons: PI/PIPs; PM: plasma membrane, CCV: clathrin-coated vesicle, UCV: uncoated vesicle, EE: early endosome, LE: late endosome; LY: lysosome. Inverted gene names indicate genes associated with phosphatidylinositol signaling and/or endocytosis. Bold arrows indicate PIs associated with endocytosis.

FIG. 8A-FIG. 8B: Known relationship of genes implicated in muGWAS with stages in the process of endocytosis (FIG. 8A) and exocytosis (FIG. 8B). Boxes: genes identified in the present invention by stage of EEC: Formation of clathrin-coated vesicles and E3 ubiquitination, separation of inactive integrin (fast recycling) from active integrins (slow recycling), sorting between secretory, lysosomal, and (slow) recycling pathway, and lysosomal degradation. Underlined genes are known BC promoters and suppressors, respectively. Clathrin-mediated endocytosis (CME) begins with co-assembly of the heterotetrameric clathrin adaptor complex AP-2 with clathrin at PI(4,5)P₂-rich plasma membrane sites. AP-2 in its open conformation recruits clathrin and a number of additional endocytic proteins, many of which also bind to PI(4,5)P₂. CCP maturation may be accompanied by SHIP-2-mediated dephosphorylation of PI(4,5)P₂ to PI(4)P. Synthesis of PI(3,4)P₂ is required for assembly of the PX-BAR domain protein SNX9 at constricting CCPs and may occur in parallel with PI(4,5)P₂ hydrolysis to PI(4)P via synaptojanin, thereby facilitating auxilin-dependent vesicle uncoating by the clathrin-dependent recruitment and activation of PI3KC2α, a class II PI3-kinase. PI(3,4)P₂ may finally be converted to PI(3)P en route to endosomes by the 4-phosphatases INPP4A/B, effectors of the endosomal GTPase Rab5. Adapted from (Posor et al. 2015). In the early endosome, β1 integrins are sorted. Inactive integrins undergo fast “short loop” recycling; active integrins go to the late endosome/multivesicular body for slow “long group” recycling (RAB11), lysosomal degeneration (unless rescued by RAB25/CLIC3), or secretion via the trans-Golgi-network (TGN) mediated by RAB9. Fast recycling of epidermal growth factor receptor drives proliferation (Tomas et al. 2014), so one would expect gain-of-function mutations in FIG. 8A. Lysosomal and synaptic vesicle exocytosis share many similarities. Endolysosome-localized PIPs may regulate lysosomal trafficking (Samie et al. 2014) (derived, in part from Kegg pathways hsa04144 and hsa04721). Adapted from (Schmid et al. 2013; Bohdanowicz et al. 2013; Hesketh et al. 2014; Mosesson et al. 2008).

FIG. 9: Endocytic mechanisms underlying tumor cell migration and invasion through tissue barriers. The diagram presents a motile cell, the advancing lamellipodium of which moves directionally (arrow). Focal adhesions (FAs) are schematically shown, and integrin heterodimers are present at these. Cell migration necessitates polarized endocytosis and trafficking of FA complexes. Integrin internalization is controlled by dynamin, which is activated by microtubules (not shown), and protein kinases, such as FAK and protein kinase Cα (PKCα). Both clathrin- and caveolin 1 (CAV1)-coated domains of the plasma membrane are involved in internalization of integrin. Once in early endosome (EE), integrins may be sorted for degradation in lysosomes, recycled to the plasma membrane through a RAB4-dependent route, or transported to the perinuclear recycling compartment (PNRC). Recycling from the PNRC requires Rab11 family members, such as RAB25, and for some integrin heterodimers, also the protein kinase B (PKB)—GSK3β (glycogen synthase kinase-β) axis, ARF6 or certain isoforms of PKC. Human tumors often aberrantly express RAB25, display a specific repertoire of growth factor-induced integrin heterodimers or present abnormally stabilized microtubules, which promote trafficking of integrins. FAK, Integrin, RAB25, and PKB have functions associated with oncogenesis and/or display aberrant expression in human tumors. Modified from (Mosesson et al. 2008).

FIG. 10: Hydroxypropyl cyclodextrins. Up to n×3 degrees of substitution may be realized, with numerous positional and regioisomers possible. Substitutions for R include, but are not limited to, H (parent), methyl (including randomly methylated), butyl, 2-hydroxypropyl (HP), acetyl, succinyl, glucosyl, maltoseyl, carboxymethyl ether, phosphate ester, simple polymers, or carboxymethyl.

FIG. 11A-FIG. 11B: Structure and dimensions of cholesterol (FIG. 11A) and cyclodextrins (FIG. 11B). Typical cyclodextrins (CDs) contain 6, 7 or 8 D-glucose monomers in a ring, creating a cone shape that can accommodate guest molecules into their hydrophobic cavity. Cholesterol is too large to be completely encapsulated within the cavity of a single CD. So, cholesterol may be either partly incorporated into the cavity or two stacked CDs are required for the complete complexation of cholesterol. Modified from (Clear et al. 2015). Scheme 10.6 after (Gimpl et al. 2011).

FIG. 12: Clustering analysis of cholesterol interaction with α-CD and HPβCD. Displayed are the last 25 ns of the trajectories. CD molecules are depicted using the solid surface representation method with a probe radius of 0.4 Å, density isovalue of 0.7 and grid spacing of 0.5 to visualize the binding cavity. Hydrogen atoms are omitted to enhance clarity. Modified from (shityakov et al. 2016).

FIG. 13: HaCaT Proliferation. Influence of cyclodextrins on the proliferation of HaCaT keratinocytes. Mean values after 48 h incubation, normalized to the control, of at least six independent measurements. Top: ATP-Assay, bottom: PicoGreen-Assay. W6: α-CD, W6: β-CD, W7s HP: HP-β-CD. Modified from (Hipler et al. 2007).

FIG. 14: HaCaT Caspase 3/7 Activity. Influence of cyclodextrin on Caspase 3/7 activity of spontaneously immortalized aneuploid human (HaCaT) keratinocytes. Mean values after incubation (24 h), normalized on the control and the protein content, of at least six independent measurements. W6: α-CD, W6: β-CD, W7s HP: HP-β-CD. Modified from (Hipler U C, Schonfelder U, et al. (2007) J Blamed Mater Res A 83:70-9)

FIG. 15: HaCaT Lactose Dehydrogenase. Influence of cyclodextrin on lactose-dehydrogenase (LDH) of spontaneously immortalized aneuploid human (HaCaT) keratinocytes. Mean values after incubation (48 h), normalized on the control and the protein content, of at least six independent measurements. W6: α-CD, W6: β-CD, W7s HP: HP-β-CD. Modified from (Hipler et al. 2007).

FIG. 16A-FIG. 16C: Specificity of Cyclodextrin Release. Release of phospholipids (FIG. 16A), cholesterol (FIG. 16B) and proteins (FIG. 16C) from intact (FIG. 16B) or ghost (FIG. 16A and FIG. 16C) erythrocytes treated with cyclodextrins. (◯) α-Cyclodextrin; (●) β-cyclodextrin; (Δ) γ-cyclodextrin. Modified from: (Ohtani et al. 1989).

FIG. 17A-FIG. 17B: Specificity of Lipid Release II. FIG. 17A: Cholesterol released from brain capillary endothelial cell (BCECs) after 2 hours of incubation in the presence of various concentrations of α- and β-CDs. Results are expressed as a percentage of cholesterol released from BCECs compared with the control. Each percentage is the mean of three different filters and representative of two series of independent experiments. FIG. 17B: Phosphatidylcholine (light columns) and sphingomyelin (dark columns) released from BCECs after 2 h of incubation in the presence of α- and β-CDs at 5, 5, and 50 mM, respectively. Results are expressed as a percentage of phospholipids released from BCECs compared with the control. Each percentage is the mean of three different filters and representative of two series of independent experiments Modified from: (Monnaert et al. 2004).

FIG. 18: EEC in Alzheimer's disease (AD). APP is synthesized in the ER, transported to the TGN, and inserted into the plasma membrane via secretory vesicles. Cell-surface APP can be internalized to endosomes from which it can either be recycled back to the cell surface or delivered to lysosomes for degradation. Within the EE, the acidic environment favors production of Aβ, which can be degraded in lysosomes by cathepsins, accumulated in EEs, or released to extracellular spaces via exocytosis. (Modified from (Chen et al. 2014).

FIG. 19: EEC in Parkinson's disease (PD) Following endocytic entry, cargo is transported to early endosomes. From there, cargo can recycle back to the plasma membrane, either directly or via recycling endosomes. Alternatively, cargo can be retained in the EEs, which will form LEs/MVBs, and fuse with lysosomes for degradation. In parallel, cargo are transported between EEs and the trans-Golgi network (TGN). Alterations in these processes lead to dysfunctional lysosomes and accumulation of undegraded macromolecules, toxic to the cell (adopted from (Schreij et al. 2015)).

FIG. 20: Wound Healing Assay: (CBA-120, Cell BioLabs Inc.).

FIG. 21: Wound Healing Results. The dashed horizontal line indicates the reduction in wound closure by 1 mM HPaCD (see Table 7 for details).

FIG. 22: Genes identified by Study. Study; IPV6/IPV1: −log 10(p-value) in muGWAS/ssGWAS; Mbrn: membrane-associated genes (GPCR, Fc-Receptor, HA, RTK, Ion channels), PI/EC: phosphatidylinositol signaling/endocytosis, MPK: MAP kinases, Ncls: nucleus (cell cycle control, transcription, splicing). Top block of rows within each study: genes with diplotypes above the study-specific level of genome wide significance in muGWAS (CGEM: 5.29, FIG. 2; EPIC: 5.71, FIG. 4; PBCS: 5.13, FIG. 6); center block of rows within each study: other genes with diplotypes among the top 41 in muGWAS; bottom block of rows within each study: genes with SNPs above the study-specific level of genome-wide significance in ssGWAS (CGEM: 4.03, FIG. 1; EPIC: 4.00, FIG. 3; PBCS: 3.84, FIG. 5); ssGWAS results for genes also implicated in muGWAS are shown next to the muGWAS results); black squares: known breast cancer genes (from http://www.genecards.org); Boxed gene names indicate replication of the same gene (bold, PRKCQ, MEGF11) or replication of a closely related gene belonging to the same family (ATP8A1/ATP8B1, AGPAT3/AGPAT4, BMP7/BMPR1B).

### DETAILED DESCRIPTION OF THE INVENTION

The present invention is based, in part, on the discovery of certain disease-relevant collections of genes following a reanalysis of three independent sets of breast cancer genetic data, which are available for analysis from the National Institutes of Health's dbGaP collection. This reanalysis differed from the earlier analyses by using a novel computational biostatistics method (Wittkowski et al. 2014). As described in detail in the Examples, this patented method (Wittkowski et al. 2008; U.S. Pat. No. 7,664,616) addresses the following four points, which prior analyses of the same data using conventional bioinformatics approaches failed to consider: (i) non-additive relationships between risk alleles and incidence, (ii) cis-epistatic interaction, (iii) correlation between significance and minor allele frequency or “MAF” and (iv) non-randomization bias. It also addresses multiplicity adjustment for diplotype length, a problem arising from the use of a wide-locus approach. By addressing these points, the same strategy previously identified two novel collections of autism-specific genes (Wittkowski et al. 2014).

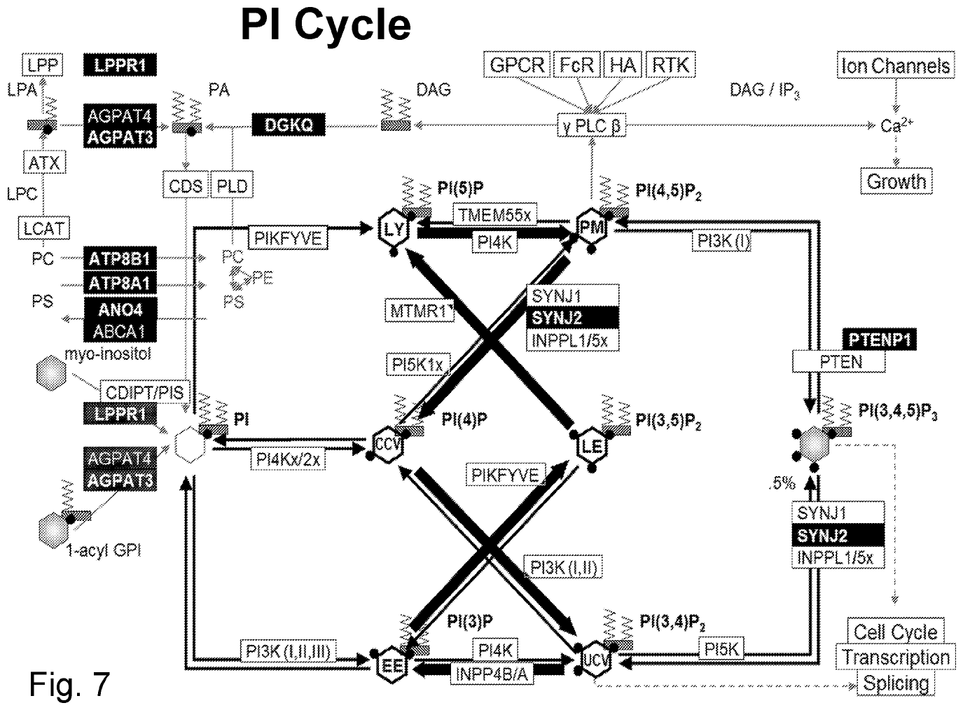

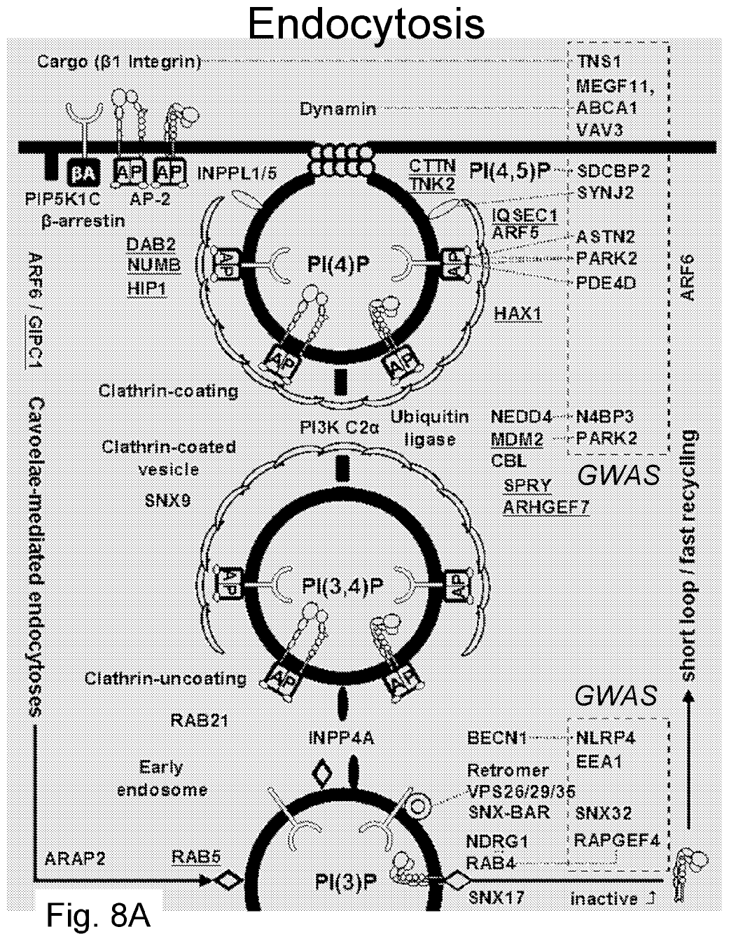

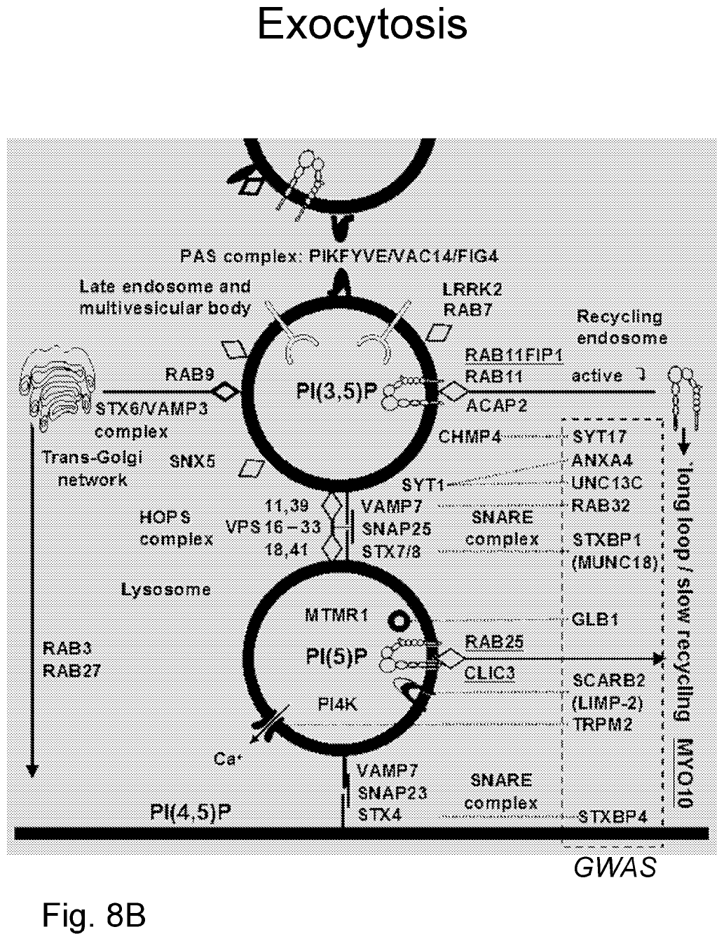

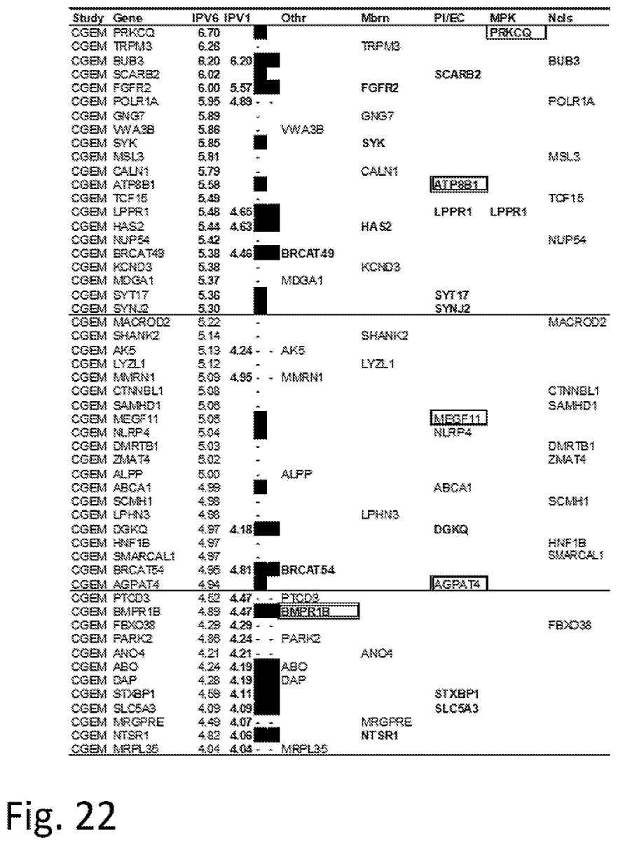

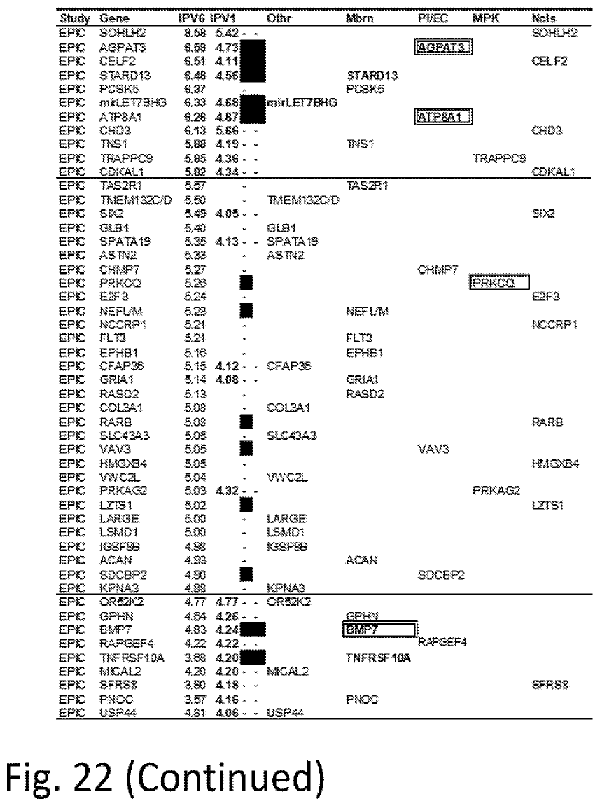

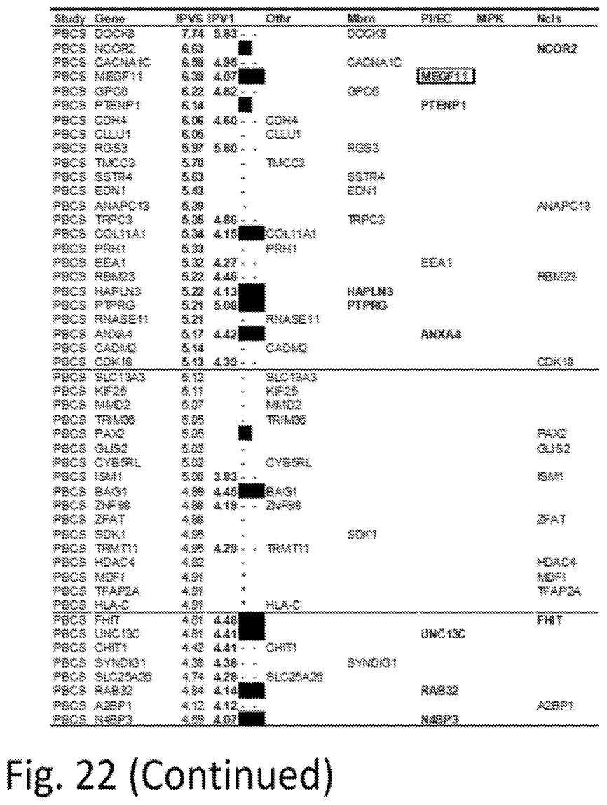

One collection of genes presented herein (FIG. 7, upper left corner) comprises eight genes whose roles include providing the phosphatidylinositol (PI) cycle with its substrate, PI. In AD, “PI is one of only 10 serum lipids that can accurately predict memory loss in as much as 90% of cases, 2 years before the onset of dementia symptoms” (Waugh 2015). The second gene cluster comprises genes directly associated with endocytosis, a process controlled by PI-phosphatases (PIPs) (FIG. 22, column PI/EC). The stages include, but are not limited to invagination and forming of clathrin-coated vesicles and early endosomes (FIG. 8). Based on this discovery, the present invention provides, in part, a shift in the focus for cancer treatments from controlling cell growth, a process involved in many vital functions even in older subjects, to a more to controlling migration and invasion of cells, a process of relevance primarily during prenatal and early postnatal development. Based on these findings, as presented in detail herein, in some embodiments the present invention provides compositions and methods for treating a malignant or neurodegenerative disease or disorder by modulating the PI cycle and its activity. Furthermore, in some embodiments, and based in part of the involvement of PIs, the present invention provides compositions and methods that may be useful for the treatment of a malignant or neurodegenerative disease or disorder during periods, such as adulthood, where cell migration, including neuronal growth, has mostly ceased, while cell growth, such as growth of hair, skin, and the like, continues, and where other cellular mechanisms may decline in an age-dependent manner.

In another part, the present invention is based on the reevaluation of interpretations of published findings, which are commonly believed to assert that α-cyclodextrin is as nephrotoxic as β-cyclodextrin, although nephrotoxicity was primarily demonstrated for β-cyclodextrin, which has substantially lower aqueous solubility than α-cyclodextrin, and, thus, a higher risk of forming the long cytoplasmic crystals seen in the kidneys of rats. In summary, “the [Joint Expert] Committee [on Food Additives (JECFA)] was reassured by the relatively low toxicity of this compound in animals and the fact that it was less toxic than beta-cyclodextrin, for which studies of human tolerance were available” (Prakasch et al. 2001).

In another embodiment, nephrotoxicity is further reduced by reducing the rate of delivery, the method comprising repeated doses per day (Frank et al. 1976), administering the drug over several hours via a peristaltic pump (Pitha et al. 1982), or administering the drug continuously via an implanted drug delivery system.

Some of the main embodiments of the present invention are described in the above Summary of the Invention section of this application, as well as in the Examples, Figures, and Claims. This Detailed Description of the Invention section provides additional description relating to the compositions and methods of the present invention, and is intended to be read in conjunction with all other sections of the present patent application, including the Summary of the Invention, Examples, Figures, and Claims sections of the present application.

### I. Abbreviations and Definitions

The abbreviation “αCD” refers to alpha-cyclodextrin.

The abbreviation “AD” refers to Alzheimer's disease.

The abbreviation “AKT” refers to protein kinase B.

The abbreviation “βCD” refers to beta-cyclodextrin.

The abbreviation “Ca” refers to calcium.

The abbreviation “CD” refers to cyclodextrin.

The abbreviation “CDK” refers to cyclin-dependent kinase.

The abbreviation “CGEM” refers to Cancer Genetic Markers of Susceptibility.

The abbreviation “Chr” refers to chromosome.

The abbreviation “dbGaP” refers to database of Genotypes and Phenotypes.

The abbreviation “DNA” refers to deoxyribonucleic acid

The abbreviation “EC” refers to endocytosis.

The abbreviation “EE” refers to early endosome.

The abbreviation “EPIC” refers to European Prospective Investigation into Cancer.

The abbreviation “ER” refers to endoplasmatic reticulum

The abbreviation “FAK” refers to focal adhesion kinase.

The abbreviation “Fc” refers to fragment, crystallizable

The abbreviation “γCD” refers to gamma-cyclodextrin.

The abbreviation “GPCR” refers to G-protein coupled receptor.

The abbreviation “GWAS” refers to genome-wide association study.

The abbreviation “GRAS” refers to generally recognized as safe.

The abbreviation “HA” refers to Hyaluronic acid

The abbreviation “HER2/neu” refers to receptor tyrosine-protein kinase erbB-2.

The abbreviation “HLA” refers to human leukocyte antigen.

The abbreviation “HP” refers to hydroxypropyl.

The abbreviation “HPαCD” refers to hydroxypropyl-alpha-cyclodextrin.

The abbreviation “HPaCD” refers to hydroxypropyl-alpha-cyclodextrin.

The abbreviation “HPβCD” refers to hydroxypropyl-beta-cyclodextrin.

The abbreviation “HPbCD” refers to hydroxypropyl-beta-cyclodextrin.

The abbreviation “IND” refers to investigational new drug.

The abbreviation “IPV” refers to inverse p-value.

The abbreviation “i.v.” refers to intravenous.

The abbreviation “LD” refers to linkage disequilibrium.

The abbreviation “LD₅₀” refers to median lethal dose.

The abbreviation “LE” refers to late endosome.

The abbreviation “MAF” refers to minor allele frequency.

The abbreviation “MAP refers to mitogen-activated protein.

The abbreviation “mTOR” refers to mechanistic target of rapamycin.

The abbreviation “muGWAS” refers to multivariate u-statistics GWAS.

The abbreviation “NIH” refers to National Institutes of Health.

The abbreviation “NPC” refers to Niemann Pick disease type C.

The abbreviation “PBCS” refers to Polish Breast Cancer Case-Control Study.

The abbreviation “PD” refers to Parkinson's disease.

The abbreviation “PI” refers to phosphatidylinositol.

The abbreviation “PIP” refers to phosphatidylinositol phosphate

The abbreviation “PIP” refers to PI(4)P.

The abbreviation “PIP2” refers to PI(4,5)P₂.

The abbreviation “PIP3” refers to PI(3,4,5)P₃.

The abbreviation “PM” refers to plasma membrane”

The abbreviation “PKB” refers to protein kinase B.

The abbreviation “PKC” refers to protein kinase C.

The abbreviation “QQ” refers to “quantile-quantile”.

The abbreviation “QR” refers to “quantile-rank”.

The abbreviation “RTK” refers to receptor tyrosine kinase.

The abbreviation “s.c.” refers to subcutaneous.

The abbreviation “SNP” refers to single nucleotide polymorphism.

The abbreviation “ssGWAS” refers to single-SNP genome-wide association study.

The abbreviation “TSC” refers to tuberous sclerosis.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of” “Consisting essentially of,” when used in the claims, shall have its ordinary meaning as used in the field of patent law.

The term “about” as used herein when referring to a measurable value such as an amount, a temporal duration, and the like, is meant to encompass variations of ±20%, ±10%, ±5%, ±1%, ±0.9%, ±0.8%, ±0.7%, ±0.6%, ±0.5%, ±0.4%, ±0.3%, ±0.2% or ±0.1% from the specified value, as such variations are appropriate to perform the disclosed methods.

As used herein, the terms “treat,” “treating,” and “treatment” encompass a variety of activities aiming at desirable changes in clinical outcomes. For example, the term “treat”, as used herein, encompasses any detectable improvement in one or more clinical indicators or symptom of a malignant or neurodegenerative disease or disorder—such as such as carcinomas, including, but not limited to, breast or prostate cancer, or neurodegenerative diseases, including, but not limited to, Parkinson's and Alzheimer's Disease. For example, such terms encompass alleviating, abating, ameliorating, relieving, reducing, inhibiting, preventing, or slowing at least one clinical indicator or symptom, preventing additional clinical indicators or symptoms, reducing or slowing the progression of one or more clinical indicators or symptoms, causing regression of one or more clinical indicators or symptoms, relieving a condition caused by the disease or disorder, and the like. As used herein the terms “treat,” “treating,” and “treatment” encompass both prophylactic treatments and therapeutic treatments. In the case of prophylactic treatments, the methods and compositions provided herein can be used preventatively in subjects that do not yet exhibit any clear or detectable clinical indicators or symptoms of the disease or disorder but that are believed to be at risk of developing the disease or disorder, such as a malignant or neurodegenerative disease or disorder. In the case of therapeutic treatments, the methods and compositions provided herein can be used in subjects that already exhibit one or more clinical indicators or symptoms of the disease or disorder, such as a malignant or neurodegenerative disease or disorder. In the case of a malignant or neurodegenerative disease or disorder, various clinical indicators and symptoms are known to medical practitioners and those of skill in the art.

The terms “prevent” or “preventing” as used herein encompasses stopping a disease, disorder, or symptom from starting, as well as reducing or slowing the progression or worsening of a disease or disorder. For example, “preventing” breast cancer or prostate cancer includes, but is not limited to, inhibiting the formation of cancerous cells or inhibiting the metastasis of malignant growths.

The term “a malignant or neurodegenerative disease or disorder” is used herein in accordance with it usual usage in the art and includes, but is not limited to malignant disorders, such as carcinomas, breast cancer, prostate cancer, malignancies of the breast, and malignancies of the prostate, as well as neurodegenerative diseases and disorders, such as Alzheimer's disease, Parkinson's disease, Huntington's disease, and “a range of disorders including brain overgrowth syndromes, [and] Charcot-Marie-Tooth neuropathies” (Waugh 2015).

As used herein, the term “cancer” or “hyperproliferative disease” is meant to refer to those diseases and disorders characterized by hyperproliferation of cells. Examples of hyperproliferative disease includes all forms of cancer, psoriasis, neoplasia, and hyperplasia.

The terms “subject,” “individual,” and “patient” are used interchangeably herein to refer to a vertebrate, preferably a mammal, more preferably a human. Mammals include, but are not limited to, murines, simians, humans, farm animals, cows, pigs, goats, sheep, horses, dogs, sport animals, and pets. Tissues, cells and their progeny obtained in vivo or cultured in vitro are also encompassed by the definition of the term “subject.” The term “subject” is also used throughout the specification in some embodiments to describe an animal from which a cell sample is taken or an animal to which a disclosed cell or nucleic acid sequences have been administered. In some embodiment, the animal is a human. For treatment of those conditions which are specific for a specific subject, such as a human being, the term “patient” may be interchangeably used. In some instances in the description of the present disclosure, the term “patient” will refer to human patients suffering from a particular disease or disorder. In some embodiments, the subject may be a non-human animal from which an endothelial cell sample is isolated or provided. The term “mammal” encompasses both humans and non-humans and includes but is not limited to humans, non-human primates, canines, felines, murines, bovines, equines, caprines, and porcines.

The terms “effective amount” or “therapeutically effective amount,” as used herein, refer to an amount of an active agent as described herein that is sufficient to achieve, or contribute towards achieving, one or more desirable clinical outcomes, such as those described in the “treatment” or “preventing” definition above. An appropriate “effective” amount in any individual case may be determined using standard techniques known in the art, such as a dose escalation study.

As used herein, the term “therapeutically effective amount” is meant to refer to an amount of an active agent or combination of agents effective to ameliorate or prevent the symptoms, shrink tumor size, prevent progression of cancer from non-metastatic to metastatic disease, or prolong the survival of the patient being treated for cancer or neurodegenerative disease. Determination of a therapeutically effective amount is well within the capabilities of those skilled in the art, especially in light of the detailed disclosure provided herein.

In some embodiments, cyclodextrin or derivatives thereof or pharmaceutically acceptable salts thereof can be co-administered with other therapeutics and/or part of a treatment regimen that includes radiation therapy.

The co-administration of therapeutics can be sequential in either order or simultaneous. In some embodiments cyclodextrin or derivatives thereof or pharmaceutically acceptable salts thereof is co-administered with more than one additional therapeutic. Examples of chemotherapeutics include common cytotoxic or cytostatic drugs such as for example: methotrexate (amethopterin), doxorubicin (adrimycin), daunorubicin, cytosinarabinoside, etoposide, 5-4 fluorouracil, melphalan, chlorambucil, and other nitrogen mustards (e.g. cyclophosphamide), cis-platin, vindesine (and other vinca alkaloids), mitomycin and bleomycin. Other chemotherapeutics include: purothionin (barley flour oligopeptide), macromomycin. 1,4-benzoquinone derivatives and trenimon. Anti-cancer antibodies, such as herceptin, and toxins are also examples of other additional therapeutics.

The therapeutic regimens can include sequential administration of cyclodextrin or derivatives thereof or pharmaceutically acceptable salts thereof and initiation of radiation therapy in either order or simultaneously. Those skilled in the art can readily formulate an appropriate radiotherapeutic regimen. Carlos A Perez & Luther W Brady: Principles and Practice of Radiation Oncology, 2nd Ed. JB Lippincott Co, Phila., 1992, which is incorporated herein by reference in its entirety describes radiation therapy protocols and parameters which can be used in the present invention.

When used in as part of the combination therapy the therapeutically effective amount of the inhibitor may be adjusted such that the amount is less than the dosage required to be effective if used without other therapeutic procedures.

In some embodiments, treatment with pharmaceutical compositions according to the invention is preceded by surgical intervention.

The disclosure also relates to methods of reducing the number of exosomes in a cancer cell by contacting said cancer cell with a therapeutically effective amount of a cyclodextrin.

The term “pharmaceutical composition” as used herein refers to a composition comprising at least one active agent as described herein (such as, for example, an α-cyclodextrin), and one or more other components suitable for use in pharmaceutical delivery such as carriers, stabilizers, diluents, dispersing agents, suspending agents, thickening agents, excipients, and the like.

The term “active agent” as used herein refers to a molecule that is intended to be used in the compositions and methods described herein and that is intended to be biologically active, for example for the purpose of treating a malignant or neurodegenerative disease or disorder. The term “active agent” is intended to include molecules that either are, or can be converted to a form that is, biologically active. For example, the term “active agent” includes pro-drugs and/or molecules that are inactive or lack the intended biological activity but that can be converted to a form that is active or has the intended biological activity.

As used herein, the term “sample” refers generally to a limited quantity of something which is intended to be similar to and represent a larger amount of that thing. In the present disclosure, a sample is a collection, swab, brushing, scraping, biopsy, removed tissue, or surgical resection that is to be tested for clinical indicators of a disease or disorder, such as a malignant or neurodegenerative disease or disorder. In some embodiments, samples are taken from a patient or subject that is believed to have a malignant or neurodegenerative disease or disorder. In some embodiments, a sample believed to contain clinical indicators of a disease or disorder, such as a malignant or neurodegenerative disease or disorder, is compared to a “control sample” that is known not to contain one or a plurality of clinical indicators of a disease or disorder, such as a malignant or neurodegenerative disease or disorder. In some embodiments, a sample believed to contain a clinical indicator of a disease or disorder, such as a malignant or neurodegenerative disease or disorder, is compared to a control sample that is known to not contain a clinical indicator of a disease or disorder, such as a malignant or neurodegenerative disease or disorder. In some embodiments, a sample believed to contain a clinical indicator of a disease or disorder, such as a malignant or neurodegenerative disease or disorder, is compared to a control sample that contains the same clinical indicators of a disease or disorder, such as a malignant or neurodegenerative disease or disorder.

The term “scavenge” as used herein means uptake or chemically combine with.

The term “salt” refers to acidic salts formed with inorganic and/or organic acids, as well as basic salts formed with inorganic and/or organic bases. Examples of these acids and bases are well known to those of ordinary skill in the art. Such acid addition salts will normally be pharmaceutically acceptable although salts of non-pharmaceutically acceptable acids may be of utility in the preparation and purification of the compound in question. Salts of the embodiments include those formed from hydrochloric, hydrobromic, sulphuric, phosphoric, citric, tartaric, lactic, pyruvic, acetic, succinic, fumaric, maleic, methanesulphonic and benzenesulphonic acids.

In some embodiments, salts of the compositions comprising one or may be formed by reacting the free base, or a salt, enantiomer or racemate thereof, with one or more equivalents of the appropriate acid. In some embodiments, pharmaceutical acceptable salts of the present invention refer to amino acids having at least one basic group or at least one basic radical. In some embodiments, pharmaceutical acceptable salts of the present invention comprise a free amino group, a free guanidino group, a pyrazinyl radical, or a pyridyl radical that forms acid addition salts. In some embodiments, the pharmaceutical acceptable salts of the present invention refer to amino acids that are acid addition salts of the subject compounds with (for example) inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxybenzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic acid, or aromatic sulfonic acids, for example benzene-, p-toluene- or naphthalene-2-sulfonic acid. When several basic groups are present mono- or poly-acid addition salts may be formed. The reaction may be carried out in a solvent or medium in which the salt is insoluble or in a solvent in which the salt is soluble, for example, water, dioxane, ethanol, tetrahydrofuran or diethyl ether, or a mixture of solvents, which may be removed in vacuo or by freeze drying. The reaction may also be a metathetical process or it may be carried out on an ion exchange resin. In some embodiments, the salts may be those that are physiologically tolerated by a patient. Salts according to the present invention may be found in their anhydrous form or as in hydrated crystalline form (i.e., complexed or crystallized with one or more molecules of water).

The term “soluble” or “water soluble” refers to solubility that is higher than 1/100,000 (mg/ml). The solubility of a substance, or solute, is the maximum mass of that substance that can be dissolved completely in a specified mass of the solvent, such as water. “Practically insoluble” or “insoluble,” on the other hand, refers to an aqueous solubility that is 1/10,000 (mg/ml) or less. Water soluble or soluble substances include, for example, polyethylene glycol. In some embodiments, the polypeptide of the claimed invention may be bound by polyethylene glycol to better solubilize the composition comprising the peptide.

As used herein, percent “homology” or “sequence identity” is determined by using the stand-alone executable BLAST engine program for blasting two sequences (b12seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety).

Additional definitions and abbreviations are provided elsewhere in this patent specification or are well known in the art.

### II. Additional Description

Malignant and neurodegenerative diseases or disorders are complex diseases involving many genes along common pathways. Based in part on the findings that the phosphoinositides PI(3,4,5)P₃ and PI(3,4)P₂ are upregulated in malignant and neurological diseases (Waugh 2015) and activate Akt signaling (Ooms et al. 2015; Ma et al. 2008; Majerus et al. 2009), PI signaling is widely believed to be involved in cancer growth and neurodegeneration. In addition, INPP4B is known as a suppressor of some cancers, but as a risk factor for others, including, but not limited to, breast and pancreatic cancer (Woolley et al. 2015; Chew et al. 2016). PI3K signaling has also be implied in aging, cognitive decline, and Alzheimer's (O'Neill 2013). Still, “clinical results with single-agent PI3K inhibitors have been modest to date” (Mayer et al. 2016), in part because traditional GWAS have largely failed to elucidate the precise mechanism by which PI signaling contributes to cancer and neurodegenerative diseases.

From the results presented herein, breast or prostate cancer as well as Alzheimer's and Parkinson's disease are characterized by hyperactivity within the phosphatidylinositol (PI) cycle facilitation migration of cancer cells and infiltration of tissues, such as bone, brain, lung, and liver. In some aspects the present invention provides methods of treatment of breast or prostate cancer and Alzheimer's or Parkinson's disease that comprise administering to a subject one or more active agents that target PIPs to elicit changes in endo-/exocytosis thereby causing a dampening or decrease of cellular migration and infiltration or processing of proteins, including, but not limited to, APP, tau, and alpha-synuclein. Such active agents may specifically target particular PIPs or may act non-specifically on several different classes or types of PIPs to elicit a broad reduction of activity. Furthermore, such active agents could be, for example, compounds or drugs that are already being used safely in humans for other indications and could be repurposed for use in the treatment of malignant or neurodegenerative diseases or disorders.

### A) Active Agents

As further described in the Examples and other sections of the present application, agents that can be used in the methods of the invention may reduce the concentration of phospholipids available to cells, including, but not limited to, neurons and tumor cells. In one embodiment, the agent is a cyclodextrin. In another embodiment, the cyclodextrin is α-cyclodextrin. In yet another embodiment, the agent is hydroxypropyl- (HP-) α-cyclodextrin.

Cyclodextrins are natural compounds formed during bacterial digestion of cellulose. They cannot be hydrolyzed by common amylases, but can be fermented by the intestinal microflora. They are poorly resorbed and are considered generally recognized as safe (GRAS) for oral administration. On Dec. 22, 2004, α-cyclodextrin was declared GRAS for “use in selected foods, except meat and poultry, for fiber supplementation, as a carrier or stabilizer for flavors (flavor adjuvant), as a carrier or stabilizer for colors, vitamins and fatty acids and to improve mouthfeel in beverages (GRN No. 155). An α-cyclodextrin monograph is included in the U.S. Pharmacopeia/National Formulary (USP/NF25), the European Pharmacopoeia (EP 6.0), and the Handbook of Pharmaceutical Excipients. Cyclodextrins are exempted from the requirement of a tolerance under 40 CFR 180.950 when used in or on various food commodities. (FR 70 128 28780 2005-07-06)

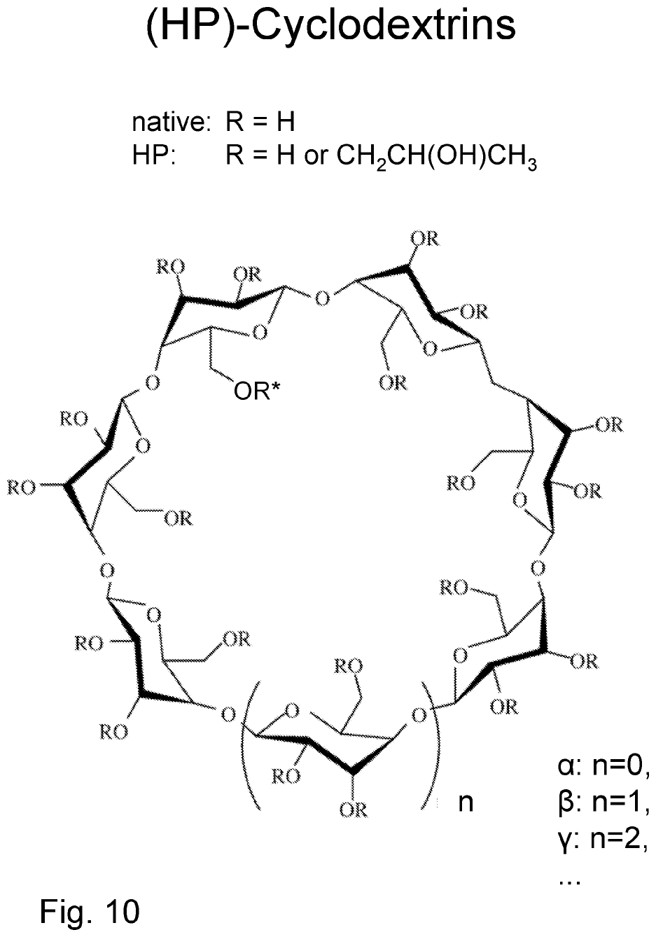

Cyclodextrins are compounds made up of (D-glucose) six (α-cyclodextrin), seven (β-cyclodextrin), or eight (γ-cyclodextrin) sugar molecules bound together in a toroid (truncated cone) with a lipophilic inner and a hydrophilic outer surface. (FIG. 10). This combination of features makes them suitable as an expedient to solubilize lipophilic drugs.

With parenteral delivery, “the steady-state volume of distribution for β-cyclodextrin . . . in rats, rabbits, dogs, and humans corresponds well with the extracellular fluid volume of each species, suggesting that no deep compartments or storage in pools are involved” (Irie et al. 1997). “α- and β-cyclodextrin are excreted almost completely in an intact form into the urine” (Irie et al. 1997).

Still, cyclodextrins may cause two types of adverse events with parenteral delivery. First, they can accumulate in kidney cells, causing nephrotoxicity. Second, after the lipophilic drug is delivered, they form “a new lipid-containing compartment (or pool) in the aqueous phase into which [cellular lipid] compounds [are] extracted” (Irie et al. 1997), which could cause hemolysis.

Nephrotoxicity of cyclodextrins was shown in 1976. In the rat, LD₅₀ for α- and β-CD was determined as 1008 and 788 mg/kg, respectively, the same minimal dose at which changes were observed by light microscopy from a single dose. “Repeated administration of nephrotoxic doses [of β-cyclodextrin] resulted in extensive nephrosis” (Frank et al. 1976). Nephrosis was not apparent in rats given 1, 2, 4, or 7 daily injections of 0.1 g/kg α-cyclodextrin; light microscopic lesions were found in one rat given 0.225 g/kg β-cyclodextrin daily for 4 days. Daily injections of 0.45 g/kg β-cyclodextrin resulted in severe nephrosis and produced no deaths. All rats given 0.9 g/kg β-cyclodextrin died within 4 days and [only] 1 died after 2 days of treatment with 1.0 g/kg α-cyclodextrin.” (Frank et al. 1976) (see Table 1 for details). In summary,

Only one rat died from daily doses of 1.000 α-cyclodextrin, while all rats died from daily doses of 0.900 β-cyclodextrin.

Electron microscopy was only conducted for β-cyclodextrin. Hence, it is not clear whether α-cyclodextrin also causes crystals and ultrastructure alterations or whether this outcome is a result of the lower solubility in water in β-cyclodextrin (18.5) vs α-cyclodextrin (145.0).

TABLE 1
Toxicity of cyclodextrins in rats

| | α-cyclodextrin [g/kg] | β-cyclodextrin [g/kg] |
|---|---|---|
| single dose i.v. LD₅₀ | 0.788/1.008 * | 0.788/1.008 * |
| nephrosis, | 0.100 no changes | 0.225 no changes |
| single dose s.c. | — | 0.450 no changes |
| | — | 0.670 changes seen |
| | 1.000 changes seen | 1.000 changes seen |
| nephrosis, | 0.100 no nephrosis | 0.225 one nephrosis after 4 days |
| 1, 2, 3, 4, or 7 daily doses s.c. | — | 0.450 severe nephrosis |
| | 1.000 severe nephrosis | 0.900 all died within 4 days |
| | one died after 2 days | |
| long cytoplasmic crystals | — | 0.980 |
| ultrastructure alterations | — | 0.450 |
| . . . 24 hours after injection | | increased number of vacuoles, |
| | | needle-like microcrystals in |
| | | lysosomes from nondegradable |
| | | CD |
| . . . 2-3 days after injection | — | extensive structural alterations |
| . . . 72 hours after injection | — | irreversible injury |

(Frank et al. 1976)
Legend:
“—” not done,
* the paper is ambiguous about which drug caused either LD₅₀, see also Ribeek, Prinsen as quoted in (http://www.inchem.org/documents/jecfa/jecmono/v48je10.htm)

Low solubility in water, as with β-cyclodextrin, in particular, often results in precipitation of solid cyclodextrin complexes. “In addition, β- and δ-cyclodextrin form intramolecular hydrogen bonds between secondary OH groups, which detracts from hydrogen bond formation with surrounding water molecules [resulting in] low aqueous solubilities” (Loftsson et al. 1996).

In four studies of outbred rodents (Riebeck 1990a/b/c, Prinsen 1991a, as quoted in WHO http://www.inchem.org/documents/jecfa/jecmono/v48je10.htm), macroscopic examination of dead and surviving animals either “did not reveal treatment-related alterations” (Riebeck 1990a/b/c) or “revealed a pale renal cortex” (Prinsen 1991a).

Although electron microscopy was performed only on β-cyclodextrin, but not in α-cyclodextrin, the abstract does not distinguish between the two cyclodextrins and, thus, α-cyclodextrin is now commonly believed to be at least as nephrotoxic as β-cyclodextrin:

“Early studies showing the nephrotoxicity of the parent CDs [Frank et al. 1976] . . . ” See p. 147 of (Irie et al. 1997).

“The renal toxicity of α-CD and β-CD after parenteral administration [Frank et al. 1976] . . . have been well documented [Irie et al. 1997; Thompson 1997 (review only); Gould et al. 2005 (β-CD only)].” See p. 31 of (Stella et al. 2008).

“Both α-CD and β-CD showed renal toxicity after parenteral administration.” See page 9 (EMA/CHMP/333892/2013).

“β-CD family (native, HPβCD and RAMEβ) was found [ . . . ] less toxic than α-CD family (native, HPαCD and RAMEα)[Monnaert et al. 2004]” (Coisne et al. 2016).

“α-CD is the most toxic among the three native CDs” (Monnaert et al. 2004).

However, when the dose “‘spike’ was spread to 6-8 hours . . . [d]oses of 1 g/kg not only did not result in animal deaths, but did not even influence the growth of the young animals compared to controls—a clear sign of lack of toxic effects” (Szejtli et al. 1981). Consistent with the latter results, “so far, however, there are no cases of kidney injury caused by cyclodextrins in humans” (EMA/CHMP/333892/2013; see also Table 3).

α-cyclodextrin is also well tolerated in cyclosporine eye-drops. (Kanai et al. 1989).

“Chemical modifications have been made to CDs to increase their hydrophilic activity with the hope that the improved solubility would eliminate the renal toxicity [in rats]” (Irie et al. 1997). “Substitution of any of the hydrogen bond forming hydroxyl groups, even by hydrophobic moieties such as methoxy and ethoxy functions, will result in a dramatic increase in water solubility . . . . The main reason for the solubility enhancement in these derivatives is that chemical manipulation frequently transforms the crystalline cyclodextrins into amorphous mixtures of isomeric derivatives” (Loftsson et al. 1996). “For example, the aqueous solubility of β-cyclodextrin . . . increases with increasing degree of methylation. The highest solubility is obtained when two-thirds of the hydroxyl groups (i.e., 14 of 21) are methylated” (Loftsson et al. 1996). 2-hydroxylpropyl-b-cyclodextrin (HP-b-CD), a hydroxyalkyl derivative, of β-CD, substantially improves water solubility (Table 1), the same dose of HP-β-cyclodextrin did not cause adverse clinical signs. (Gould et al. 2005).

Common cyclodextrins obtained by the substitution of the R groups on the edge of the α-cyclodextrin ring (FIG. 10; Brewster et al. 2007) include, but are not limited to,

methyl (including randomly methylated): CH₃,

2-hydroxypropyl (HP): CH₂CHOHCH₃,

Sulfobutylether: (CH₂)₄SO₃Na⁺

acetyl,

succinyl,

glucosyl,

maltoseyl,

carboxymethyl ether,

phosphate ester,

simple polymers, or

carboxymethyl.

“Since both the number of substitutes and their location will affect the physicochemical properties of the cyclodextrin molecules, such as their aqueous solubility and complexing abilities, each derivative listed should be regarded as a group of closely related cyclodextrin derivatives” (Loftsson et al. 1996).

TABLE 2
Characteristics of selected cyclodextrins

| | α- | HP-α- | β- | HP-β- |
|---|---|---|---|---|
| | cyclodextrin | cyclodextrin | cyclodextrin | cyclodextrin |
| Number of glucose units | 6 | 6 | 7 | 7 |
| Solubility in water [g/l] | 130-145 | ~500^{a} | 18.5 | >600 |
| Internal diameter | 4.7-5.2 | 4.7-5.2 | 6.0-6.4 | 6.0-6.4 |
| oral absorption in rats | 2-3% | | 0.6-2% | ≤3% |
| i.v. toxicity, 1 g daily | 1 died after 2 d | | all died within 4 d | |
| half-life (t_{1/2}) [h] | | | | 1.7-1.9 |
| Vol. of distribution (V_{D}) | | | | 0.2 |
| [l/kg] | | | | |
| Vₘₐₓ | 5.8 | | 166 | |
| acute i.v. toxicity in rats | 0.5-0.8 | | 1 | 10 |
| [g/kg] | | | | |
| BBB breakdown [mM] | 1 | 2.5 | 2.5 | 2.5 |
| Transport across BBB | 21.5 (0.5 mM)16.5 | 26.7 (1 mM) | 9.3 (1 mM) | |
| [%, 2 h] | (1 mM) | | | |
| max marketed dose i.v. | 1.3* | not suitable^{$} | 16,000 | |
| [mg/d] | | | | |

From (Brewster et al. 2007; Loftsson et al. 2010; Loftsson et al. 2016; Monnaert et al. 2004)
*Prostavasin 80 mg/d × 4 wk (NCT00596752) @ 649.3 μg CD per 20 μg alprostadil (caverjet label)
^{$}(Davis et al. 2004)
^{a}http://cyclolab.hu/index.php/standard-grade-cyclodextrins/hpacd

While 1 g/kg β-cyclodextrin caused severe nephrosis in rats (Table 1), the same dose of HP-β-cyclodextrin did not cause adverse clinical signs (Gould et al. 2005)
“8 hours after oral administration of 313 mg/kg ¹⁴C-βCD . . . 3 μg of βCD was detected in 1000 μg of blood.”

### Cytotoxicity/Hemolysis

Phospholipids and cholesterol, the major building blocks of cell membranes, are both lipids. Hence, when cyclodextrins are given intravenously without having their lipophilic cavity filled (or after the lipophilic drug has been delivered), cyclodextrins can potentially extract phospholipids and cholesterol from membranes. “Several CDs have been demonstrated to cause cell lysis in different types of cells, indicating that the effect is not cell-type specific [Irie et al. 1997]” (Stella et al. 2008).

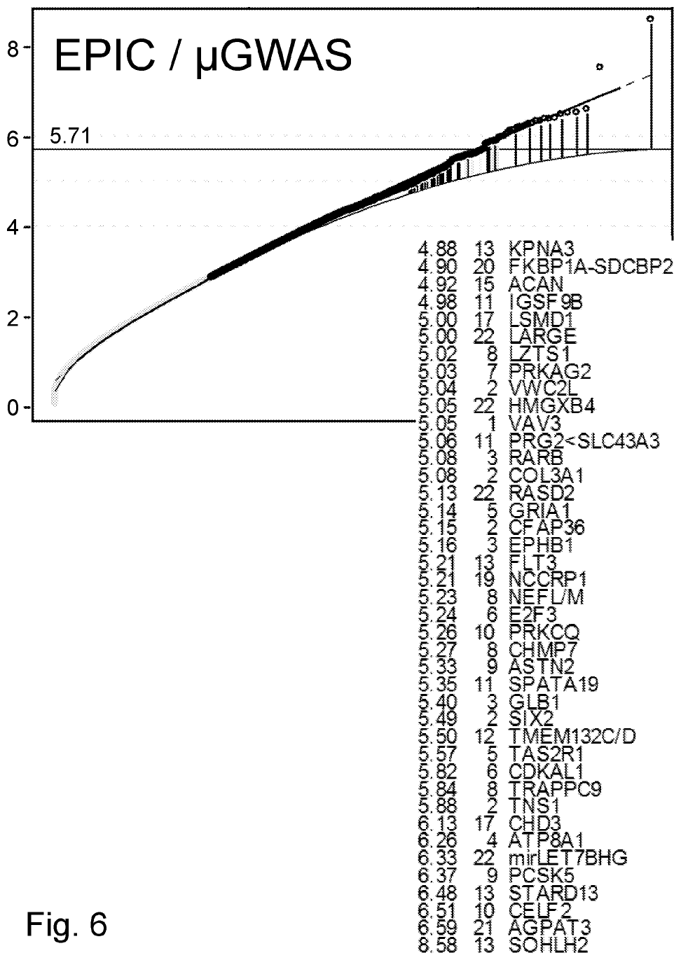

In particular, CDs are known to induce shape changes of membrane invagination on erythrocytes and, at higher concentrations, induce hemolysis of human erythrocytes in the order of β-CD>α-CD>γ-CD (Irie et al. 1982), i.e., the hemolytic activity of α-CD is lower than that of both β-CD and HP-β-CD. See FIG. 6 in (Irie et al. 1997). In addition, β-CD induces caspase-dependent apoptotic cell death in human keratinocytes on depletion of membrane cholesterol, whereas α-CD and HP-β-CD are not apoptotic to this type of cell (Stella et al. 2008).

Similarly, HP-α-cyclodextrin and maltosyl-α-cyclodextrin were found to be less cytotoxic than α-cyclodextrin on heterogeneous human epithelial colorectal adenocarcinoma (Caco-2) cells (Roka et al. 2015; Ono et al. 2001).

From these observations, HP-cyclodextrins not only have less nephrotoxicity, but also less cytotoxicity/hemolysis.

2-hydroxypropyl-beta-cyclodextrin (HP-β-CD) is used as an expedient/solvent for a many lipophilic drugs, including the neurosteroid allopregnanolol. The cyclodextrin α-cyclodextrin has been approved and is used as an expedient of alprostadil (a prostaglandin) for intracavernosal injection in the treatment of erectile dysfunction.

Still, less cytotoxicity overall may not be enough. A 2010 study showed that HP-β-CD causes permanent hearing loss in cats at doses of 4-8 g/kg, with raised concerns for the use of a cyclodextrin as a drug, rather than an expedient (smaller dose).

In 2014, it was observed that the reported benefit of the neurosteroid allopregnanolol in Niemann-Pick C (NPC) disease was, in fact, due to the solvent, 2-hydroxypropyl-beta-cyclodextrin (HP-β-cyclodextrin) (Vance et al. 2014). It was demonstrated the β-cyclodextrin at therapeutic doses extracts cholesterol from cellular components, rather than from cell membranes as previously believed. In September 2015, a phase 2b/3 study of 2-hydroxypropyl-beta-cyclodextrin started in patients with neurologic manifestations of Niemann-Pick Type C1 disease (NIH (2015) ClinicalTrialsgov NCT02534844). The question was whether one could retain the benefit for treating NPC while avoiding risk of ototoxicity (“dead or deaf?”).

There is a positive correlation between the hemolytic activity of several CDs and their capacity to solubilize cholesterol” (Irie et al. 1997), suggesting that ototoxicity might be directly related to the extraction of cholesterol from outer hair cells.

The results of the genetic study reported herein show that progression in breast cancer, including metastases are caused, in part, excessive the conversion of glycerophospholipids (LPC, PC, PS, PA) into PI, the substrate of the PI cycle, which regulates endocytosis by making phosphoinositides available.

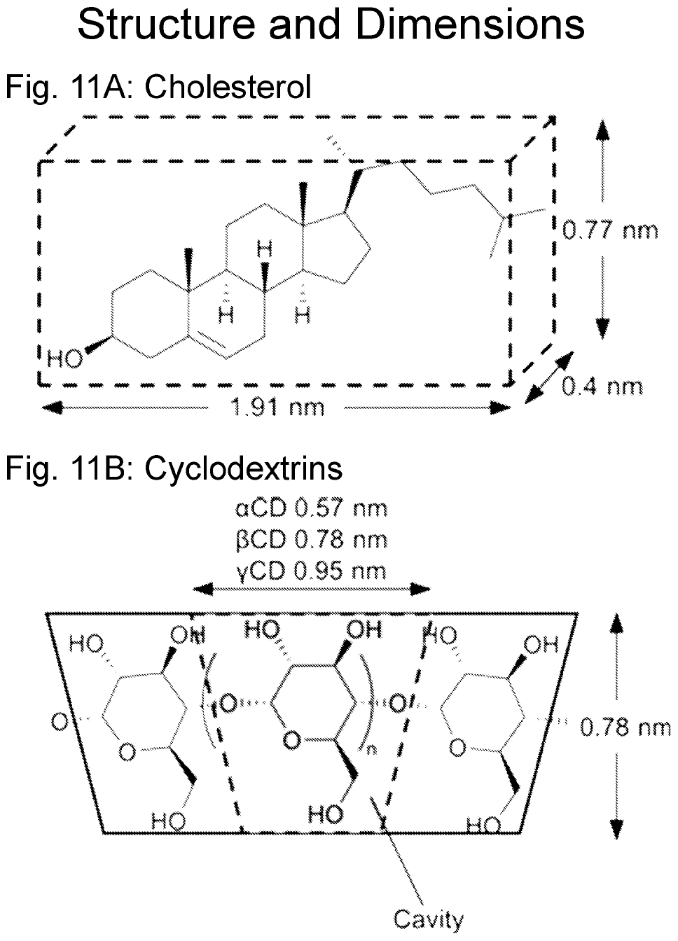

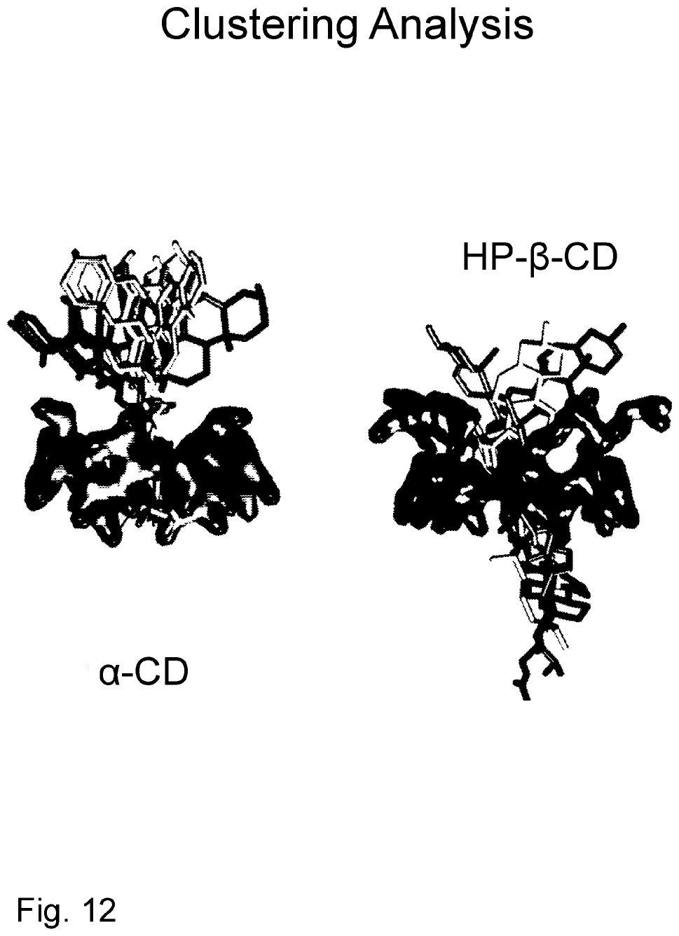

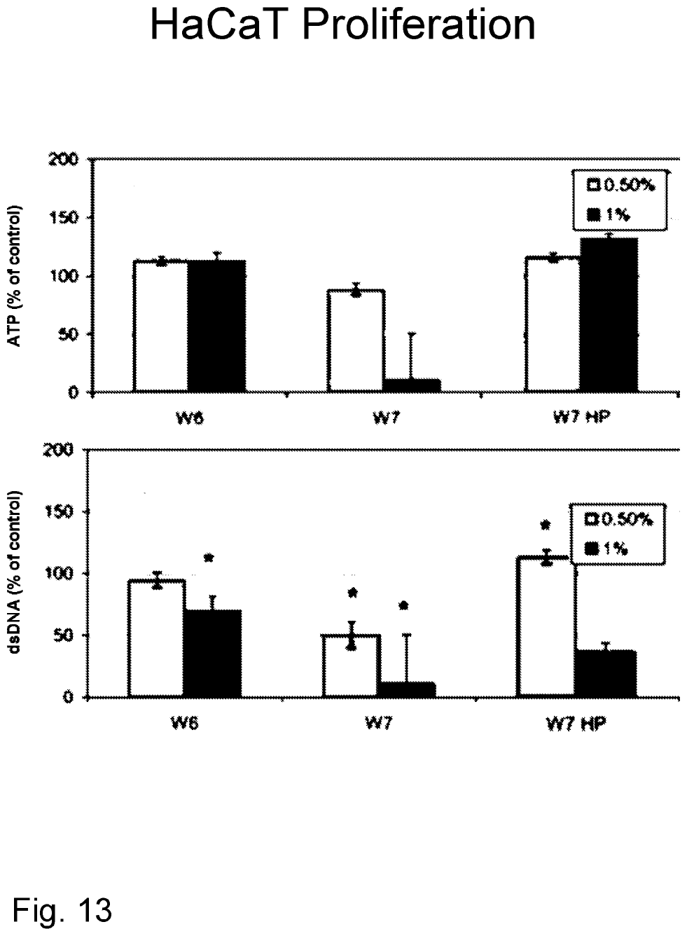

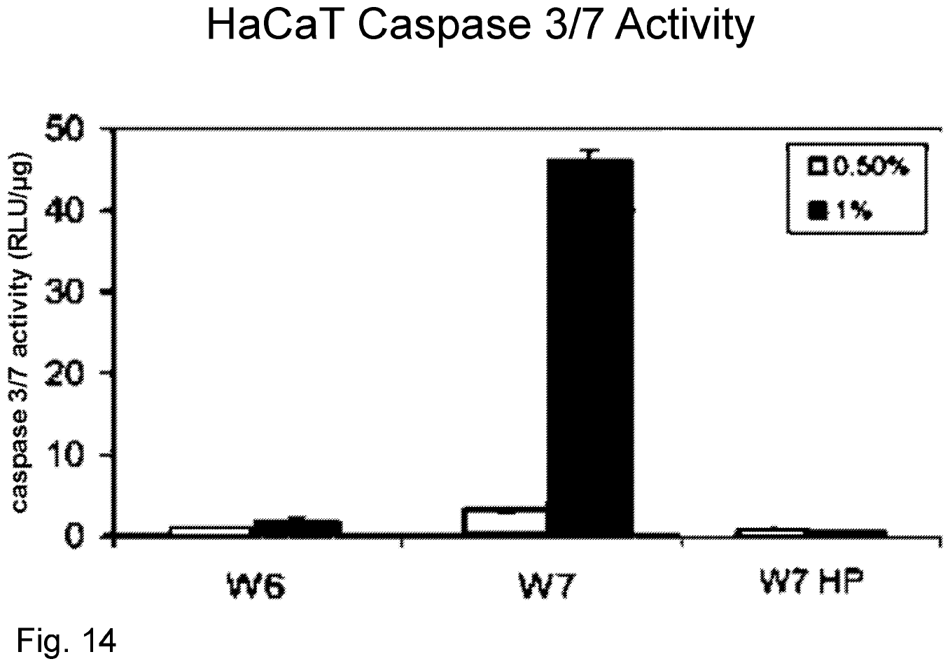

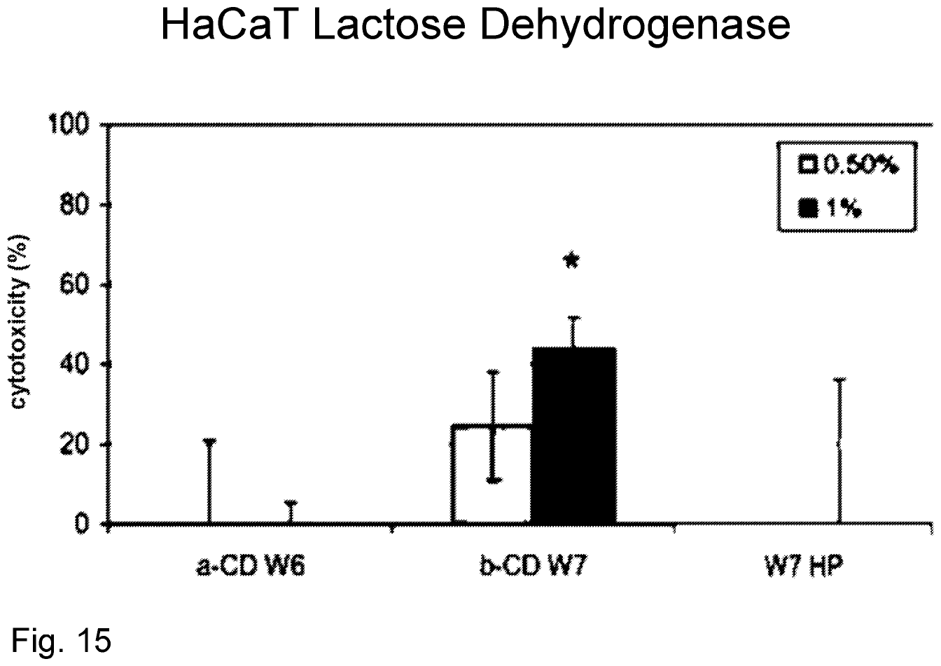

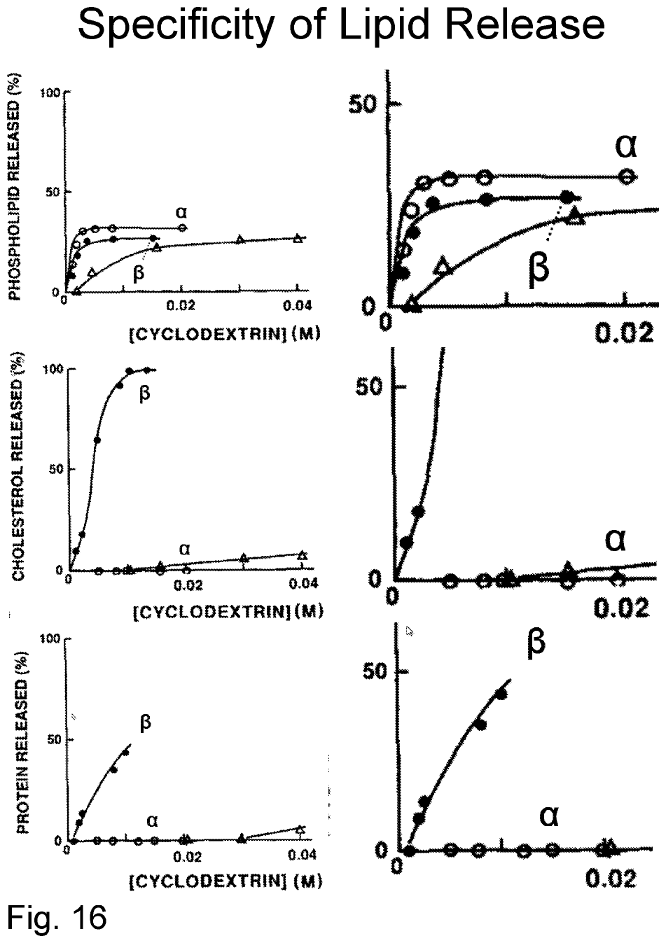

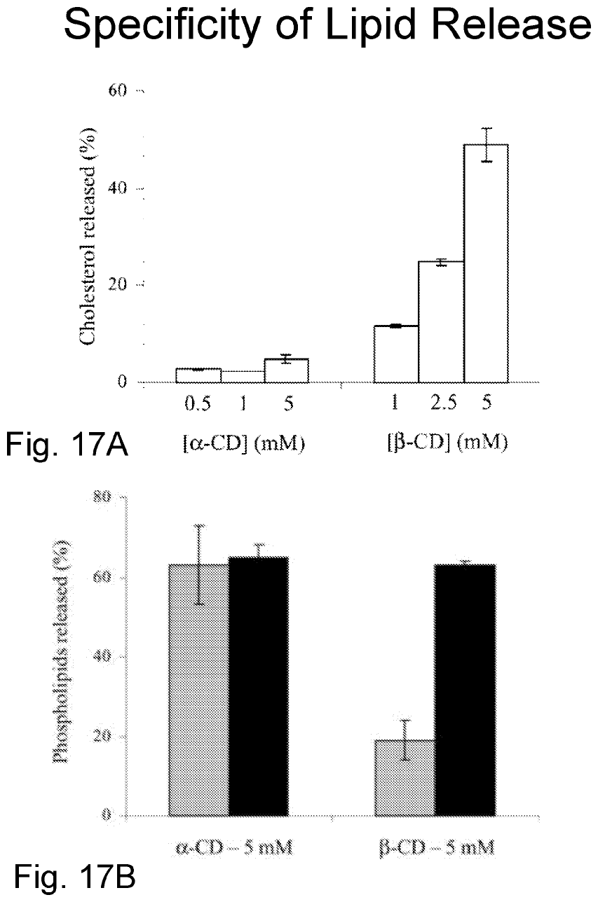

All cyclodextrins have the ability to scavenge phospholipids. β-cyclodextrins, however, have often been chosen over α-cyclodextrins because they can be used as expedients for more (larger) drugs. The smaller α-cyclodextrin (only six starch molecules, FIG. 11) has higher specificity for the smaller phospholipids (Ohtani et al. 1989; Vance et al. 2014), because cholesterol does not fit the smaller cavity of α-cyclodextrin (FIG. 12). The specifity of extraction of phospholipids, cholesterol, and proteins are shown in FIG. 16 (Ohtani et al. 1989). Among the phospholipids, α-cyclodextrin scavenges glycerolipids, such as the glycerolipids, more effectively than sphingolipids (FIG. 17). In particular, α-cyclodextrin is known to form complexes both around both the inositol head or the sn-2 chain of PI. (Fauvelle et al. 1997), Hence, α-cyclodextrin specifically downregulates the substrate for regulation of endocytosis via the PI cycle, thereby downregulating endocytosis with high specificity.

HPαCD retains the preference of α-cyclodextrin for phospholipids over cholesterol (Huang et al. 2013).

Ototoxicity of β-cyclodextrin is believed to be caused by β-cyclodextrin depriving prestin of the cholesterol it needs to function in outer hair cells (Takahashi et al. 2016; Kamar et al. 2012). As the cavity of α-cyclodextrin is too small for cholesterol, α-cyclodextrin and its derivatives, including, but not limited to, HP-α-cyclodextrin, are expected to avoid ototoxicity.

Long-term parenteral administration of HP-β-CD (200 mg/kg) was reported to decrease bone mineral density (BMD), which was associated with increased bone resorption (Kantner et al. 2012), while a CD-bisphosphonate conjugate, alendronate-β-CD (ALN-β-CD) was shown to be bone-anabolic (Liu et al. 2008).

Treatment of Cancer Patients with Alpha-Cyclodextrin or Derivatives or Salts Thereof

Several embodiments of the disclosure include the use of alpha-cyclodextrin and/or derivatives or pharmaceutically acceptable salt thereof.

Embodiments of the present disclosure are particularly useful to treat individuals who have cancer identified as having one or a plurality of cells with an abnormally high rate of endocytosis or exocytosis, or, in some embodiments, a dysfunctional PI cycle. In some embodiments, methods for treating an subject who has cancer comprise the steps of first identifying cancer as having a high rate of endocytosis or exocytosis, or, in some embodiments, a dysfunctional PI cycle, and then administering to such a subject a therapeutically effective amount of a cyclodextrin. In some preferred embodiments, the identification of cancer as having a high rate of endocytosis or exocytosis, or, in some embodiments, a dysfunctional PI cycle, is done by PET imaging, preferably using a fluorescent tag, antibody, or other agent that identifies mutations in the amino acid sequence of Table 5b or an amino acid sequence at least 70%, 80%, 90%, 95% 96%, 97%, 98%, or 99% homology to the amino acid sequences of Table 5b. In some embodiments, the cyclodextrin is effective to scavenge phospholipid in greater than 50% of cells in an in vitro migration assay at a concentration of less than 4 mM, 3 mM, 2 mM, or 1 mM. In some embodiments, the cyclodextrin or derivative or salt thereof is effective to inhibit migration of cells in greater than 50% of cells in an in vitro cell migration assay at a concentration of about 1 mM. In some embodiments, the cyclodextrin or derivative or salt thereof is effective to reduce cell migration in greater than 50% of cells in an in vitro cell migration assay at a concentration from about 0.5 to about 1.5 mM.

In some embodiments of the present invention, methods for treating an individual who has been identified as having cancer comprise administering to such an individual a therapeutically effective amount of the cyclodextrin or derivative or salt thereof which is known to be effective to inhibit cell migration in greater than 50% of cells in an in vitro migration assay at a concentration of less than 1 mM. In some preferred embodiments, the cyclodextrin or derivative or salt thereof is effective to inhibit cell migration in greater than 50% of cells in an in vitro cell migration assay at a concentration of less than about 1.5 mM. In some preferred embodiments, the cyclodextrin or derivative or salt thereof is effective to inhibit cell migration in greater than about 50%, 60%, 70%, 80%, or 90% of cells in an in vitro cell migration assay at a concentration of about 1 mM. In some preferred embodiments, prior to administration of the cyclodextrin or derivative or salt thereof, the cancer is confirmed as being a cancer comprising one or a plurality of cells characterized by an abnormally high rate of endocytosis or exocytosis, or, in some embodiments, a dysfunctional PI cycle. The preferred method of doing so is be PET imaging, polymerase chain reaction (PCR) of a sample such as a biopsy.

Methods are provided for inhibiting, even partially, metastasis of a cancer cell. The methods comprise delivering to the cancer cell an amount of cyclodextrin or derivative or salt thereof effective to inhibit cell migration of the cell. The cyclodextrin or derivative or salt thereof used is effective to slow migration of a cancer cell in greater than 50% of cells in an in vitro cell migration assay at a concentration of less than about 2 mM or about 1.5 mM. In some preferred embodiments, the cyclodextrin or derivative or salt thereof is effective to inhibit cell migration in greater than 50% of cells in an in vitro cell migration assay at a concentration of less than 1.5 mM. In some preferred embodiments, the cyclodextrin or derivative or salt thereof is effective to inhibit cell migration in greater than 50% of cells in an in vitro cell migration assay at a concentration of at or about less than 1.0 mM. In some embodiments, the treatment simultaneously reduces the ototoxicity of the treatment.

Embodiments of the present invention are particularly useful to treat patients who have cancer with cancer cells that have abnormally high rate of endocytosis or exocytosis, or, in some embodiments, a dysfunctional PI cycle. Such cancers include most cancers and generally exclude those cancers arising from tissues associated with lipid production such as liver cancer, and cancer involving fat cells. Cancer cells that have abnormally high rate of endocytosis or exocytosis, or, in some embodiments, a dysfunctional PI cycle, are generally limited to epithelial cell derived cancers. In some embodiments, cancer is from epithelial cells of the breast, colon, lung, or prostate. Thus, some methods of the invention relate to methods of treating a cancer patient who has cancer that have abnormally high rate of endocytosis or exocytosis, or, in some embodiments, a dysfunctional PI cycle, wherein such methods comprise the step of administering to such patient or subject a therapeutically effective amount of cyclodextrin. In preferred embodiments, the cyclodextrin or derivative or salt thereof is known to be effective to slow cell migration in greater than 50% of cells in an in vitro cell migration assay at a concentration of less than 2 mM. In some preferred embodiments, the cyclodextrin or derivative or salt thereof is effective to induce apoptosis in greater than about 50%, 60%, 70%, 80%, or 90% of cells in an in vitro cell migration at a concentration of less than 1.5 mM. Cancer cells that have abnormally high rate of endocytosis or exocytosis, or, in some embodiments, a dysfunctional PI cycle, generally have dysfunction of enzymes of Table 5b and/or metabolize and/or uptake high levels of phospholipids around their microenvironment. In some preferred embodiments, prior to administration of cyclodextrin or derivative or pharmaceutically acceptable salt thereof, the cancer is confirmed as being a cancer characterized by an abnormally high rate of endocytosis or exocytosis, or, in some embodiments, a dysfunctional PI cycle. The preferred method of doing so is be PET imaging, PCR or immunohistochemistry of a sample.

Methods are provided for preventing or inhibiting the rate of metastases of a cancer cell characterized by an abnormally high rate of endocytosis or exocytosis, or, in some embodiments, a dysfunctional PI cycle. The methods comprise delivering to the cancer cell an amount of a cyclodextrin or derivative or pharmaceutically acceptable salt thereof effective to reduce cell migration of the cell. The cyclodextrin or derivative or pharmaceutically acceptable salt thereof used is effective to inhibit or reduce the rate of cell migration in greater than about 50%, 60%, 70%, 80%, or 90% of cells in an in vitro cell migration assay at a concentration of less than 1.5 mM. In some preferred embodiments, the disclosed treatment herein is effective to inhibit or reduce the rate of cell migration in greater than about 50%, 60%, 70%, 80%, or 90% of cells in an in vitro cell migration assay at a concentration of less than 1.1 mM. In some preferred embodiments, the disclosed treatment herein is effective to inhibit or reduce the rate of cell migration in greater than about 50%, 60%, 70%, 80%, or 90% of cells in an in vitro cell migration assay at a concentration of less than about 1.0 mM.

In some embodiments, any of the methods disclosed herein are free of administration of a cyclodextrin that scavenges cholesterol upon administration to a subject.

The production of cyclodextrins is relatively simple and involves treatment of ordinary starch with a set of easily available enzymes, Commonly cyclodextrin glycosyltransferase (CGTase) is employed along with α-amylase. First starch is liquefied either by heat treatment or using α-amylase, then CGTase is added for the enzymatic conversion. CGTases can synthesize all forms of cyclodextrins, thus the product of the conversion results in a mixture of the three main types of cyclic molecules, in ratios that are strictly dependent on the enzyme used: each CGTase has its own characteristic α:β:γ synthesis ratio. Purification of the three types of cyclodextrins takes advantage of the different water solubility of the molecules: β-CD which is very poorly water-soluble (18.5 g/l or 16.3 mM) (at 25 C) can be easily retrieved through crystallization while the more soluble α- and γ-CDs (145 and 232 g/l respectively) are usually purified by means of expensive and time consuming chromatography techniques. As an alternative a “complexing agent” can be added during the enzymatic conversion step: such agents (usually organic solvents like toluene, acetone or ethanol) form a complex with the desired cyclodextrin which subsequently precipitates. The complex formation drives the conversion of starch towards the synthesis of the precipitated cyclodextrin, thus enriching its content in the final mixture of products. Wacker Chemie AG uses dedicated enzymes, that can produce alpha-, beta- or gamma-cyclodextrin specifically. This is very valuable especially for the food industry, as only alpha- and gamma-cyclodextrin can be consumed without a daily intake limit.

### B) Pharmaceutical Compositions and Routes of Administration

In some embodiments, the present invention provides compositions comprising any one or more of the active agents described herein, either alone or in combination, for example for use in treating a malignant or neurodegenerative disease or disorder. For example, in some embodiments, the present invention provides compositions comprising a cyclodextrin, or an analogue or derivative thereof, for example for use in treating breast or prostate cancer.

Pharmaceutical compositions provided by the present disclosure include compositions wherein the active ingredient (e.g. compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule (e.g. PIP, PIP2, PIP3), and/or reducing, eliminating, or slowing the progression of disease symptoms (e.g. symptoms of malignant disorders or a neurodegeneration such as symptoms of Parkinson's disease). Determination of a therapeutically effective amount of a compound of the disclosure is well within the capabilities of those skilled in the art, especially in light of the detailed disclosure herein.

The pharmaceutical composition may be formulated by one having ordinary skill in the art with compositions selected depending upon the chosen mode of administration. Suitable pharmaceutical carriers are described in the most recent edition of Remington's Pharmaceutical Sciences, A. Osol, a standard reference text in this field.

Administering the pharmaceutical composition can be effected or performed using any of the various methods known to those skilled in the art. Systemic formulations include those designed for administration by injection, e.g. subcutaneous, intravenous, intramuscular, intrathecal or intraperitoneal injection, as well as those designed for transdermal, transmucosal, oral or pulmonary administration.

Pharmaceutical compositions of the invention may be administered by a variety of routes including oral, buccal, sublingual, rectal, transdermal, subcutaneous, intravenous, intramuscular, intrathecal, intraocular, intraperitoneal and intranasal. Depending on whether intended route of delivery is oral or parenteral, the active agents can be formulated as compositions that are, for example, either injectable, topical or oral compositions. Liquid forms of compositions may include a suitable aqueous or nonaqueous vehicle with buffers, suspending and dispensing agents, colorants, flavors and other suitable ingredients known in the art. Solid forms of compositions may include, for example, binders, excipients, lubricants, coloring agents, flavoring agents and other suitable ingredients known in the art. The active agents and pharmaceutical compositions of the invention may also be administered in sustained release forms or from sustained release drug delivery systems known in the art.

In some embodiments the compositions of the present invention are pharmaceutical compositions comprising one or more active agents, as described herein, together with one or more conventionally employed components suitable for use in pharmaceutical delivery such as pharmaceutically acceptable carriers, stabilizers, diluents, dispersing agents, suspending agents, thickening agents, excipients, and the like may be placed into the form of pharmaceutical formulations. In some embodiments, the disclosure relates to pharmaceutical compositions comprising a therapeutically effective amount of α-cyclodextrin and at least one or a plurality of pharmaceutically acceptable carriers. Nonlimiting examples of such formulations include solutions, creams, gels, gel emulsions, jellies, pastes, lotions, salves, sprays, ointments, powders, solid admixtures, aerosols, emulsions (e.g., water in oil or oil in water), gel aqueous solutions, aqueous solutions, suspensions, liniments, tinctures, and patches suitable for topical administration. The pharmaceutical compositions and formulations of the invention may, where appropriate, be conveniently presented in discrete unit dosage forms and may be prepared by any of the methods well known in the art of pharmacy. Such methods include the step of bringing into association an active agent with liquid carriers, solid matrices, semi-solid carriers, finely divided solid carriers or combinations thereof, and then, if necessary, shaping the product into the desired delivery system. Unit dosage forms of a pharmaceutical composition or formulation preferably contain a predetermined quantity of active agent and other ingredients calculated to produce a desired therapeutic effect, such as an effective amount of a therapeutically effective amount. Typical unit dosage forms include, for example, prefilled, premeasured ampules or syringes of liquid compositions, or pills, tablets, capsules or the like for solid compositions.

For injection, the compounds of the invention may be formulated in aqueous solutions, preferably in physiologically compatible buffers such as Hanks's solution, Ringer's solution, or physiological saline buffer. The solution may contain formulatory agents such as suspending, stabilizing and/or dispersing agents. Injectables are sterile and pyrogen free. Alternatively, the compounds may be in powder form for constitution with a suitable vehicle, e.g., sterile pyrogen-free water, before use. For transmucosal administration, penetrants appropriate to the barrier to be permeated are used in the formulation. Such penetrants are generally known in the art.

For parenteral administration, the cyclodextrin or derivative thereof can be, for example, formulated as a solution, suspension, emulsion or lyophilized powder in association with a pharmaceutically acceptable parenteral vehicle or pharmaceutically acceptable carrier. Examples of such vehicles or carriers are water, saline, Ringer's solution, dextrose solution, 5% human serum albumin, Ringer's dextrose, dextrose and sodium chloride, lactated Ringer's and fixed oils, polyethylene glycol, polyvinyl pyrrolidone, lecithin, arachis oil or sesame oil. Liposomes and nonaqueous vehicles such as fixed oils may also be used. The vehicle or lyophilized powder may contain additives that maintain isotonicity (e.g., sodium chloride, mannitol) and chemical stability (e.g., buffers and preservatives). The formulation is sterilized by commonly used techniques. Parenteral dosage forms may be prepared using water or another sterile carrier. For example, a parenteral composition suitable for administration by injection is prepared by dissolving 1.5% by weight of active ingredient in 0.9% sodium chloride solution. Alternatively, the solution can be lyophilised and then reconstituted with a suitable solvent just prior to administration.

Pharmaceutically acceptable carriers are well known to those skilled in the art and include, but are not limited to, from about 0.01 to about 0.1 M or about 0.05 M phosphate buffer or about 0.8% saline. Intravenous carriers include fluid and nutrient replenishers, electrolyte replenishers such as those based on Ringer's dextrose, and the like. Additionally, such pharmaceutically acceptable carriers can be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, ethanol, alcoholic/aqueous solutions, glycerol, emulsions or suspensions, including saline and buffered media.

The pharmaceutical compositions can be prepared using conventional pharmaceutical excipients and compounding techniques. Oral dosage forms may be elixers, syrups, tablets, pills, dragees, capsules, liquids, gels, syrups, slurries, suspensions and the like, for oral ingestion by a patient to be treated. The typical solid carrier may be an inert substance such as lactose, starch, glucose, cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; binding agents, magnesium sterate, dicalcium phosphate, mannitol and the like. A composition in the form of a capsule can be prepared using routine encapsulation procedures. For example, pellets containing the active ingredient can be prepared using standard carrier and then filled into a hard gelatin capsule; alternatively, a dispersion or suspension can be prepared using any suitable pharmaceutical carrier(s), for example, aqueous gums, celluloses, silicates or oils and the dispersion or suspension then filled into a soft gelatin capsule. Typical liquid oral excipients include ethanol, glycerol, glycerine, non-aqueous solvent, for example, polyethylene glycol, oils, or water with a suspending agent, preservative, flavoring or coloring agent and the like. All excipients may be mixed as needed with disintegrants, diluents, lubricants, and the like using conventional techniques known to those skilled in the art of preparing dosage forms. If desired, disintegrating agents may be added, such as the cross-linked polyvinylpyrrolidone, agar, or alginic acid or a salt thereof such as sodium alginate. If desired, solid dosage forms may be sugar-coated or enteric-coated using standard techniques. For oral liquid preparations such as, for example, suspensions, elixirs and solutions, suitable carriers, excipients or diluents include water, glycols, oils, alcohols, etc. Additionally, flavoring agents, preservatives, coloring agents and the like may be added.

For buccal administration, the compositions of the disclosure may take the form of tablets, lozenges, and the like formulated in conventional manner. The compounds may also be formulated in rectal or vaginal compositions such as suppositories or enemas. A typical suppository formulation comprises a binding and/or lubricating agent such as polymeric glycols, glycerides, gelatins or cocoa butter or other low melting vegetable or synthetic waxes or fats. For administration by inhalation, the compounds for use according to the present invention are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The formulations may also be a depot preparation which can be administered by implantation (for example subcutaneously or intramuscularly) or by intramuscular injection. In such embodiments, the compounds may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems may be employed. Liposomes and emulsions are well known examples of delivery vehicles that may be used. Certain organic solvents such as dimethylsulfoxide also may be employed, although usually at the cost of greater toxicity. Additionally, the compounds may be delivered using a sustained-release system, such as semipermeable matrices of solid polymers containing the therapeutic agent. Various of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The compounds used in the invention may also be formulated for parenteral administration by bolus injection or continuous infusion and may be presented in unit dose form, for instance as ampoules, vials, small volume infusions or pre-filled syringes, or in multi-dose containers with an added preservative.

Preservatives and other additives can also be present, such as, for example, antimicrobials, antioxidants, chelating agents, inert gases and the like. All carriers can be mixed as needed with disintegrants, diluents, granulating agents, lubricants, binders and the like using conventional techniques known in the art.

### C) Dosages

The dose of an active agent of the invention may be calculated based on studies in humans or other mammals carried out to determine efficacy and/or effective amounts of the active agent (see section E, Clinical Outcomes, below). The dose amount and frequency or timing of administration may be determined by methods known in the art and may depend on factors such as pharmaceutical form of the active agent and route of administration, and patient characteristics including age, body weight or the presence of any medical conditions affecting drug metabolism.

In one embodiment, a dose may be administered as a single dose. In another embodiment, a dose may be administered as multiple doses over a period of time, for example, at specified intervals, such as, daily, bi-weekly, weekly, monthly, and the like. In another embodiment, the dose will be about 700 mg/kg/d. In another embodiment, the doses comprise multiple ascending doses (increased over time) until early signs of renal or cytotoxicity are observed, in which case the dose level will be decreased to the previous, well-tolerated level.

In one embodiment of the invention, the dose of active agent is at least about 10 mg, at least about 20 mg, at least about 30 mg, at least about 40 mg, at least about 50 mg, at least about 75 mg, at least about 100 mg, at least about 125 mg, at least about 150 mg, at least about 175 mg, at least about 200 mg, at least about 225 mg, at least about 250 mg, at least about 275 mg, at least about 300 mg, at least about 325 mg, at least about 350 mg, at least about 375 mg, at least about 400 mg, at least about 425 mg, at least about 450 mg, at least about 475 mg, at least about 500 mg, at least about 550 mg, at least about 600 mg, at least about 650 mg, at least about 700 mg, at least about 750 mg, at least about 800 mg, at least about 850 mg, at least about 900 mg, at least about 950 mg, at least about 1000 mg, at least about 1200 mg, at least about 1500 mg, at least about 2000 mg, at least about 2500 mg, at least about 3000 mg, at least about 4000 mg, at least about 5000 mg, at least about 7500 mg, at least about 10,000 mg, at least about 15,000 mg, at least about 20,000 mg, or at least about 25,000 mg. In some such embodiments the above dosages are mg/day or mg/kg/day. In another embodiment, the dose of active agent is in the range of 1 to 10000 mg, 1 to 7500 mg, 1 to 5000 mg, 1 to 2500 mg, 1 to 1000 mg, 1 to 500 mg, 1 to 250 mg, 250 to 10000 mg, 250 to 5000 mg, 250 to 1000 mg, 250 to 500 mg, 500 to 10000 mg, 500 to 5000 mg, 500 to 1000 mg. In some embodiments the above dosages are mg/day or mg/kg/day.

In one embodiment, a single dose may be administered. In another embodiment, multiple doses may be administered over a period of time, for example, at specified intervals, such as, four times per day, twice per day, once a day, weekly, monthly, 4 times over 14 days, 2 times over 21 days, twice per month, 4 times over 21 days, 4 times per month, or 5, 6, 7, 8, 9, 10, 11, 12 or more times per month, per 21 days, per 14 days, or per week, and the like.

In one embodiment, the doses administered intravenously or intrathecally may be about 5600 mg/kg/wk or about 400 mg/wk (Table 3).

TABLE 3
Hempel twins dose adjustment

| Date | Description |
|---|---|
| 2004 January | Birth |
| 2008 Aug. 14 | confirmed diagnosis of NPC |
| 2009 Feb. 22 | HP-β -CD treatment plan |
| | Initial infusion: 4 d continuous IV of 80 mg/kg/d at a rate of 20 ml/hr |
| | Next: Weekly 8 h infusions starting at 160 mg/kg/d + add'l weekly infusions |
| | Next: 320 mg/kg/d |
| | Next 400 mg/kg/d |
| 2009 Apr. 13 | Approval of i.v. infusion (INDs 104,114 and 104,116) |
| 2009 Jul. 7 | Protocol extension |
| | 400 mg/kg/d administered as a weekly eight hour infusion |
| 2009 Jul. 16 | Increased dosing frequency (twice/week) and rate of dose titration (100 |
| | mg/kg/infusion) |
| | Week 1: 500 mg/kg/d; 8 hrs × 1 + 3-4 days 600 mg/kg/d; 8 hrs × 1 |
| | Week 2: 700 mg/kg/d; 8 hrs × 1 + 3-4 days 800 mg/kg/d; 8 hrs × 1 |
| | Week 3: 900 mg/kg/d; 8 hrs × 1 + 3-4 days 1000 mg/kg/d; 8 hrs × 1 |
| | Initial infusion: 500 mg/kg/d over 8 hrs at a rate of 20 ml/hr |
| 2009 Oct. 8 | Pulmonary clinic visit |
| | 2800 mg/kg twice weekly over 8 hr (800 mg/kg/d) |
| 2010 Mar. 7 | “2-Hydroxypropyl-β-Cyclodextrin Raises Hearing Threshold in . . . Cats.” |
| | (Ward et al. 2010) |
| 2010 Apr. 15 | Hearing unaffected despite receiving steady state IV doses of |
| | 2.5 g/kg bi-weekly for >1 yr (5 g/kg/wk ≈700 mg/kg/d) |
| 2010 May 17 | Orphan-drug designation granted |
| 2010 Aug. 13 | Request for intrathecal delivery filed |
| | 200 mg HP-β-CD intrathecal biweekly (≈60 mg/kg/d) |
| | (Maarup et al. 2015) |

From Hastings C (2009 Feb. 22) Addi and Cassi Hydroxy-Propyl-Beta-Cyclodextrin Plan.
Compassionate Use Clinical Study. Treatment Plan Version #2, http://addiandcassi.com/wordpress/wp-content/uploads/2009/09/FDA-Submission-for-Addi-and-Cassi-Cyclodextrin-Treatment-Plan.pdf
NPC: http://www.nnpdf.org/cyclodextrin.html

“Phospholipid exchange by HP-α-cyclodextrin and HP-β-cyclodextrin seemed to be similar, despite the difference in their ring sizes” (Huang et al. 2013).

### D) Subjects

The methods and compositions provided by the invention may be used to treat a malignant or neurodegenerative disease or disorder, such as breast or prostate cancer, in any subject in need of such treatment. In one embodiment, the subject is a human. It should be noted that, while in some embodiments the subjects to be treated are post-menopausal women, in other embodiments the methods of treatment described herein are not intended to be limited to such subjects. Rather, in some embodiments the subjects can be of any age, ranging from newborns to older adults. In some embodiments it may be desirable to treat young subjects, for example young infants, particularly where family history or genetic testing indicates that the subject is at risk for developing a malignant or neurodegenerative disease or disorder. Similarly, in some embodiments it may be desirable to treat much older subjects, particularly where such subjects begin to exhibit indicators or symptoms of a malignant or neurodegenerative disease or disorder.

The methods and compositions of the invention may be employed as prophylactic treatments or therapeutic treatments. For prophylactic treatments, the methods and compositions provided herein can be used preventatively in subjects that do not yet exhibit any clear or detectable clinical indicators or symptoms of a malignant or neurodegenerative disease or disorder but that are believed to be at risk of developing a malignant or neurodegenerative disease or disorder, such as breast or prostate cancer. A subject receiving prophylactic treatment for breast or prostate cancer, for example, may not exhibit any clinical indicators or symptoms of breast or prostate cancer. In the case of therapeutic treatments, the methods and compositions provided herein can be used in subjects that already exhibit one or more clinical indicators or symptoms of the disease or disorder, such as breast or prostate cancer. A subject receiving therapeutic treatment for breast or prostate cancer, for example, may have been clinically diagnosed with breast or prostate cancer or may otherwise exhibit one or more clinical indicators or symptoms of breast or prostate cancer.

In one embodiment of the invention, a subject may have been identified as being at risk of developing breast or prostate cancer derived from breast or prostate tissue. In one embodiment, the subject has a family history of breast or prostate cancer. In one embodiment, the subject has one or more genetic risk factors associated with breast or prostate cancer, for example, a genetic mutation in a gene associated with PIP cycling.

### E) Clinical Outcomes

In some embodiments the methods of treatment provided herein (which comprise, for example, administering to a subject an effective amount of a composition according to the present invention) result in, or are aimed at achieving, a detectable improvement in one or more clinical indicators or symptoms of cancer, including, but not limited to, changes growth, migration, or invasion. In some embodiment of the present invention a symptom or indicator of improvement is selected from the group comprising survival, disease-free survival, distant metastasis-free survival, results of a blood test (including, but not limited to circulating tumor DNA and prostate-specific antigen), an x-ray evaluation, the result of a physical examination (including, but not limited to a palpable tumor), or a tissue biopsy for histological evaluation.

To determine the highest tolerated dose in an individual complete blood count and serum chemistry will be collected and analyzed. The serum chemistries may include, but will not be limited to evaluation of electrolytes, bicarbonate, glucose, BUN, creatinine, magnesium, phosphate, hepatic enzymes (AST and ALT), total protein, albumin, bilirubin, and alkaline phosphatase. In addition, a complete lipid panel may be obtained and shape of erythrocytes may be evaluated microscopically. Bone density may be measured to identify early signs of osteoporosis.

The compositions and methods described herein are illustrative only and are not intended to be limiting. Those of skill in the art will appreciate that various combinations or modifications of the specific compositions and methods described above can be made, and all such combinations and modifications of the compositions and methods described herein may be used in carrying out the present invention. Furthermore, certain embodiments of the present invention are further described in the following non-limiting Examples, and also in the following Claims.

### Related Patents

In the context of AD, decreasing phosphatic acid (PA) has been declared desirable before (U.S. Pat. No. 8,288,378): “Agents which decrease PA levels include, but are not limited to, an inhibitor of diacylglycerol kinase, an inhibitor of phospholipase D1, and/or an inhibitor of phospholipase D2. [ . . . ] Non-limiting examples of [such agents] include, but are not limited to, siRNA directed toward phospholipase D1 or D2 or diacylgerycerol [sic].” No such agents were given in (U.S. Pat. No. 8,288,378), which has not been cited.

No patent from “alpha-cyclodextrin” is related.

### EXAMPLES

### Example 1: The PI-Cycle is a Drug Target Against Metastases in Breast Cancer

The methods used to obtain the results presented in Example 1 are further described in Wittkowski et al. 2014, which is hereby incorporated by reference in its entirety.

Almost a decade after the completion of the Human Genome Project, the scientific and medical advances hoped for from genome-wide association studies (GWAS) have not yet been realized. Enlarging the sample size to tens of thousands of subjects greatly increases the duration and cost of data collection and, in a nonrandomized study, may somewhat paradoxically increase the risk of false positives. This Example describes combining a novel computational biostatistics approach with decision strategies fine-tuned to the exploratory nature of GWAS. With these methodological advances, disease-relevant functional gene clusters can now be suggested from studies of a few hundred narrowly defined cases only.

Although a history of familial breast cancer being a known risk factor of either breast or prostate cancer attests to a high degree of heritability, the genetic risk factors for breast or prostate cancer in the general population are still poorly understood. As described herein, data from three independent populations (available from NIH's dbGaP) were analyzed using u-statistics for genetically structured wide-locus data to explore epistasis. To account for systematic, but disease-unrelated differences in (non-randomized) genome-wide association studies (GWAS) and for conducting multiple tests in overlapping genetic regions, a novel study-specific criterion for ‘genome-wide significance’ was applied (Wittkowski et al. 2014). Enrichment of the results in all three studies with genes associated with different stages of endocytosis confirms the hypothesis that control of endocytoses through PI cycling is involved in metastases as the process turning breast or prostate cancer into a deadly disease.

The approach used here had been validated in childhood absence epilepsy (Wittkowski et al. 2013) and then generated a novel testable hypothesis about preventing mutism in autism (Wittkowski et al. 2014). With the additional evidence in these studies on breast cancer and the genetic data from many more studies are already publicly available, the described computational biostatistics approach will advance personalized medicine and comparative effectiveness research. The genetic data collected over the last decade, could finally yield profound insights into the mechanistic bases of many common diseases and subgroup analyses of phase II and phase III trials can now suggest risk factors for adverse events and novel directions for drug development.

Breast or prostate cancer are among the cancers with the highest mortality. Still, the genetic risk factors for the more common disease forms are still poorly understood.

### Subjects

The study was approved as appropriate. No human participants were involved in the research. The samples were genotyped on Human1Mv1_C and Human1M-Duov3_B Illumina chips.

The results described herein are based on three studies of breast cancer in the US and Europe. These studies included data from:
(1) the Cancer Genetic Markers of Susceptibility (CGEM) breast cancer genome-wide association study (http://www.ncbi.nlm.nih.gov/proj ects/gap/cgi-bin/study.cgi?study_id=phs000147.v3.p1), which included 1145 cases/1142 controls (Hunter et al. 2007)
   and from two substudies of the nested case-control and one case-control study of estrogen receptor negative breast cancer within the Breast and Prostate Cancer Cohort Consortium (http://www.ncbi.nlm.nih.gov/proj ects/gap/cgi-bin/study.cgi?study_id=phs000812.v1.p1), both included in (Garcia-Closas et al. 2013):
(2) the European Prospective Investigation into Cancer (EPIC) of 511 estrogen-receptor negative cases and 500 controls, and
(3) the Polish Breast Cancer Case-Control Study (PBCS) of 543 estrogen-receptor negative cases (229 triple-negative) and 511 controls.

### Methods

ssGWAS: After eliminating non-informative or low-quality SNPs, a traditional ssGWAS was performed, using u-statistics for univariate data (also known as the Mann-Whitney test (Mann et al. 1947) which is equivalent to the Wilcoxon rank-sum test (Wilcoxon 1954). By construction, the results of this analysis are very close to those obtained with the traditional Cochran-Armitage trend test (Armitage 1955).

Annotation: The annotation files available have proven to be inadequate to appropriately distinguish between genes (or splice variants of genes) that are too far away or close enough to be likely related to a SNP or region being implicated. On the other hand, diplotypes may span LD blocks outside of genes or their regulatory regions, in which case it is unlikely that the functional implication of the variation can be identified. Also, there may not be sufficient information avail-able to determine the function of a gene, in which case the funding would not be useful for identifying collections of functionally related genes. Hence, the results returned from the grid/cloud infrastructure, still need to be manually reviewed to resolve ambiguities regarding the annotation.

Wide-locus approach. To overcome several of the shortcomings seen in previous applications of single-SNP GWAS (ssGWAS) applied to common diseases, several strategies were combined at different stages of the analysis process. Wide-loci of up-to six neighboring SNPs were aimed at as a primary outcome and the same non-parametric GWAS approach was applied based on u-statistics for structured multivariate data (Hoeffding 1948) with genotypic structures (μGWAS) as in the previous childhood absence epilepsy (Wittkowski et al. 2013) and autism (Wittkowski et al. 2014) studies. To avoid spurious findings, loci outside of linkage-disequilibrium (LD) blocks containing genes with known function or adjacent to their 5′-end were excluded. Loci highly influenced by a single SNP only were also excluded, unless this SNP was implicated in more than one of the studiers or had been implicated in previous studies.

Information Content: In contrast to traditional regression methods, muGWAS provides an intrinsic measure of “information content”, which can be used to highlight regions with high significance, but low information content as likely artifacts. In the Manhattan plot, below, highly significant results with low information content are highlighted in red and excluded (crossed out in white), unless there is other supportive evidence, such as a nearby SNP that had previously been reported as associated with breast cancer or another cancer. Some regions with low information content are dominated by a single SNP or involve diplotypes spanning LD blocks without being within a gene or its regulatory region. Diplotypes may also be excluded if moving the window by one SNPs results in a large (more than 100 fold) change in significance. Of note, these manual intervention cannot cause false positive results and current research aims at formalizing more of these rules to facilitate interpretation and avoid false negative results.

MAF-significance correlation. With any finite sample size, the significance of a u- or rank test is limited. Hence, more significant results can only be obtained for SNPs with sufficiently high MAF. ssGWAS simulations were performed with 2,500,000 permuted phenotypes, comparing two groups of equal size for various MAFs. The 1-10⁻⁵ quantile of the permutation distribution drops from the expected s=−log 10 p=5.26 cut-off, which is routinely met for MAF>0.33, to 4.9 (n=1000 subjects), 4.7 (n=500), and 4.5 (n=300) for a MAF of 0.05. For the 7.5 level, the bias is projected to be even larger. Due to this MAF-significance correlation, the expected diagonal in a ssGWAS QQ plot under the null hypothesis that “no SNP is associated with the trait” (Pearson et al. 2008) turns into an expected curve dropping below the diagonal towards the end (Wittkowski et al. 2014).

Estimating the expected s-value (−log₁₀(p)) distribution from >10⁸ permutations to obtain stable estimates of the 1-10^{-7.5} quantile is neither practical, nor sufficient to avoid a biased selection of SNPs for limited tests. Due to the MAF-significance correlation, any SNP ‘significant’ when comparing observed phenotypes, is also more likely to be ‘significant’ with random phenotype permutations (Wittkowski et al. 2014).

Non-randomization bias. The reason for this curvature often not being recognized is that GWAS subjects are deterministically categorized based on their outcome (e.g., non-verbal vs. verbal), rather than randomly assigned to interventions (as in clinical trials). Any deterministically categorized populations, however, are expected to differ systematically in aspects related to neither the condition of interest nor common ancestry factors (which could potentially be accounted for through stratification). When the downward trend from using a limited test and the upward bias from deterministic selection are similar, the s-values may still appear to follow the diagonal, except for loci suggesting ‘true association’ (Pearson et al. 2008).

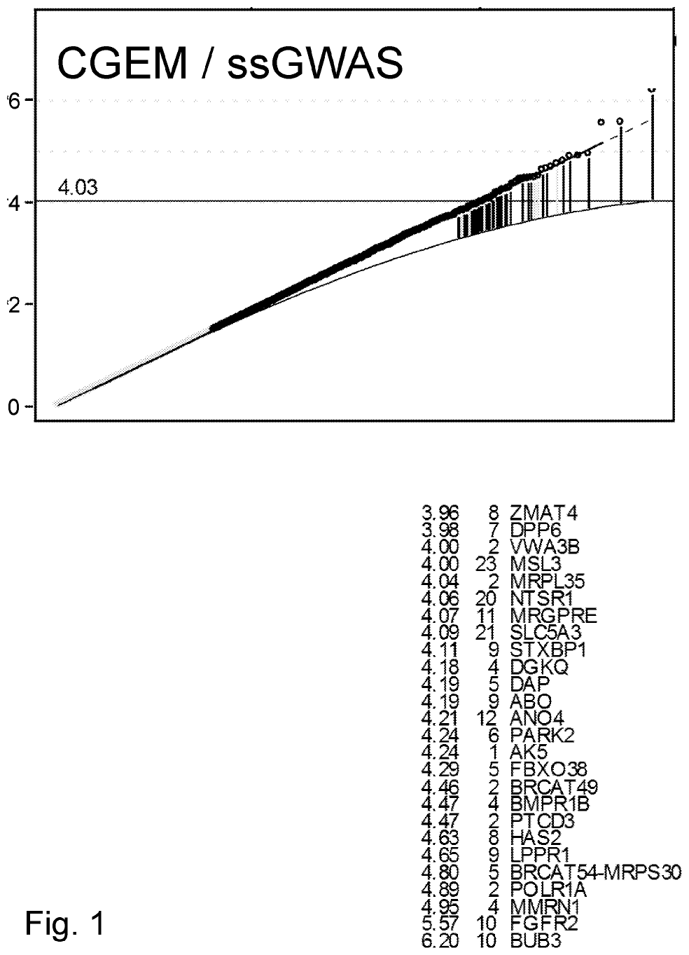

Multiplicity adjustments for diplotype length. For multivariate tests of overlapping diplotypes, the estimated quantile-rank (QR) curve needs to be elevated above the diagonal throughout to account for multiple tests conducted around the same SNP. Because most of these tests are highly dependent, the elevation of the estimated QR curve compared to the estimated QQ curve (FIG. 1-FIG. 6) is limited, but the distance is likely to vary across diseases and populations. (Wittkowski et al. 2014).

Projected QR curves. The diagonal of the traditional QQ-plot does not depend on any data, including the most ‘significant’ data. The s-values are expected to fit the diagonal for the most part (except for the most significant results) (Pearson et al. 2008), as the vast majority of SNPs are expected not to be associated with the disease. In direct analogy, the QR curve for a multivariate test should be ‘smooth’, with upward deviations indicating ‘true association’, which could be disease-related or not. Based on the above rationale and the simulation results mentioned above, the highest point of the projected QR curve (apex) for each chromosome can be estimated from a smooth projection of the s-values after truncating as many of the highest values as needed for the projection to have a monotone increase and, conservatively for a limited test, a non-positive second derivative. Fitting against the data also reduces the effect of population stratification (Pearson et al. 2008). (For computational convenience, locally weighted polynomial regression (Cleveland et al. 1988) was selected, as implemented in S+(TIBCO Software Inc.) as ‘loess. smooth( . . . degree=2, family=“gaussian”)).

Estimated whole genome QR apex. While chromosomes may differ with respect to their content of related and unrelated risk factors (see, e.g., the HLA region in autoimmune diseases), random errors are expected to have the same distribution across all chromosomes. Hence, the expected WG apex can be estimated as the (winsorized) median projected apex among chromosomes with the smallest deviation of s-values from the projection. (Here, ten chromosomes were selected based on the maximum norm, and the median for robustness, but the strategy to determine the optimal number, including the criteria for ‘optimality’, remains to be determined (Wittkowski et al. 2014).

Estimated QR curves. The estimated curve for each chromosome is then calculated as the loess projection (Cleveland et al. 1988) of this chromosome's s-values with as many of the highest values replaced with the estimated WG apex until the curve's apex is at or below that level. Applied to the WG projection (QR plots, bottom right), this procedure yields the estimated WG curve. Simulation results demonstrate the low variance of the estimates from phenotype permutations and the similarity of their median apex with the winsorized median apex estimated from the observed s-values (Wittkowski et al. 2014).

Study-specific genome-wide significance. For studies aiming to confirm individual SNPs as associated with a phenotype, the ‘confirmatory’ paradigm (Tukey 1980) requires adjustment for multiplicity. When applied to GWAS, these adjustments are typically based on a ‘customary’ fixed 0.05 level, irrespective of study size or relative risk of type I over type II errors (see Fisher 1956, p. 358, and Gigerenzer 2004, for a discussion), and the assumption of 1,000,000 independent SNPs, irrespective of chip density (Pearson et al. 2008). Moving from individual SNPs to overlapping diplotypes increases the dependency of any formal multiplicity adjustment on assumptions with questionable biological validity (Wittkowski et al. 2014).

As in most GWAS, however, the studies described in this Example do not aim to confirm hypotheses regarding specific SNPs. Instead, the studies described here aim at picking likely candidates from >40,000 (pseudo-) genes, whose relative importance and epistatic interactions are unknown. Since graphical procedures are particularly useful for such ‘exploratory’ studies (Tukey 1977), QR plots were chosen to guide with interpretation. Exact cut-offs for deviation of s-values from the estimated curve are unknown. When “the knowledge [is] at best approximate[,] an approximate answer to the right question, which is of—ten vague, [is far better] than an exact answer to the wrong question, which can always be made precise” (Tukey 1962). Hence, a heuristic approach is presented that relies on fewer unrealistic assumptions than typical attempts to quantify a particular error rate (Wittkowski et al. 2014).

The expected WG curve needs to be estimated, the s-values have a complex dependency structure, and the appropriate level of significance (α) for the given sample size is unknown. Hence, a heuristic decision rule is proposed based on weak assumptions only. In the long run one would expect most s-values above the apex to be significant at any α>0 (consistency) and regions with the strongest association to have the highest odds at being included (unbiasedness). For a particular α, one could lower the cut-off, but to account for variance in estimating the apex, one would need to raise it. As a compromise, the estimated WG apex is proposed as a cut-off for study-specific GWS (Wittkowski et al. 2014).

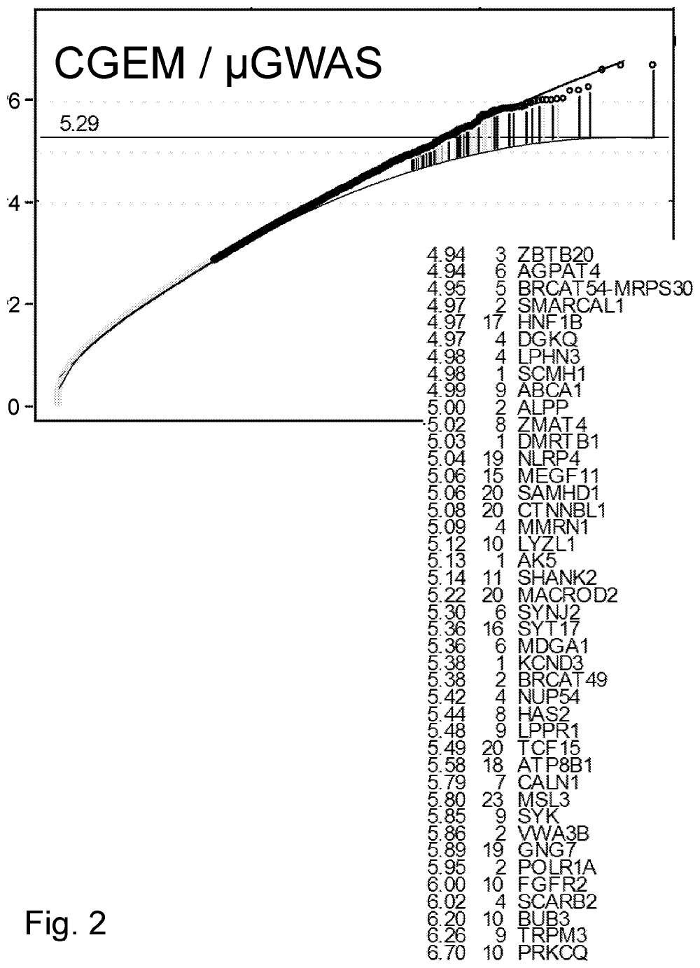

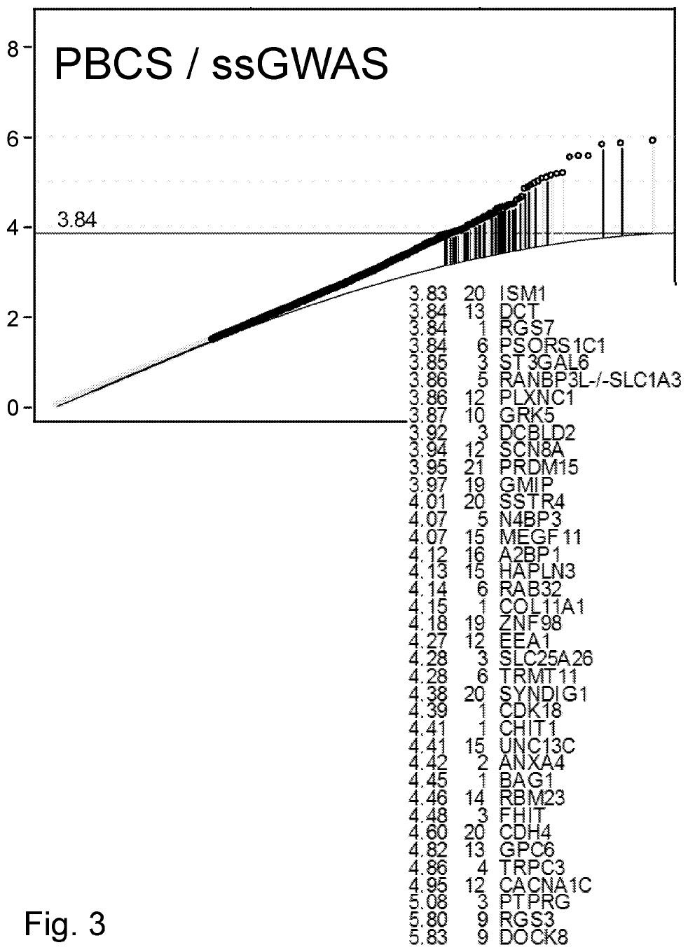

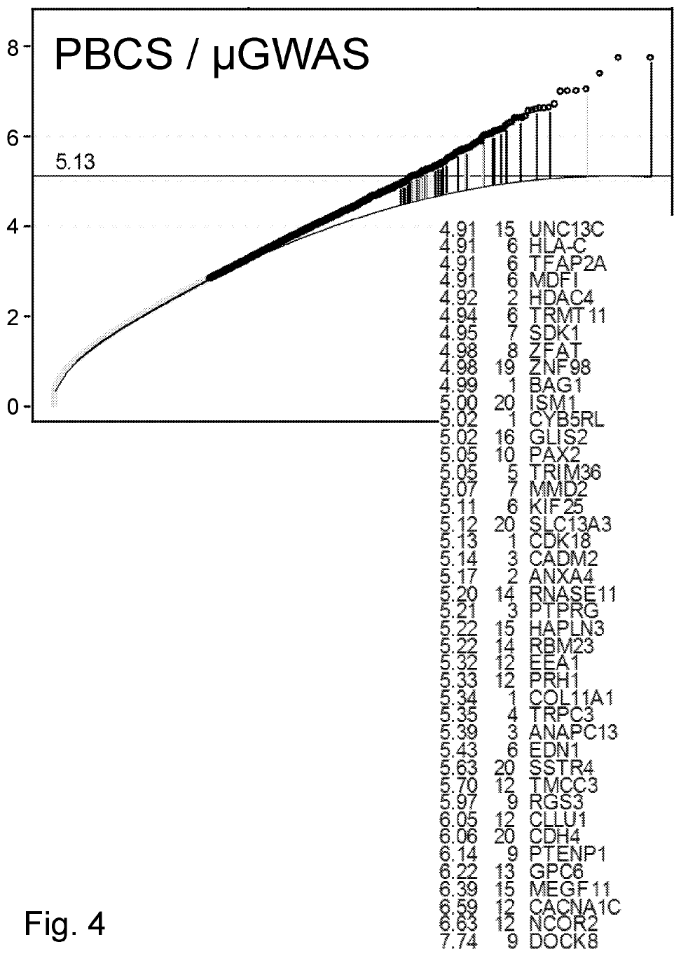

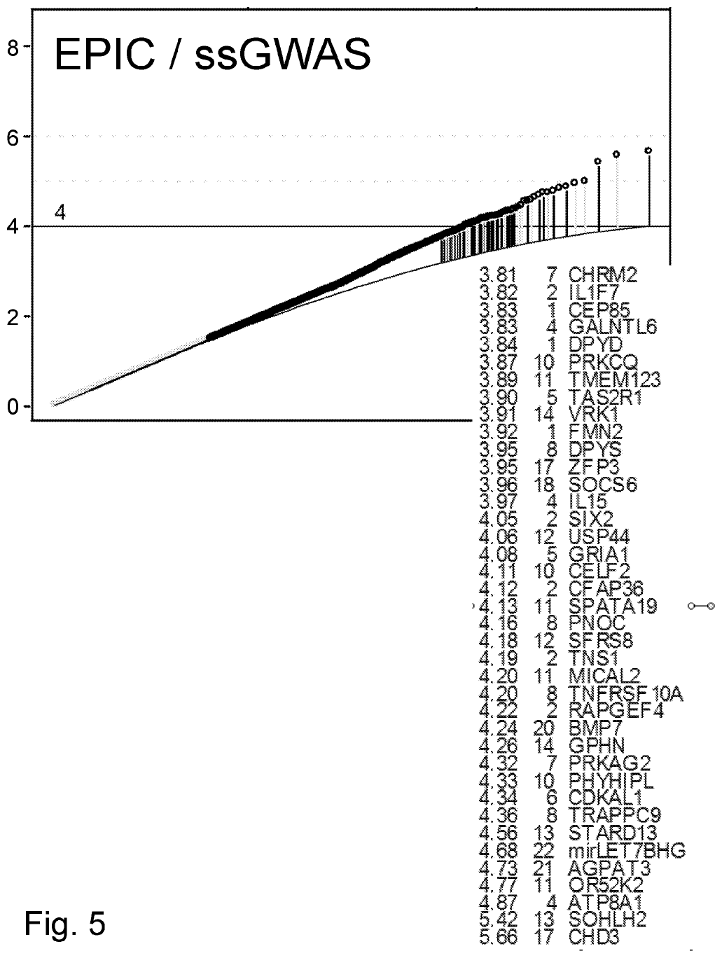

Quantile-rank (QR) plots. As is customary with selection procedures, p-values were used mainly for the purpose of ranking loci. As no particular hypotheses regarding specific loci were to be confirmed, the traditional approach of exploring characteristics of the ‘QQ plot’ as decision criteria was modified and formalized. For multivariate tests of overlapping diplotypes, the straight line expected in the traditional ‘QQ plot’ under the univariate WG permutation hypothesis turns into a curve because many tests are performed per SNP. Even though the number of tests performed increases substantially, the increase in s-values shown in the QR curve compared to the QQ line (FIG. 2 vs FIG. 1, FIG. 4 vs FIG. 3, FIG. 6 vs FIG. 5) is limited, because most tests are highly dependent. (Wittkowski et al. 2014).

Whole-genome permutation bias. To estimate the expected distribution of s-values, one could average the results of repeated runs with random phenotype permutations. As each μGWAS analysis may require >100,000 hours on a grid/cloud with GPU enabled nodes, however, simulations requiring >108 replications to estimates the 1-10⁻⁷⁵ quantile may not be feasible. The estimate from WG permutations (including computationally efficient approximations) is affected by biases due to subjects being categorized based on their outcome (e.g., non-verbal vs. verbal), rather than randomly assigned to interventions (as in clinical trials), so that the groups are expected to differ systematically in aspects related to neither the condition of interest nor common ancestry factors. With binary outcomes, significant results can also not be caused by a few ‘outliers’ only, so that significance is correlated with high MAF (ssGWAS) or low skewness of the scores (μGWAS). Hence, regions with significant allelotype differences between observed phenotypes have also a larger chance to be significant among random phenotype permutations. (Wittkowski et al. 2014).

Selective chromosome permutation. The proposed use of a selected chromosome permutation approach reduces this bias. While chromosomes may differ with respect to their content of disease related and unrelated risk factors, random errors are expected to have the same distribution across all chromosomes. Hence, the above biases are reduced by excluding chromosomes containing regions of high significance when determining the permutation distribution. In particular, the endpoint of the expected distribution for each chromosome can be estimated from the loess projection to the p-values after truncation to ensure a monotone increase and a non-positive second derivative. Similarly, the endpoint of the expected distribution is estimated from the median of the limited set of, e.g., ten, chromosomes with the lowest maximum deviation of the distribution of s-values from the loess projection (Wittkowski et al. 2014).

Formal QR cut-off for deviation. The estimate of the expected distribution for each chromosome is then calculated as the loess fit of the individual chromosomes' data with a sufficient number of results at the high end replaced with the expected endpoint until the curve is curtailed to that level, unless the initial loess fit already remains below this target level. The same procedure, when applied to the WG data, yields the estimation of the WG distribution. Simulation results demonstrate the low variance of the estimates based on random permutations of the phenotypes and that their median is closely resembled by the estimate of the distribution obtained from the observed data (Wittkowski et al. 2014).

### Results

Previously known results from CGEM: Traditionally, GWAS have often identified only a small number of SNPs per study. A previous ssGWAS analysis of the CGEMS data (Hunter et al. 2007) had implicated two loci in trend analysis:

| | | | | |
|---|---|---|---|---|
| chr10: | 124,992,475 | rs10510126: | 6.15 | BUB3, (long EST not5 |
| | | | | identified) |
| chr10: | 123336180 | rs1219648: | 5.49 | FGFR2 |
| | 123341314 | rs2420946 | 5.46 | FGFR2 |

ssGWAS results: Single-SNP GWAS confirmed these findings at essentially equivalent levels of 6.20 and 5.57, respectively. Many other results had been dismissed in previous published analyses because p-values did not reach the traditional level of “fixed genome-wide significance” (typically, 7.5). From the ssGWAS QR plots (CGEM: FIG. 1, EPIC: FIG. 3, and PBCS: FIG. 5) many of the genes above the cut-off for study-specific genome-wide significance fit the paradigm of being involved in signaling at the membrane (GPCRs, Fc receptors, growth factor receptors, ion channels) or processes in the nucleus (cell cycle control, transcription, splicing) (see FIG. 22, columns Mbrn and Ncls).

muGWAS results: In muGWAS (CGEM: FIG. 2, EPIC: FIG. 4, and PBCS: FIG. 6), the proportion of genes related to membrane signaling and nuclear processes is even higher than in ssGWAS. In addition, a group of genes known to play a role in either in the phosphatidyl-inositol (PI) cycle (FIG. 7) or in endocytosis (FIG. 6) stands out.

Validation (same intragenic region): PRKCQ (chr 10), was significant by muGWAS in CGEM (mu: 6.70, ss: 3.47, “indicating study-specific genome-wide significance) and by ssGWAS in EPIC (mu: 5.26, ss: 3.87*). The same region (chr10:6,540,724-6,573,883) was implicated in both populations. In PBCS, in contrast, there was no association in this region (<2.00), consistent with the notion of different risk factors for breast cancer in different populations.

Validation (same gene): MEGF11 was implicated in both CGEM and PBCS. MEGF11 was even elevated (3.31) in EPIC. A single SNP was highly influential in either population, but it was not the same SNP (CGEM: rs189155, PBCS: rs12903880, EPIC: rs333554). All three SNPs are located in the coding region, but they are not in LD. One other SNP in MEGF11 (rs1477798) has been implicated in colorectal cancer (Cicek et al. 2012).

Validation (similar function): In a complex disease, populations may differ with respect to the risk factors that are present in each population. In particular, the proportion of risk conferred by different genes with similar function may differ and, even if the same gene is involved, risk may be associated with different SNPs.

One pair of functionally related genes stand out in ssGWAS (FIG. 22):

BMPR1B (CGEM)-BMP7 (EPIC)

Among muGWAS results (FIG. 22), there are three more pairs of functionally related genes:

ATP8B1 (CGEM)-ATP8A1 (EPIC)

MEGF11 (CGEM, PBCS)

AGPAT4 (CGEM)-AGPAT3 (EPIC)

Mutations in PI3K, PTEN, and SYNJ2 are known to be associated with breast cancer. The mechanism commonly believed to be involved is the dysregulation of the AKT/TSC/mTOR growth pathway downstream of PI(3,4,5)P₃ and PI(3,4)P₂. The results of this analysis point to three additional points where the PI cycle is involved (FIG. 7):

PI(4,5)P2 (SCARB2, UNC13C, STXBP1, SDCBP2, MEGF11, SYT 17, N4BP 3, VAV3) and

PI(3)P (NLRP4, EEAJ, RAB32), as well as

overall activation of PI (ATP8A1, ATP8B1, SLC5A3, AGPAT3, AGPAT4, ANXA4)

With the exception of CHMP7, RAPGEF4, and EEA1, all these genes have previously been shown to be associated with breast cancer (http://www.genecards.org).

The novel finding is that breast cancer risk is conferred not only by a variety of variations in

genes involved in nuclear processes causing susceptibility for cancer and

genes involved in membrane processes providing growth signals,

as well as a few specific variations connecting the two by increasing

PI(3,4,5)P₃ (loss-of-function in PTEN, gain-of-function in PI3K),

PI(3,4)P₂ (gain-of-function in SYNJ1/2 or INPPL1), or

PI(3)P (gain-of-function in INPP4B),

but by a global dysregulation of the PI cycle, including

entry of phosphatidylinositol (PI) (involving AGPAT3, AGPAT4, and SLC5A3), and

entry of phosphatidylserine/phosphatidylcholine (PS/PC) (involving ATP8A1, ATP8B1, and ANXA4),

and endocytosis as a critical component of migration and invasion. Endocytosis is known to be controlled by PI signaling, which is consistent with the genes identified in the results presented:

at the plasma membrane stage (PM, eight genes),

at the early endosome stage (EE, four genes), and

at the late endosome stage (LE, two genes).

### DISCUSSION

The approach used here differs from traditional GWAS in both the statistical method being used and the decision strategy. To address the statistical method challenges specific to GWAS, the novel approach

(a) avoids making assumptions about a particular degree of dominance.
(b) draws for the fact that both SNPs neighboring a disease locus should be in LD, unless they are separated by a recombination hotspot.
(c) can distinguish between SNPs belonging to the same tag sets, but differ in their order along the chromosome.
(d) accounts for different disease loci within the same region having similar effects and for compound heterozygosity within the statistical method (rather than through visual inspection looking for several SNPs within a region having high significance), and
(e) provides additional information (“information content”) that can be used to prioritize results.

The use of a decision rule that accounts for

(a) GWAS being non-randomized,
(b) the aim being selecting sets of genes, knowing that some must have an effect, rather than testing the hypothesis that no gene has an effect, at all,
(c) accounting for differences in MAF in estimating the expected distribution of p-values, and
(d) adjusts for tests in overlapping diplotypes being related.

The validation of μGWAS in CAE (Wittkowski et al. 2013) demonstrated the ability of μGWAS to identify genes modulating a known disease pathway, where traditional ssGWAS had identified a single SNP (in a pseudo-gene) only. The subsequent application to mutism in autism (Wittkowski et al. 2014) confirmed the ability of μGWAS to identify clusters of genes related to the same biological function in two independent populations.

By using this novel decision rule alone with traditional single-SNP GWAS, the number of “significant” genes rises from none to >20 in each of the three studies considered here. The set of genes seen in ssGWAS using the novel decision rules includes half of the genes associated with the novel PI cycling pathway. The novel non-parametric wide-locus approach then adds the other half of the genes involved in PI cycling.

The PI cycle is critical for many cellular function in eukaryotic cells, including oocyte maturation, fertilization, and embryogenesis, cell growth, cytoskeleton dynamics, membrane trafficking, and nuclear events (Shulga et al. 2010; Busa 1988). Hence, it is not surprising that the PI cycle is tightly controlled. In particular, PI(4.5)P2, PI(4)P, PI(3,4)P2, and PI(3)P are tightly regulated by both three kinases and three groups of phosphatases (Waugh 2015). Different subsets of phosphatases (FIG. 7, boxes) are counteracting the effects of the kinases, further reducing the impact any variation in a particular phosphatase might have on the system as a whole. Hence, a specific intervention modifying the state of this tightly regulated system might not suffice to achieve a sustained effect.

That “both PtdIns(3,4,5)P₃ and PtdIns(3,4)P₂ are likely required for a cell to achieve and sustain a malignant state”, has been formulated as the “two PIP hypothesis” (Kerr 2011). The results presented here suggest not only that PI(3)P is required, but shift in focus further toward to the PI cycle as a whole. This new focus has direct implications for the development of drugs.

The model of a linear (PI-PIP-PIP2-PIP3) PI system suggested inhibition of PI3K as a strategy to reduce activity along the AKT/TSC/mTOR growth pathway. Only a small proportion of patients, however, benefit from interrupting this linear pathway by blocking PI3K (Bosch et al. 2015). The limited success of wortmannin and other drugs blocking PI3K is consistent with the ability of the PI cycle to compensate not only for natural, but also for pharmaceutical disturbance at a particular point.

High levels of PI(3,4,5)P3, PI(3,4)P2, and PI(3)P all are known to correlate with a negative outcome of cancers. “Altered abundance of phosphatidyl inositides (PIs) is a feature of cancer. Various PIs mark the identity of diverse membranes in normal and malignant cells” (Sengelaub et al. 2015). The model of a PI cycle tightly regulated around the PI(4,5)P2-PI(4)P-PI(3,4)P₂-PI(3)P pathway suggests overall downregulation of PI activity as a more successful strategy to correct for excessive activation involving the PI cycle than blocking individual or pairs of kinases or phosphatases.

Neither breast nor prostate cancer per se are lethal; it is metastases spreading to other organs that cause cancer-related death. As seen in treatments involving cytotoxic drugs, reducing cell growth, in general, often causes side-effects (nausea, loss-of-hair, . . . ), without necessarily reducing the risk of metastases, because growth and metastasis may be regulated by different pathways.

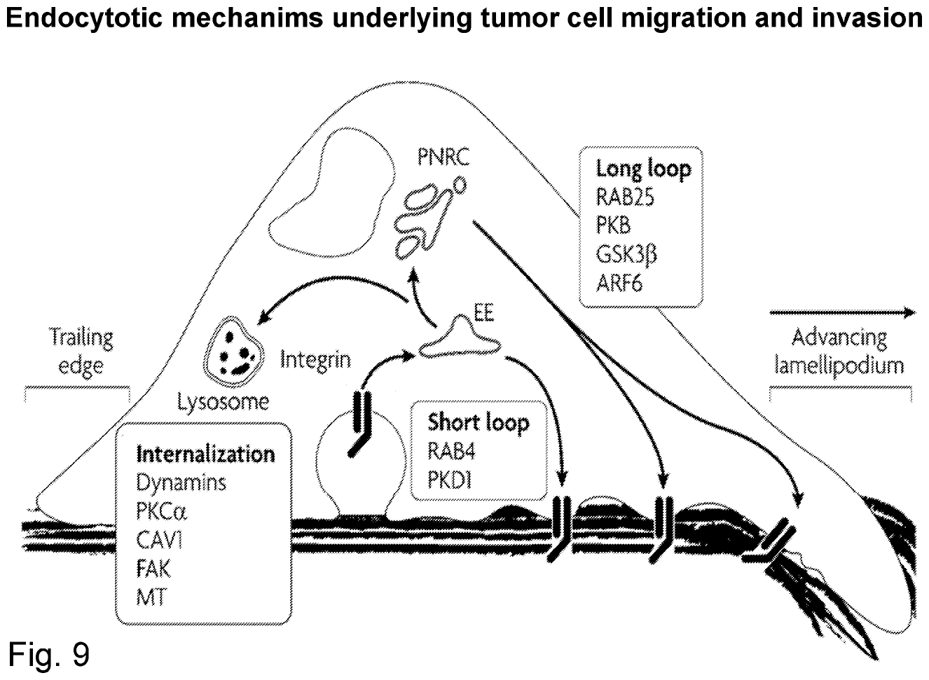

To metastasize, “tumour cells must develop motile and invasive phenotypes.” Endocytosis is known to be required for cell migration (Mellman et al. 2013). As “defective vesicular trafficking of growth factor receptors, as well as unbalanced recycling of integrin- and cadherin-based adhesion complexes, has emerged in the past 5 years as a multifaceted hallmark”, “derail[ing] endocytosis” has been suggested as a strategy to prevent metastases in cancer (Mosesson et al. 2008). “Activation of signal transduction pathways associated with endocytic trafficking (FIG. 9) is critical for tumor cell migration. As a consequence, selective targeting endocytic trafficking and signaling could potentially allow for the development of novel cancer therapeutics to prevent metastasis” (Chew et al. 2016) (see FIG. 9).

### Example 2: The Genetic Risk Factors in the PI-Cycle and Along the Endocytosis Pathway are Known as Shared Risk Factors for “Derailed Endocytosis” in Breast Cancer and Parkinson's/Alzheimer's Disease

AD and PD are known to share risk factors: Aβ and α-synuclein have been hypothesized to interact (Crews et al. 2009; Tsigelny et al. 2008), and “moderate association” of AD with PD was found in a meta analysis of 14 studies conducted 1986-2010 (Feldman et al. 2014), but a meta-analysis of single-SNP summary statistics from two sets of AD and PD GWAS, each imputed to 7,815K SNPs, “resulted in no significant evidence [for SNP] loci that increase the risk of both PD and AD” (Moskvina et al. 2013). Recently, having disease with Lewis bodies (DLB) diagnosed as either PD or AD was identified as a potential confounder in these studies (Guerreiro et al. 2016; Bras et al. 2014a), and the above results suggest that lack of “significant evidence” above may have been because of ssGWAS having lower power than muGWAS for cis-epistatic effects.

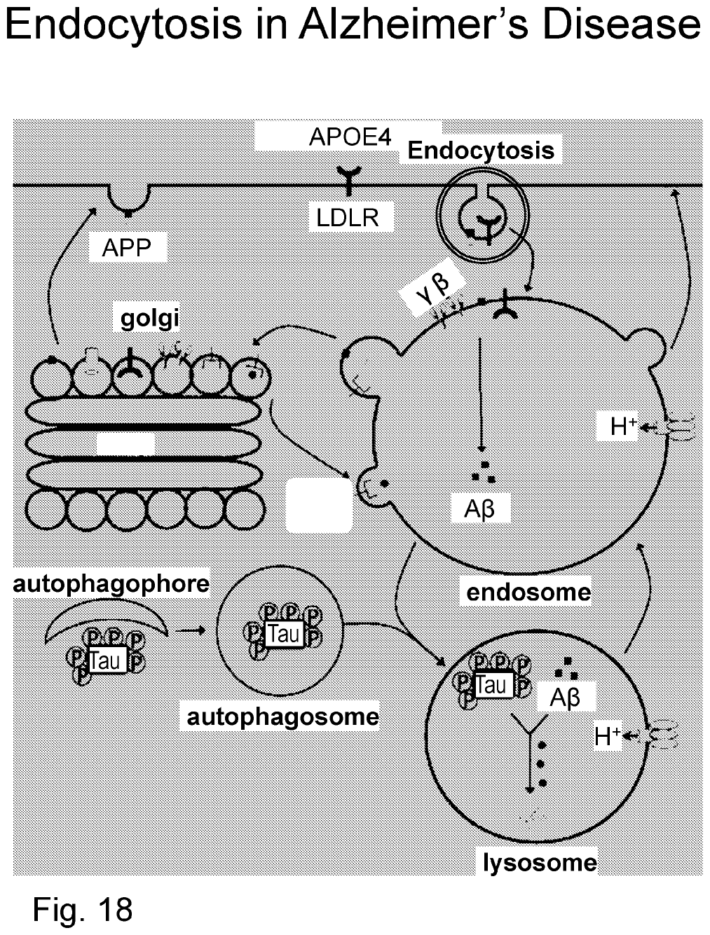

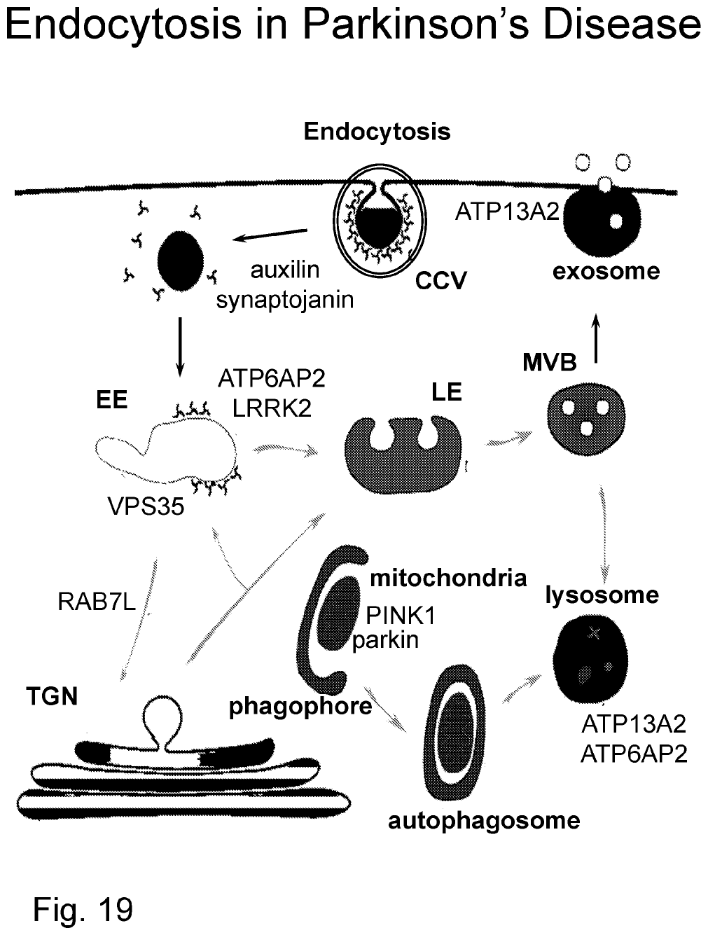

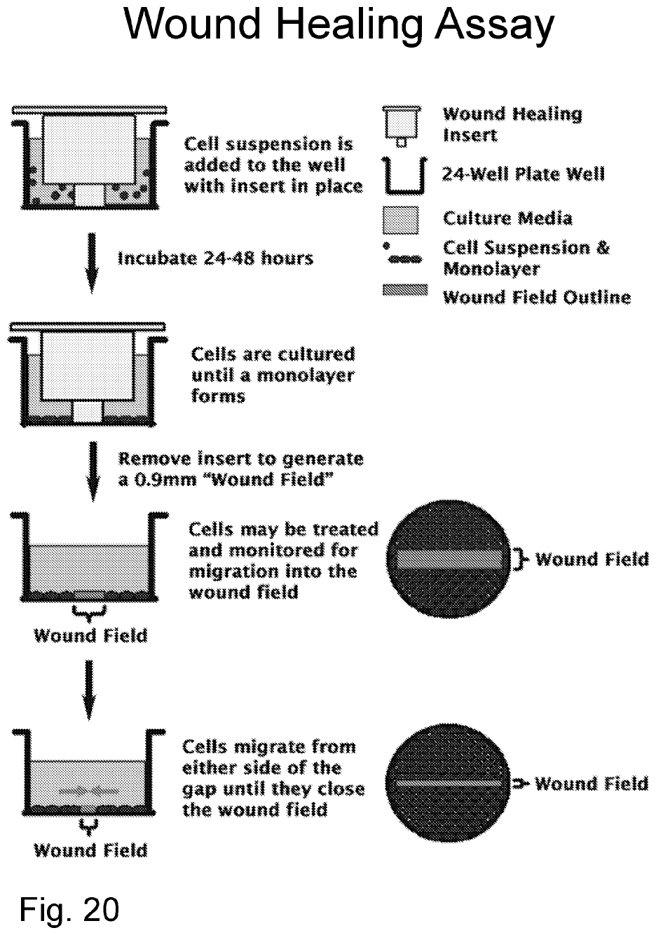

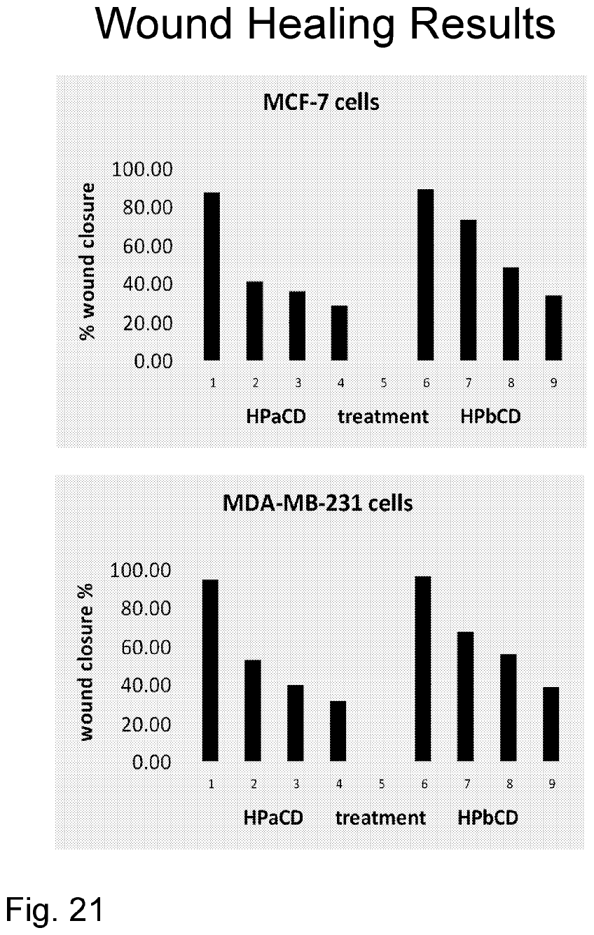

Endocytosis is a common component of the etiology of aging and neurodegenerative diseases: The term “derailed endocytosis” has been used to characterize an important component of the etiology of BC (Mosesson et al. 2008), AD (Van Dooren et al. 2014), and other “pathological conditions” (Di Fiore et al. 2014). “New reports implicate altered [vacuolar H+]-ATPase activity and lysosomal pH dysregulation in cellular aging, longevity, and adult-onset neurodegenerative diseases, including forms of [PD] and [AD]” (Colacurcio et al. 2016), see also FIG. 17, FIG. 18, and FIG. 19. In PD, “an age-related pathological depletion of functional endosomes may increase the susceptibility to stochastic molecular defects in this same pathway, which in some individuals may trigger [a] vicious circle. [ . . . ] Disease causing mutations cluster within [the endosomal] pathway and alter receptor recycling and/or α-synuclein degradation. In turn, α-synuclein accumulation [ . . . ] exacerbates defective endosomal processing by impairing the machinery involved in the sorting or fusion of endosomes” (Perrett et al. 2015). In AD, “accelerated endocytosis causes endocytic cargos to accumulate within enlarged [LEs] and impairs lysosomal functions. [ . . . ] Pathogenic endocytosis [ . . . ] could be modulated therapeutically at multiple possible targets” (Kim et al. 2016). “The underlying molecular mechanisms [in AD and PD] remain poorly understood, yet dysfunction in endocytic membrane trafficking is a recurrent theme, which may explain the neurodegenerative process” (Schreij et al. 2015).

Overlapping epidemiology and etiology of BC and AD/PD: BC has high co-occurrence with PD (Disse et al. 2016). Earlier reports that cancers reduce AD risk were linked to statistical models not accounting for competing risks (Hanson et al. 2016) and/or treatment effects of cancer drugs (Malkki 2016). Another reason for limited association between BC and AD may be that mutations may have opposite effects, such as gain-of-function in BC (and PD) and loss-of-function in AD. Overlapping genetic risk factor have already been reported. Mutations in the PD gene PSEN2 were also found in BC and AD (Cai et al. 2015). Mutations in MAPT, which encodes the AD microtubule-associated protein Tau, were found in BC (Rouzier et al. 2005) and PD (Lopez Gonzalez et al. 2016); DJ-1 was seen as elevated (Kawate et al. 2015), and the G₂₀₁₉S mutation in endosomal LRRK2 (Rivero-Rios et al. 2015) increases risk (Agalliu et al. 2015). Still, “the etiology of this link continues to be elusive” (Disse et al. 2016).

EEC is a common risk factor in BC, PD, and AD: In PD (Rivero-Rios et al. 2015) and AD (Schreij et al. 2015; Kim et al. 2016; Wang et al. 2014), endocytosis of α-synuclein (SNCA, FIG. 19) and amyloid beta precursor protein (APP, FIG. 17 and FIG. 18) and respectively, are known to be critical early steps in the etiology leading to formation of plaques. “[G]enes that influence endocytosis are overrepresented as AD risk factors [and] endocytosis-related genes are the earliest known disease-specific neuronal response in AD. They develop early in Down syndrome, a cause of early-onset AD linked to an extra copy of APP” (Kim et al. 2016).

Overlap of genetic risk factors for BC (from above and published results) and AD/PD (from published results): The vast majority of genes related to the PI-cycle and EEC genes identified in muGWAS of breast cancer (FIG. 22, column PI/EC) had already been identified in previous functional studies and gene expression studies of both BC and AD/PD (Table 4 and Table 6).

TABLE 4
PI-Cycle overlap between BC, PD, and AD. Gene: Genes identified in BC GWAS. EEC function: Known function in PI-cycle and/or EEC.
KEGG: KEGG pathway (http://www.genome.jp/kegg/pathway.html), EC: epithelial cancer (carcinoma). ND: Neurodegenerative disease.

| Gene | PI-Cycle | KEGG | EC | ND | References |
|---|---|---|---|---|---|
| ATP8A1 | Increasing extracellular PC | | | | (Farge E, Ojcius D M, et al. (1999) Am J Physiol 276: C725-33; Levano K, |
| ATP8B1 | and PS enhances endocytosis | | | | Sobocki T, et al. (2009) Glycoconj J 26: 739-48; Levano K, Punia V, et al. |
| | | | | | (2012) J Neurochem 120: 302-13) |
| | | | BC | | (da Costa A, Lenze D, et al. (2012) J Comp Pathol 146: 143-51; Sjöblom T, |
| | | | | | Jones S, et al. (2006) Science 314: 268-74) |
| | | | PC | | (Lee B H, Taylor M G, et al. (2013) Cancer Res 73: 1211-8; Sekine Y, Demosky |
| | | | | | S J, et al. (2010) Mol Cancer Res 8: 1284-94; Trasino S E, Kim Y S, et al. |
| | | | | | (2009) Mol Cancer Ther 8: 1934-45) |
| | | | | PD | (Levano K, Punia V, et al. (2012) J Neurochem 120: 302-13) |
| | | | | | ATP8A2: (Zhu X, Libby R T, et al. (2012) PLOS Genetics 8: e1002853) |
| | | | | AD | (Soderberg M, Edlund C, et al. (1992) J Neurochem 59: 1646-53) |
| | | | | | ATP8B4: (Li H, Wetten S, et al. (2008) Arch Neurol 65: 45-53) |
| ANO4 | Ca+ dependent PL scramblase | | | | (Picollo A, Malvezzi M, et al. (2015) J Mol Biol 427: 94-105) |
| | | | SC | | (Weber G F (2015) Sarcoma 2015: 839182) |
| | | | | AD | (Sherva R, Tripodis Y, et al. (2014) Alzheimers Dement 10: 45-52) |
| ABCA1 | Regulates cellular lipid efflux; | hsa02010 | | | (Hamon Y, Trompier D, et al. (2006) PLoS One 1: e120) |
| | interacts with MEGF10 | | BC | | (Zhao W, Prijic S, et al. (2016) Cancer Res 762037-49; Schimanski S, Wild P J, |
| | | | | | et al. (2010) Horm Metab Res 42: 102-9) |
| | | | PC | | (Lee B H, Taylor M G, et al. (2013) Cancer Res 73: 1211-8; Sekine Y, Demosky |
| | | | | | S J, et al. (2010) Mol Cancer Res 8: 1284-94) |
| | | | | PD | (Dong Y, Gou Y, et al. (2015) Elife 4; Pinho R, Guedes L C, et al. (2016) PLoS |
| | | | | | One 11: e0157852; Dong X, Liu T, et al. (2016) Genes & Genomics 38243-50; |
| | | | | | Loane D J, Washington P M, et al. (2011) J Neurotrauma 28: 225-36) |
| | | | | AD | (Koldamova R, Fitz N F, et al. (2014) Neurobiol Dis 72 Pt A: 13-21; Pahnke J, |
| | | | | | Langer O, et al. (2014) Neurobiol Dis 72 Pt A: 54-60; Boehm-Cagan A, Bar R, et |
| | | | | | al. (2016) PLoS One 11: e0166195; Nordestgaard L T, Tybjaerg-Hansen A, et al. |
| | | | | | (2015) Alzheimers & Dementia 11: 1430-8) |
| | | | | HD | (Valenza M, Marullo M, et al. (2015) Cell Death Differ 22: 690-702) |
| AGPAT3 | converts lysophospha- | hsa00564 | | | (Bradley R M, Marvyn P M, et al. (2015) Biochimica et Biophysica Acta (BBA) - |
| AGPAT4 | tidylinositol (LPI) into | | | | Molecular and Cell Biology of Lipids 1851: 1566-76) |
| | phosphatidylinositol (PI) | | BC | | (Sahay D, Leblanc R, et al. (2015) Oncotarget 6: 20604-20; Hopkins M M, |
| | | | | | Zhang Z, et al. (2016) J Clin Med 5) |
| | | | PC | | AGPAT6: (Gatto F, Miess H, et al. (2015) Sci Rep 5: 10738) |
| | | | | PD | (Cheng D, Jenner A M, et al. (2011) PLoS One 6: e17299) |
| | | | | AD | (Sherva R, Baldwin C T, et al. (2011) J Alzheimers Dis 23: 349-59) |
| DGKQ | Regenerates PA from | hsa00564, | | | (Sakane F, Kanoh H (1997) Int J Biochem Cell Biol 29: 1139-43) |
| | diacylglycerol (DAG) | hsa04070 | BC | | (Filigheddu N, Cutrupi S, et al. (2007) Anticancer Res 27: 1489-92) |
| | | | PC | | AGK: (Bektas M, Payne S G, et al. (2005) J Cell Biol 169: 801-11) |
| | | | | PD | (Lili C M, Roehr J T, et al. (2012) PLoS Genet 8: e1002548; Nalls M A, |
| | | | | | Pankratz N, et al. (2014) Nat Genet 46: 989-93) |
| | | | | AD | (Zhu X C, Cao L, et al. (2016) Mol Neurobiol) |
| LPPR1 | complexes with LPPR3/4/5, | | | | LPPR4: (Yu P, Agbaegbu C, et al. (2015) Journal of Cell Science 128: 3210-22) |
| | regulates PIS (CDIPT) | | | | |
| | | | | PD | (Moran L B, Duke D C, et al. (2006) Neurogenetics 7: 1-11) |
| SYNJ2 | is recruited to the nascent | hsa04070 | | | (Schmid S L, Mettlen M (2013) Nature 499: 161-2) |
| | clathrin coated pit | | BC | | (Ben-Chetrit N, Chetrit D, et al. (2015) Sci Signal 8: ra7) |
| | | | PC | | (Rossi M R, Hawthorn L, et al. (2005) Cancer Genet Cytogenet |
| | | | | | 161: 97-103) |
| | | | | PD | SYNJ1 = PARK20 |
| | | | | AD | (Koran M E, Hohman T J, et al. (2014) J Alzheimers Dis 38: 145-54) |
| PTENP1 | PI3K/PTEN and PI(3,4,5)P3 | hsa04070 | | | PTEN: (Emeux C, Ghosh S, et al. (2016) Curr Pharm Des 22: 2309-14) |
| | are involved in endocytosis/ | | BC | | (Zhang H-Y, Liang F, et al. (2013) Oncology Letters 6: 161-8) |
| | cancer | | PC | | (Pourmand G, Ziaee A A, et al. (2007) Urology journal 4: 95-100) |
| | | | | PD | PINK1: (Choubey V, Cagalinec M, et al. (2014) Autophagy 10: 1105-19) |
| | | | | AD | (Frere S, Slutsky I (2016) Nat Neurosci 19: 416-8) |
| | | | | HD | PINK1: (Khalil B, El Fissi N, et al. (2015) Cell Death Dis 6: e1617) |

Table 5 (continued): Sequences for Table 5 genes.

| Name | Entrez | Accession | SEQ ID NO. |
|---|---|---|---|
| ATP8A1 | 10396 | AF067820 | SEQ ID NO: 1 |
| 1 | mptmrrtvse irsraegyek tddvsektsl adqeevrtif inqpqltkfc nnhvstakyn | | |
| 61 | iitflprfly sqfrraansf flfiallqqi pdvsptgryt tivpllfila vaaikeiied | | |
| 121 | ikrhkadnav nkkqtqvlrn gaweivhwek vavgeivkvt ngehlpadli slsssepqam | | |
| 181 | cyietsnldg etnlkirqgl patsdikdvd slmrisgrie cespnrhlyd fvgnirldgh | | |
| 241 | gtvplgadqi llrgaqlrnt qwvhgivvyt ghdtklmqns tspplklsnv eritnvqili | | |
| 301 | lfciliamsl vcsvgsaiwn rrhsgkdwyl nlnyggasnf glnfltfiil fnnlipisll | | |
| 361 | vtlevvkftq ayfinwdldm hyeptdtaam artsnlneel gqvkyifsdk tgtltcnvmq | | |
| 421 | fkkctiagva yghvpepedy gcspdewqns qfgdektfsd ssllenlqnn hptapiicef | | |
| 481 | ltmmavchta vperegdkii yqaaspdega lvraakqlnf vftgrtpdsv iidslgqeer | | |
| 541 | yellnvleft sarkrmsviv rtpsgklrly ckgadtviyd rlaetskyke itlkhleqfa | | |
| 601 | teglrticfa vaeisesdfq ewravyqras tsvqnrllkl eesyeliekn lqllgataie | | |
| 661 | dklqdqvpet ietlmkadik iwiltgdkqe tainighsck llkknmgmiv inegsldgtr | | |
| 721 | etlsrhcttl gdalrkendf aliidgktlk yaltfgvrqy fldlalscka viccrvsplq | | |
| 781 | ksevvemvkk qvkvvtlaig dgandvsmiq tahvgvgisg neglqaanss dysiaqfkyl | | |
| 841 | knllmihgaw nynrvskcil ycfyknivly iieiwfafvn gfsgqilfer wciglynvmf | | |
| 901 | tamppltlgi ferscrkenm lkypelykts qnaldfntkv fwvhclnglf hsvilfwfpl | | |
| 961 | kalqygtafg ngktsdylll gnfvytfvvi tvclkaglet sywtwfshia iwgsialwvv | | |
| 1021 | ffgiysslwp aipmapdmsg eaamlfssgv fwmgllfipv asllldvvyk vikrtafktl | | |
| 1081 | vdevqeleak sqdpgavvlg kslteraqll knvfkknhvn lyrseslqqn llhgyafsqd | | |
| 1141 | engivsqsev iraydttkqr pdew | | |
| ATP8B1 | 5205 | AF038007 | SEQ ID NO: 2 |
| 1 | msterdsett fdedsqpnde vvpysddete delddqgsav epeqnrvnre aeenrepfrk | | |
| 61 | ectwqvkand rkyheqphfm ntkflcikes kyannaikty kynaftfipm nlfeqfkraa | | |
| 121 | nlyflallil qavpqistla wyttivpllv vlgvtaikdl vddvarhkmd keinnrtcev | | |
| 181 | ikdgrfkvak wkeiqvgdvi rlkkndfvpa dilllsssep nslcyvetae ldgetnlkfk | | |
| 241 | msleitdqyl qredtlatfd gfieceepnn rldkftgtlf wrntsfplda dkillrgcvi | | |
| 301 | rntdfchglv ifagadtkim knsgktrfkr tkidylmnym vytifvvlil lsaglaigha | | |
| 361 | yweaqvgnss wylydgeddt psyrgflifw gyiivintmv pislyvsvev irlgqshfin | | |
| 421 | wdlqmyyaek dtpakarttt lneqlgqihy ifsdktgtlt qnimtfkkcc ingqiygdhr | | |
| 481 | dasqhnhnki eqvdfswnty adgklafydh ylieqiqsgk epevrqfffl lavchtvmvd | | |
| 541 | rtdgqlnyqa aspdegalvn aarnfgfafl artqntitis elgtertynv laildfnsdr | | |
| 601 | krmsiivrtp egniklyckg adtviyerlh rmnptkqetq daldifanet lrticicyke | | |
| 661 | ieekeftewn kkfmaasvas tnrdealdkv yeeiekdlil lgataiedkl qdgvpetisk | | |
| 721 | lakadikiwv ltgdkketae nigfacellt edtticyged insllharme nqrnrggvya | | |
| 781 | kfappvqesf fppggnrali itgswlneil lekktkrnki lklkfprtee errmrtqskr | | |
| 841 | rleakkeqrq knfvdlacec saviccrvtp kqkamvvdlv krykkaitla igdgandvnm | | |
| 901 | iktahigvgi sgqegmqavm ssdysfaqfr ylqrlllvhg rwsyirmckf lryffyknfa | | |
| 961 | ftivhfwysf fngysaqtay edwfitlynv lytslpvllm glldqdvsdk lslrfpglyi | | |
| 1021 | vgqrdllfny krffvsllhg vltsmilffi plgaylqtvg qdgeapsdyq sfavtiasal | | |
| 1081 | vitvnfqigl dtsywtfvna fsifgsialy fgimfdfhsa gihvlfpsaf qftgtasnal | | |
| 1141 | rqpyiwltii lavavcllpv vairflsmti wpsesdkiqk hrkrlkaeeq wqrrqqvfrr | | |
| 1201 | gvstrrsaya fshqrgyadl issgrsirkk rspldaivad gtaeyrrtgd s | | |
| ANO4 | 121601 | AK091540 | SEQ ID NO: 3 |
| 1 | aaaaactcca ttcgaaccca tggagcagaa aaccaccgac atctactcta tgagtgctgg | | |
| 61 | gcctcctggg gcgtgtggta taaataccaa cctttggatc ttgtaaggcg gtactttgga | | |
| 121 | gagaagattg ggttatattt tgcctggttg ggctggtaca ccggcatgct cttcccagct | | |
| 181 | gccttcattg gattgtttgt ctttttgtat ggcgtcacca ctctggatca cagccaagtc | | |
| 241 | agtaaagaag tctgccaagc tacagatatc atcatgtgtc ctgtgtgtga taaatactgt | | |
| 301 | ccattcatga ggctgtcaga cagctgtgta tatgccaagg taacccacct ttttgacaat | | |
| 361 | ggagccactg tcttctttgc tgttttcatg gcagtctggg caacagtttt cctggagttt | | |
| 421 | tggaaaagac ggcgagcagt aattgcttat gactgggatt tgatagactg ggaagaagag | | |
| 481 | gaggaagaaa tacgacccca gtttgaagcc aagtattcca agaaagagcg gatgaatcca | | |
| 541 | atttctggaa agccagaacc ttatcaagca tttacagata aatgcagcag acttatcgtt | | |
| 601 | tctgcatctg gaatattttt tatgatctgc gtggtgattg ctgccgtgtt cgggatcgtc | | |
| 661 | atttaccggg tggtgactgt cagcactttc gctgccttta agtgggcgtt aatcaggaat | | |
| 721 | aactctcagg ttgcaaccac agggactgct gtgtgcatca acttctgtat cattatgttg | | |
| 781 | ctgaatgtgc tctatgaaaa agttgccctg cttctgacga atttagaaca gcctcgcaca | | |
| 841 | gagtctgagt gggagaacag cttcaccctg aaaatgtttc tttttcagtt tgtcaatctg | | |
| 901 | aacagctcca cattttacat cgcattcttc ctcggaagat ttacaggaca cccaggtgcc | | |
| 961 | tacttgaggc tgataaacag gtggagacta gaagagtgcc accctagtgg atgccttatt | | |
| 1021 | gatctgtgta tgcaaatggg tattataatg gtgctaaagc agacctggaa taatttcatg | | |
| 1081 | gaacttggct acccgttaat tcagaattgg tggactagaa gaaaagtacg acaagaacat | | |
| 1141 | ggacctgaaa ggaaaataag tttcccacaa tgggaaaagg actataacct tcagccgatg | | |
| 1201 | aatgcctatg gactcttcga tgaatactta gaaatgattc ttcagtttgg attcacaact | | |
| 1261 | atctttgtgg cagcttttcc cctagcacca cttctggcct tactgaataa cataattgaa | | |
| 1321 | attcgacttg atgcttacaa atttgtcaca cagtggagga gacctttagc ttcaagggcc | | |
| 1381 | aaagacatag gaatttggta tggaattctt gaaggcattg gaattctctc tgttatcaca | | |
| 1441 | aatgcatttg tcatagcgat aacatctgac tttatccctc gcttggtgta tgcttataag | | |
| 1501 | tatggacctt gtgcaggcca aggagaagct gggcaaaagt gcatggttgg ctatgtgaat | | |
| 1561 | gccagcttgt ctgtatttcg aatttctgac tttgagaacc gatctgagcc tgaatctgat | | |
| 1621 | ggcagtgagt tctcggggac tcctcttaag tactgcagat accgggacta ccgtgacccg | | |
| 1681 | cctcattcac tggtgcccta tggctacaca ctgcagtttt ggcatgtcct agctgctcga | | |
| 1741 | ttagctttta tcattgtctt tgagcacctc gtgttttgta taaagcacct catttcgtat | | |
| 1801 | ctgatcccag acctcccaaa agacctaagg gatcgaatga gaagagagaa gtacttgatt | | |
| 1861 | caggagatga tgtatgaagc agaactggaa cgtctccaga aggaacgaaa ggagaggaag | | |
| 1921 | aagaatggaa aagcacacca caacgagtgg ccgtgaccat aaaatagtcc ctttccaggc | | |
| 1981 | caaggacctg aattctgttt acttcttctg gctgtgcaaa agcacactca agtgaatgac | | |
| 2041 | taaaaatgca accacagtgc atgttgcaga taccggcggc cgcaggaggg gcagcatcca | | |
| 2101 | gtagaggact ggcgttggag tcacactgct gtgaaatcac gttgcagtcc agcgcacaat | | |
| 2161 | tgctatctat ccatagacca ttcttgacca agcaagcatg cacattatgg gcagttacat | | |
| 2221 | tctcaagttt ttaaaatcaa ggggaacttg tatactgggc ctgtttttca gcctgtttgc | | |
| 2281 | tacctttttt gcattctatc ccatgtgaat tttacagaca ctgggctaaa aagggtattc | | |
| 2341 | agacacatgg acacacattc ctagaatgtc atcatatggt cctaattcca tgtcaccaag | | |
| 2401 | aacacagaca agaccctgtt tacaactttt tctttccttt tttttaattt tagacctttc | | |
| 2461 | tgagaagatt attatatatg acatatctat agctatgtgt atggccatag atgtatttct | | |
| 2521 | gtgtgtacat atgtatagtc atgtattcct gcatatgtac atacaaatac agagatatat | | |
| 2581 | aaagtacata gaaattcctt acttgtaaat agccaaaaag tactgacatg agtgaatttt | | |
| 2641 | cacatttaaa tagtcatcaa tatgaagcca tgattaatgc ttgtataatg tgatgcaata | | |
| 2701 | aaatttaaaa taaatttctg cacatggaat attttc | | |
| ABCA1 | 19 | AAF86276 | SEQ ID NO: 4 |
| 1 | macwpqlrll lwknitfrrr qtcqllleva wplfiflili svrlsyppye qhechfpnka | | |
| 61 | mpsagtlpwv qgiicnannp cfryptpgea pgvvgnfnks ivarlfsdar rlllysqkdt | | |
| 121 | smkdmrkvlr tlqqikksss nlklqdflvd netfsgflyh nlslpkstvd kmlradvilh | | |
| 181 | kvflqgyqlh ltslcngsks eemiqlgdqe vselcglpre klaaaervlr snmdilkpil | | |
| 241 | rtlnstspfp skelaeatkt llhslgtlaq elfsmrswsd mrqevmfltn vnssssstqi | | |
| 301 | yqaysrivcg hpeggglkik slnwyednny kalfggngte edaetfydns ttpycndlmk | | |
| 361 | nlessplsri iwkalkpllv gkilytpdtp atrqvmaevn ktfqelavfh dlegmweels | | |
| 421 | pkiwtfmens qemdlvrmll dsrdndhfwe qqldgldwta qdivaflakh pedvqssngs | | |
| 481 | vytwreafne tnqairtisr fmecvnlnkl epiatevwli nksmellder kfwagivftg | | |
| 541 | itpgsielph hvkykirmdi dnvertnkik dgywdpgpra dpfedmryvw ggfaylqdvv | | |
| 601 | eqaiirvltg tekktgvymq qmpypcyvdd iflrvmsrsm plfmtlawiy svaviikgiv | | |
| 661 | yekearlket mrimgldnsi lwfswfissl ipllvsagll vvilklgnll pysdpsvvfv | | |
| 721 | flsvfavvti lqcflistlf sranlaaacg giiyftlylp yvlcvawqdy vgftlkifax | | |
| 781 | llspvafgfg ceyfalfeeq gigvqwdnlf espveedgfn lttsysmmlf dtflygvmtw | | |
| 841 | yieavfpgqy giprpwyfpc tksywfgees dekshpgsnq kriseicmee epthlklgvs | | |
| 901 | iqnlvkvyrd gmkvavdgla lnfyegqits flghngagkt ttmsiltglf pptsgtayil | | |
| 961 | gkdirsemst irqnlgvcpq hnvlfdmltv eehiwfyarl kglsekhvka emeqmaldvg | | |
| 1021 | lpssklkskt sqlsggmqrk lsvalafvgg skvvildept agvdpysrrg iwelllkyrq | | |
| 1081 | grtiilsthh mdeadvlgdr iaiishgklc cvgsslflkn qlgtgyyltl vkkdvessls | | |
| 1141 | scrnssstvs ylkkedsysq sssdaglgsd hesdtltidv saisnlirkh vsearlvedi | | |
| 1201 | gheltyvlpy eaakegafve lfheiddrls dlgissygis ettleeiflk vaeesgvdae | | |
| 1261 | tsdgtlparr nrrafgdkqs clrpftedda adpndsdidp esretdllsg mdgkgsyqvk | | |
| 1321 | gwkltqqqfv allwkrllia rrsrkgffaq ivlpavfvci alvfslivpp fgkypslelq | | |
| 1381 | pwmyneqytf vsndapedtg tlellnaltk dpgfgtrcme gnpipdtpcq ageeewttap | | |
| 1441 | vpqtimdlfq ngnwtmqnps pacqcssdki kkmlpvcppg agglpppqrk qntadilqdl | | |
| 1501 | tgrnisdylv ktyvqiiaks lknkiwvnef ryggfslgvs ntqalppsqe vndaxkqmkk | | |
| 1561 | hlklakdssa drflnslgrf mtgldtrnnv kvwfnnkgwh aissflnvin nailranlqk | | |
| 1621 | genpshygit afnhpinitk qqlsevaxmt tsvdvlvsic vifamsfvpa sfvvfliqer | | |
| 1681 | vskakhlqfi sgvkpviywl snfvwdmcny vvpativiii ficfqqksyv sstnlpvlal | | |
| 1741 | llllygwsit plmypasfvf kipstayvvl tsvnlfigin gsvatfvlel ftdnklnnin | | |
| 1801 | dilksvflif phfclgrgli dmvknqamad alerfgenrf vsplswdlvg rnlfamaveg | | |
| 1861 | vvfflitvli qyrffirprp vnaklspind ededvrrerq rildgggqnd ileikeltki | | |
| 1921 | yrrkrkpavd ricvgippge cfgllgvnga gksstfkmlt gdttvtrgda flnxnsilsn | | |
| 1981 | ihevhqnmgy cpqfdaitel ltgrehveff allrgvpeke vgkvgewair klglvkygek | | |
| 2041 | yagnysggnk rklstamali ggppvvflde pttgmdpkar rflwncalsv vkegrsvvlt | | |
| 2101 | shsmeeceal ctrmaimvng rfrclgsvqh lknrfgdgyt ivvriagsnp dlkpvqdffg | | |
| 2161 | lafpgsvxke khrnmlqyql psslsslari fsilsqskkr lhiedysysq ttldqvfvnf | | |
| 2221 | akdqsdddhl kdlslhknqt vvdvavltsf lqdekvkesy v | | |
| AGPAT3 | 56894 | AF156774 | SEQ ID NO: 5 |
| 1 | tctatgaaac caacatacat ggcgtttgca tcacagttgg agtcagatgt gagcccggag | | |
| 61 | ggcaggtgtc tggcttgtcc acccggaagc cctgagggca gctgttccca ctggctctgc | | |
| 121 | tgaccttgtg ccttggacgg ctgtcctcag cgaggggccg tgcacccgct cctgagcagc | | |
| 181 | gccatgggcc tgctggcctt cctgaagacc cagttcgtgc tgcacctgct ggtcggcttt | | |
| 241 | gtcttcgtgg tgagtggtct ggtcatcaac ttcgtccagc tgtgcacgct ggcgctctgg | | |
| 301 | ccggtcagca agcagctcta ccgccgcctc aactgccgcc tcgcatactc actctggagc | | |
| 361 | caactggtca tgctgctgga gtggtggtcc tgcacggagt gtacactgtt cacggaccag | | |
| 421 | gccacggtag agcgctttgg gaaggagcac gcagtcatca tcctcaacca caacttcgag | | |
| 481 | atcgacttcc tctgtgggtg gaccatgtgt gagcgcttcg gagtgctggg gagctccaag | | |
| 541 | gtcctcgcta agaaggagct gctctacgtg cccctcatcg gctggacgtg gtactttctg | | |
| 601 | gagattgtgt tctgcaagcg gaagtgggag gaggaccggg acaccgtggt cgaagggctg | | |
| 661 | aggcgcctgt cggactaccc cgagtacatg tggtttctcc tgtactgcga ggggacgcgc | | |
| 721 | ttcacggaga ccaagcaccg cgttagcatg gaggtggcgg ctgctaaggg gcttcctgtc | | |
| 781 | ctcaagtacc acctgctgcc gcggaccaag ggcttcacca ccgcagtcaa gtgcctccgg | | |
| 841 | gggacagtcg cagctgtcta tgatgtaacc ctgaacttca gaggaaacaa gaacccgtcc | | |
| 901 | ctgctgggga tcctctacgg gaagaagtac gaggcggaca tgtgcgtgag gagatttcct | | |
| 961 | ctggaagaca tcccgctgga tgaaaaggaa gcagctcagt ggcttcataa actgtaccag | | |
| 1021 | gagaaggacg cgctccagga gatatataat cagaagggca tgtttccagg ggagcagttt | | |
| 1081 | aagcctgccc ggaggccgtg gaccctcctg aacttcctgt cctgggccac cattctcctg | | |
| 1141 | tctcccctct tcagttttgt cttgggcgtc tttgccagcg gatcacctct cctgatcctg | | |
| 1201 | actttcttgg ggtttgtggg agcagcttcc tttggagttc gcagactgat aggagtaact | | |
| 1261 | gagatagaaa aaggctccag ctacggaaac caagagttta agaaaaagga ataattaatg | | |
| 1321 | gctgtgactg aacacacgcg gccctgacgg tggtatccag ttaactcaaa accaacacac | | |
| 1381 | agagtgcagg aaaagacaat tagaaactat ttttcttatt aactggtgac taatattaac | | |
| 1441 | aaaacttgag ccaagagtaa agaattcaga aggcctgtca ggtgaagtct tcagcctccc | | |
| 1501 | acagcgcagg gtcccagcat ctccacgcgc gcccgtggga ggtgggtccg gccggagagg | | |
| 1561 | cctcccgcgg acgccgtctc tccagaactc cgcttccaag agggaccttt ggctgctttc | | |
| 1621 | tctccttaaa cttagatcaa attttaaaaa aaaaaaaaaa | | |
| AGPAT4 | 56895 | AF156776 | SEQ ID NO: 6 |
| 1 | tgaacccagc cggctccatc tcagcttctg gtttctaagt ccatgtgcca aaggctgcca | | |
| 61 | ggaaggagac gccttcctga gtcctggatc tttcttcctt ctggaaatct ttgactgtgg | | |
| 121 | gtagttattt atttctgaat aagagcgtcc acgcatcatg gacctcgcgg gactgctgaa | | |
| 181 | gtctcagttc ctgtgccacc tggtcttctg ctacgtcttt attgcctcag ggctaatcat | | |
| 241 | caacaccatt cagctcttca ctctcctcct ctggcccatt aacaagcagc tcttccggaa | | |
| 301 | gatcaactgc agactgtcct attgcatctc aagccagctg gtgatgctgc tggagtggtg | | |
| 361 | gtcgggcacg gaatgcacca tcttcacgga cccgcgcgcc tacctcaagt atgggaagga | | |
| 421 | aaatgccatc gtggttctca accacaagtt tgaaattgac tttctgtgtg gctggagcct | | |
| 481 | gtccgaacgc tttgggctgt tagggggctc caaggtcctg gccaagaaag agctggccta | | |
| 541 | tgtcccaatt atcggctgga tgtggtactt caccgagatg gtcttctgtt cgcgcaagtg | | |
| 601 | ggagcaggat cgcaagacgg ttgccaccag tttgcagcac ctccgggact accccgagaa | | |
| 661 | gtattttttc ctgattcact gtgagggcac acggttcacg gagaagaagc atgagatcag | | |
| 721 | catgcaggtg gcccgggcca aggggctgcc tcgcctcaag catcacctgt tgccacgaac | | |
| 781 | caagggcttc gccatcaccg tgaggagctt gagaaatgta gtttcagctg tatatgactg | | |
| 841 | tacactcaat ttcagaaata atgaaaatcc aacactgctg ggagtcctaa acggaaagaa | | |
| 901 | ataccatgca gatttgtatg ttaggaggat cccactggaa gacatccctg aagacgatga | | |
| 961 | cgagtgctcg gcctggctgc acaagctcta ccaggagaag gatgcctttc aggaggagta | | |
| 1021 | ctacaggacg ggcaccttcc cagagacgcc catggtgccc ccccggcggc cctggaccct | | |
| 1081 | cgtgaactgg ctgttttggg cctcgctggt gctctaccct ttcttccagt tcctggtcag | | |
| 1141 | catgatcagg agcgggtctt ccctgacgct ggccagcttc atcctcgtct tctttgtggc | | |
| 1201 | ctccgtggga gttcgatgga tgattggtgt gacggaaatt gacaagggct ctgcctacgg | | |
| 1261 | caactctgac agcaagcaga aactgaatga ctgactcagg gaggtgtcac catccgaagg | | |
| 1321 | gaaccttggg gaactggtgg cctctgcata tcctccttag tgggacacgg tgacaaaggc | | |
| 1381 | tgggtgagcc cctgctgggc acggcggaag tcacgacctc tccagccagg gagtctggtc | | |
| 1441 | tcaaggccgg atggggagga agatgttttg taatcttttt ttccccatgt gctttagtgg | | |
| 1501 | gctttggttt tctttttgtg cgagtgtgtg tgagaatggc tgtgtggtga gtgtgaactt | | |
| 1561 | tgttctgtga tcatagaaag ggtattttag gctgcagggg agggcagggc tggggaccga | | |
| 1621 | aggggacaag ttcccctttc atcctttggt gctgagtttt ctgtaaccct tggttgccag | | |
| 1681 | agataaagtg aaaagtgctt taggtgagat gactaaatta tgcctccaag aaaaaaaaat | | |
| 1741 | taaagtgctt ttctgggtca aaaaaaaaaa aaaa | | |
| DGKQ | 1609 | L38707 | SEQ ID NO: 7 |
| 1 | gggcggacct aaaggggctc gggccgctcg ggccgggaat ggcggcggcg gccgagcccg | | |
| 61 | gggcccgcgc ctggctgggc ggcggctccc cgcgccccgg cagcccggcc tgcagccccg | | |
| 121 | tgctgggctc aggaggccgc gcgcgcccgg ggccggggcc ggggccggga cgngaccgag | | |
| 181 | cgggcggcgt cagagcccgg gcccgtgccg cgccgggaca cagcttccgg aaggtgacgc | | |
| 241 | tcaccaagcc caccttctgc cacctctgct ccgacttcat ctgggggctg gccggcttcc | | |
| 301 | tgtgcgacgt ctgcaatttc atgtctcatg agaagtgcct gaagcacgtg aggatcccgt | | |
| 361 | gcacgagtgt ggcacccagc ctggtccggg ttcctgtagc ccactgcttc ggcccccggg | | |
| 421 | ggctccacaa gcgcaagttc tgtgctgtct gccgcaaggt cctggaggca ccggcgctcc | | |
| 481 | actgcgaagt gtgtgagctg cacctccacc cagactgtgt gcccttcgcc tgcagtgact | | |
| 541 | gccgccagtg ccaccaggat gggcaccagg atcacgacac ccatcaccac cactggcggg | | |
| 601 | aggggaacct gccctcggga gcgcgctgcg aggtctgcag gaagacgtgc ggctcctctg | | |
| 661 | acgtgctggc cggcgtgcgc tgcgagtggt gcggggtcca ggcgcactcc ctctgctccg | | |
| 721 | cggcactggc tcccgagtgt ggcttcgggc gtctgcgctc cctggtcctg cctcccgcgt | | |
| 781 | gcgtgcgcct tctgcccggc ggcttcagca agacgcagag cttccgcatc gtggaggccg | | |
| 841 | cggagccggg cgaggggggc gacggcgccg acgggagcgc tgccgtgggt ccaggcagag | | |
| 901 | agacacaggc aactccggag tccgggaagc aaacgctgaa gatctttgat ggcgacgacg | | |
| 961 | cggtgagaag aagccagttc cgcctcgtca cggtgtcccg cctggccggt gccgaggagg | | |
| 1021 | tgctggaggc cgcactgcgg gcccaccaca tccccgagga ccctggccac ctggagctgt | | |
| 1081 | gccggctgcc cccttcctct caggcctgtg acgcctgggc tgggggcaag gctgggagtg | | |
| 1141 | ctgtgatctc ggaggagggc agaagccccg ggtccggcga ggccacgcca gaggcctggg | | |
| 1201 | tcatccgggc tctgccgcgg gcccaggagg tcctgaagat ctaccctggc tggctcaagg | | |
| 1261 | tgggcgtggc ctacgtgtcc gtgcgagtga cccctaagag cacggctcgc tctgtggtgc | | |
| 1321 | tggaggtcct gccgctgctc ggccgccagg ccgagagtcc cgagagcttc cagctggtgg | | |
| 1381 | aggtggcgat gggctgcagg cacgtccagc ggacgatgct gatggacgaa cagcccctgc | | |
| 1441 | tggaccggct acaggacatc cggcagatgt ctgtgcggca ggtgagccag acgcggttct | | |
| 1501 | acgtggcaga gagcagggat gtagccccgc acgtctccct gtttgttggc ggcctgcctc | | |
| 1561 | ccggcctgtc tcccgaggag tacagcagcc tgctgcatga ggccggggct accaaagcca | | |
| 1621 | ccgtggtgtc cgtgagtcac atctactcct cccaaggcgc ggtagtgttg gacgttgcct | | |
| 1681 | gctttgcgga ggccgagcgg ctgtacatgc tgctgaagga catggctgtg cggggccggc | | |
| 1741 | tgctcactgc cctggtgctc cccgacctgc tgcacgcgaa gctgccccca gacagctgtc | | |
| 1801 | ccctccttgt gttcgtgaac cccaagagtg gaggcctcaa gggccgagac ctgctctgca | | |
| 1861 | gcttccggaa gctactgaac cctcatcagg tcttcgacct gaccaacgga ggtcctcttc | | |
| 1921 | ccgggctcca cctgttctcc caggtgccct gcttccgggt gctggtgtgt ggtggcgatg | | |
| 1981 | gcactgtggg ctgggtgctt ggcgccctgg aggagacacg gtaccgactg gcctgcccgg | | |
| 2041 | agccttctgt ggccatcctg cccctgggca cagggaatga ccttggtcga gtcctccgct | | |
| 2101 | ggggggcggg ctacagcggc gaggacccgt tctccgtact gctgtctgtg gacgaggccg | | |
| 2161 | acgccgtgct catggaccgc tggaccatcc tgctggatgc ccacgaagct ggcagtgcag | | |
| 2221 | agaacgacac ggcagacgca gagcccccca agatcgtgca gatgagtaac tactgtggca | | |
| 2281 | ttggcatcga cgcggagctg agcctggact tccaccaggc acgggaagag gagcctggca | | |
| 2341 | agttcacaag caggctgcac aacaagggtg tgtacgtgcg ggtggggctg cagaagatca | | |
| 2401 | gtcactctcg gagcctgcac aagcagatcc ggctgcaggt ggagcggcag gaggtggagc | | |
| 2461 | tgcccagtat tgaaggcctc atcttcatca acatccccag ctggggctcg ggggccgacc | | |
| 2521 | tgtggggctc cgacagcgac accaggtttg agaagccacg catggacgac gggctgctgg | | |
| 2581 | aggttgtggg cgtgacgggc gtcgtgcaca tgggccaggt ccagggtggg ctgcgctccg | | |
| 2641 | gaatccggat tgcccagggt tcctacttcc gagtcacgct cctcaaggcc accccggtgc | | |
| 2701 | aggtggacgg ggagccctgg gtccaggccc cggggcacat gatcatctca gctgctggcc | | |
| 2761 | ctaaggtgca catgctgagg aaggccaagc agaagccgag gagggccggg accaccaggg | | |
| 2821 | atgcccgggc ggatcgtgcg cctgcccctg agagcgatcc taggtagggg tggctggggc | | |
| 2881 | agcccaaggg ctcgagccat ctctgctccc gccagccttg ttttcaggtg gtctggaggc | | |
| 2941 | agctccacgt cacacagtgg ctgtcatata ttgaagttac cttcccactg gaaaaaaaat | | |
| LPPR1 | 54886 | AY304515 | SEQ ID NO: 8 |

| | |
|---|---|
| 1 | gtggctcgga ccgccgcctg aatgtacctc gctcccggga gccggacggc ccagtagggc |
| 61 | gcactggagg acgctccgct gcgggagcct ggacagtttt tgacggtgca gtcttgctat |
| 121 | atggtgtgag aaatggctgt aggaaacaac actcaacgaa gttattccat catcccgtgt |
| 181 | tttatatttg ttgagcttgt catcatggct gggacagtgc tgcttgccta ctacttcgaa |
| 241 | tgcactgaca cttttcaggt gcatatccaa ggattcttct gtcaggacgg agacttaatg |
| 301 | aagccttacc cagggacaga ggaagaaagc ttcatcaccc ctctggtgct ctattgtgtg |
| 361 | ctggctgcca ccccaactgc tattattttt attggtgaga tatccatgta tttcataaaa |
| 421 | tcaacaagag aatccctgat tgctcaggag aaaacaattc tgaccggaga atgctgttac |
| 481 | ctgaacccct tacttcgaag gatcataaga ttcacagggg tgtttgcatt tggacttttt |
| 541 | gctactgaca tttttgtaaa cgccggacaa gtggtcactg ggcacttaac gccatacttc |
| 601 | ctgactgtgt gcaagccaaa ctacaccagt gcagactgcc aagcgcacca ccagtttata |
| 661 | aacaatggga acatttgtac tggggacctg gaagtgatag aaaaggctcg gagatccttt |
| 721 | ccctccaaac acgctgctct gagcatttac tccgccttat atgccacgat gtatattaca |
| 781 | agcacaatca agacgaagag cagtcgactg gccaagccgg tgctgtgcct cggaactctc |
| 841 | tgcacagcct tcctgacagg cctcaaccgg gtctctgagt atcggaacca ctgctcggac |
| 901 | gtgattgctg gtttcatcct gggcactgca gtggccctgt ttctgggaat gtgtgtggtt |
| 961 | cataacttta aaggaacgca aggatctcct tccaaaccca agcctgagga tccccgtgga |
| 1021 | gtacccctaa tggctttccc aaggatagaa agccctctgg aaaccttaag tgcacagaat |
| 1081 | cactctgcgt ccatgaccga agttacctga gacgactgat gtgtcacaag ctgtttttta |
| 1141 | aaatcatctt ccaattctat acttcaaaac acacagttgc tcaatgtcaa actgtgatga |
| 1201 | caaatattac gtttatctag ttagaagcta atgttttgta cattttttgt atgaggaagt |
| 1261 | gatgtagctt gccctgattt tttttttttt ttttggtcag ctttaatata tttatgccag |
| 1321 | aattttaaaa ccaacaaaat tttcttgttc aagcgtgcat tgaagaacca catttattca |
| 1381 | atggttgacg ttgttttgtg atatttgtac acaaattttc ttttctcagt tttataaaca |
| 1441 | cagaagtaaa tataacaatt cactttaaac ttttattacc acagttgctg cctcctccag |
| 1501 | aatttttgaa ttttaataaa aggcaaactt ttgagctgca ggaaggacaa tgttggttaa |
| 1561 | taataaatct caaagtcaat tgtagaaaaa aaattgtctt caaaaagaat gttgcactct |
| 1621 | gatctcttaa caaattgtta cgttcaaagt ttaaagtgat atattaacaa agtcacctag |
| 1681 | ttatacaaac aattgtcaga gaattctgga tttggagggt attggggtta tatgattctt |
| 1741 | tcttagataa tggcctctac taaataactc aagatctttc tggaatgtct tctggcaggc |
| 1801 | aggtgccact gtcagctttt ctccaaaaag cagccaacat cagcctcccc tgtcaactca |
| 1861 | acagttttgt atctcatatt atatggactt tatatgaaaa tgaatatttt acagtttgca |
| 1921 | cagtattatt ttacagaaaa ggaatcagag aatctacaac atagggcccc agaacaacag |
| 1981 | tttcactttg tggcttttaa ttattctaga attttaactg catctcattt ttctagcatg |
| 2041 | gtgagaacta atatgtaact cctttgattg aaggagctct tttgtccgta cctatcagaa |
| 2101 | tgttttcttg acacttccat gttggctctt ctcagctttt tttgtacata tttttttttt |
| 2161 | ctaaagagaa gaaaaagtta tcacaaaatg taaaaaaaga aaaaaaaaaa aaaaa |

Table 5: EEC overlap between BC and PD/AD. (see Table 4 for legend)

| Gene | EEC Function | KEGG | Ë\ddot{C} | ND | References |
|---|---|---|---|---|---|
| ASTN2 | regulates trafficking of ASTN1, during | hsa04144 | | | (Wilson PM, Fryer RH, et al. (2010) The Journal of Neuroscience |
| | early clathrin-dependent endocytosis; | | | | 30:8529-40; Solecki DJ (2012) Curr Opin Neurobiol 22:791-8) |
| | binds AP-2z | | BC | | (Kawauchi T (2012) Int J Mol Sci 13:4564-90) |
| | | | | AD | (Wang KS, Tonarelli S, et al. (2015) J Neural Transm (Vienna) |
| | | | | | 122:701-8) |
| | | | | ID | ASTN1: (Anazi S, Maddirevula S, et al. (2016) Mol Psychiatry) |
| TNS1 | controls cell polarization, migration, and | | | | (Rainero E, Howe JD, et al. (2015) Cell Rep 10:398-413; McCleverty |
| | invasion | | | | CJ, Lin DC, et al. (2007) Protein Science: A Publication of the Protein |
| | binds a5b1 integrin during endocytosis | | | | Society 16:1223-9; Burghel GJ, Lin W-Y, et al. (2013) PLoS One |
| | | | | | 8:e83859) |
| | | | | BC | (Hall EH, Daugherty AE, et al. (2009) J Biol Chem 284:34713-22) |
| MEGF11 | In C. elegans, DYN-1 (DNM1) depends on | hsa04144 | | | CED-1: (Shen Q, He B, et al. (2013) Development 140:3230-43) |
| | the function of CED-1 (MEGF10/11) | hsa04721 | | AD | MEGF10: (Sherva R, Tripodis Y, et al. (2014) Alzheimers Dement |
| | | | | | 10:45-52; Singh TD, Park SY, et al. (2010) FEBS Lett 584:3936-42) |
| SDCBP2 | Syndecans bind PI (4,5) P2 and are involved | | | | (Baietti MF, Zhang Z, et al. (2012) Nat Cell Biol 14:677-85; Hurley JH, |
| | in both endo- and exocytosis. | | | | Odorizzi G (2012) Nat Cell Biol 14:654-5) |
| | | | BC | | (Yang Y, Hong Q, et al. (2013) Breast Cancer Res 15:R50) |
| | | | | PD | (Tomlinson PR, Zheng Y, et al. (2015) Ann Clin Transl Neurol 2:353- |
| | | | | | 61) |
| | | | | AD | (Leonova EI, Galzitskaya OV (2015) Adv Exp Med Biol 855:241-58) |
| N4BP3 | NEDD4 controls growth factor receptor | hsa04144 | | | (Persaud A, Alberts P, et al. (2011) EMBO J 30:3259-73; Jung S, Li |
| | endocytosis (NEDD9 expression is assoc. | | | | C, et al. (2013) Int J Oncol 43:1587-95) |
| | with BC metastasis) | | BC | | (Jung S, Li C, et al. (2013) Int J Oncol 43:1587-95; Minn AJ, Gupta |
| | | | | | GP, et al. (2005) Nature 436:518-24; Liao CJ, Chi HC, et al. (2015) |
| | | | | | Oncotarget 6:9341-54) |
| | | | | PD | (Perrett RM, Alexopoulou Z, et al. (2015) Mol Cell Neurosci 66:21-8) |
| | | | | AD | (Rodrigues EM, Scudder SL, et al. (2016) J Neurosci 36:1590-5; |
| | | | | | Salminen A, Kaarniranta K, et al. (2013) Prog Neurobiol 106-107:33- |
| | | | | | 54) |
| SYNJ2 | is recruited to the nascent clathrin | hsa04070 | BC | PD | see (Table 4) |
| | coated pit | | | | |
| NLRP4 | and NLRP3 associate with BECN1, a | | | | (Jounai N, Kobiyama K, et al. (2011) J Immunol 186:1646-55; Zhang |
| | component of the PI3K complex | | | | Y, Sauler M, et al. (2014) The Journal of Immunology 192:5296-304; |
| | that mediates vesicle trafficking | | | | Rohatgi RA, Shaw LM (2016) Mol Cell Oncol 3) |
| | | | BC | | (Zhiyu W, Wang N, et al. (2016) Oncotarget 7:50766) |
| | | | | PD | (Choubey V, Cagalinec M, et al. (2014) Autophagy 10:1105-19; Wang |
| | | | | | JD, Cao YL, et al. (2015) Autophagy 11:2057-73) |
| | | | | AD | (Antonell A, Llado A, et al. (2015) Mol Neurobiol; Swaminathan G, |
| | | | | | Zhu W, et al. (2016) Autophagy 12:2404-19) |
| PTENP1 | PI3K/PTEN and PI(3,4,5)P3 are involved in | hsa04070 | | | see (Table 4) |
| | endocytosis and cancer | | | | |
| VAV3 | VAV promote BCR endocytosis | hsa04666 | | | (lnabe K, Ishiai M, et al. (2002) J Exp Med 195:189-200; Malhotra S, |
| | | | | | Kovats S, et al. (2009) The Journal of Biological Chemistry |
| | | | | | 284:36202-12) |
| | | | BC | | (Chen XIN, Chen SI, et al. (2015) Oncology Letters 9:2143-8) |
| | | | | PD | (Moran LB, Duke DC, et al. (2006) Neurogenetics 7:1-11) |
| | | | | AD | (Wilkinson BL, Cramer PE, et al. (2012) Neurobiology of Aging |
| | | | | | 33:197.e21-.e32) |
| PDE4D* | Binds ARRB2 (fast recycling) | hsa04144 | | | (Haddad SA, Ruiz-Narvaez EA, et al. (2016) Carcinogenesis) |
| | | | BC | | (Lin D-C, Xu L, et al. (2013) Proc Natl Acad Sci USA 110:6109-14) |
| | | | | PD | (Yang L, Calingasan NY, et al. (2008) Exp Neurol 211:311-4) |
| | | | | AD | (Gurney ME, D'Amato EC, et al. (2015) Neurotherapeutics 12:49-56) |
| EEA1 | binds to early endosomes in a Rab5 and | hsa04144 | | | (Pfeffer SR (1999) Nat Cell Biol 1:E145-E7) |
| | PI(3)P dependent manner. | | | | |
| | | | | PD | (Walter J, Fluhrer R, et al. (2001) J Biol Chem 276:14634-41) |
| | | | | AD | (Armstrong A, Mattsson N, et al. (2014) Neuromolecular Med 16:150- |
| | | | | | 60) |
| RAB32 | RAB32/RAB38 interact AP-3 and with | hsa05012 | | | (Hesketh GG, Perez-Dorado 1, et al. (2014) Dev Cell 29:591-606; |
| | LRRK2 (PA7-8) | | | | Waschbusch D, Michels H, et al. (2014) PLoS One 9:e111632; |
| | | | | | Bultema JJ, Ambrosio AL, et al. (2012) J Biol Chem 287:19550-63) |
| | | | BC | | (Agalliu I, San Luciano M, et al. (2015) JAMA Neurol 72:58-65) |
| | | | | PD | (Fukuda M (2016) Traffic 17:709-19) |
| | | | | AD | (Fukuda M (2016) Traffic 17:709-19) |
| SNX32* | Sorting Nexin (late endosome), SNX-BAR | hsa04144 | | | (Wang X, Huang T, et al. (2014) Molecular Neurodegeneration 9:1-9; |
| | retromer with other Vps17 orthologs | | | | van Weering JR, Verkade P, et al. (2012) Traffic 13:94-107; Zhang |
| | SNX5/SNX6 interacts with VP535 | | | | QY, Tan MS, et al. (2015) Mol Neurobiol) |
| | | | BC | | (Rivera J, Megias D, et al. (2010) J Cell Biochem 111:1464-72) |
| | | | | PD | (Small SA, Petsko GA (2015) Nat Rev Neurosci 16:126-32) |
| | | | | AD | (Reitz C (2012) Future Neurol 7:423-31) |
| SCARB2 | required for maintenance of endo- and | hsa04142 | | | (Gonzalez A, Valeiras M, et al. (2014) Mol Genet Metab 111:84-91) |
| | lysosomes, located in limiting membranes | | BC | | (Nishimura Y, Yoshioka K, et al. (2006) Histochem Cell Biol 126:627- |
| | | | | | 38; Nishimura Y, ltoh K, et al. (2003) Pathol Oncol Res 9:83-95) |
| | | | | PD | (Alcalay RN, Levy OA, et al. (2016) NPJ Parkinsons Dis 2) |
| | | | | AD | (Shimizu E, Kawahara K, et al. (2008) J Immunol 181:6503-13; Bras |
| | | | | | J, Guerreiro R, et al. (2014b) Hum Mol Genet 23:6139-46) |
| GLB1 | Galactosidase Beta, related to Galectin 3 | | | | (Ahmed H, AlSadek DM (2015) Clin Med Insights Oncol 9:113-21) |
| | (LGALS3) | | BC | | (O'Reilly EA, Gubbins L, et al. (2015) BBA Clin 3:257-75) |
| | | | | PD | (van Dijk KD, Persichetti E, et al. (2013) Mov Disord 28:747-54) |
| | | | | AD | (Tiribuzi R, Orlacchio A, et al. (2011) J Alzheimers Dis 24:785-97) |
| RAPGEF4 | GEF for RAB1A/1B/2A; involved in | | | | (Parnell E, Palmer TM, et al. (2015) Trends Pharmacol Sci 36:203-14; |
| | excocytosis through RIMS2 | | | | Almahariq M, Tsalkova T, et al. (2013) Mol Pharmacol 83:122-8) |
| | | | BC | | (Jiang HL, Sun HF, et al. (2015) Oncotarget 6:16352-65) |
| | | | | PD | (Winslow AR, Chen CW, et al. (2010) J Cell Biol 190:1023-37) |
| | | | | AD | (Puthiyedth N, Riveros C, et al. (2016) PLoS One 11:e0152342; |
| | | | | | Bereczki E, Francis PT, et al. (2016) Alzheimers Dement) |
| UNC13C | Interacting with each other and with | | | | (Betz A, Okamoto M, et al. (1997) J Biol Chem 272:2520-6; Martin TF |
| STXBP1 | PI(4,5)P2. Involved in docking/priming in | hsa04721 | | | (2015) Biochim Biophys Acta 1851:785-93) |
| (MUNC18) | exocytosis | | BC | | (Fernandez-Nogueira P, Bragado P, et al. (2016) Oncotarget 7:5313- |
| | | | | | 26) |
| | | | | PD | (Keogh MJ, Daud D, et al. (2015) Neurogenetics 16:65-7; Campbell |
| | | | | | IM, Yatsenko SA, et al. (2012) Genet Med 14:868-76) |
| | | | | AD | (Leonova EI, Galzitskaya OV (2015) Adv Exp Med Biol 855:241-58; |
| | | | | | Miller JA, Woltjer RL, et al. (2013) Genome Med 5:48; Takahashi M, |
| | | | | | lseki E, et al. (2000) J Neurol Sci 172:63-9; Law C, Schaan Profes M, |
| | | | | | et al. (2016) J Neurosci 36:561-76) |
| STXBP4* | Prevents interaction between STX4 and | hsa04130, | | | (Zhang QY, Tan MS, et al. (2015) Mol Neurobiol) |
| | VAMP2 | hsa04721 | BC | | (Antoniou AC, Beesley J, et al. (2010) Cancer Res 70:9742-54; Day |
| | | | | | P, Riggs KA, et al. (2011) Int J Oncol 39:863-71) |
| | | | | PD | (Diao J, Burre J, et al. (2013) Elife 2:e00592) |
| | | | | AD | (Russell CL, Semerdjieva S, et al. (2012) PLoS One 7:e43201) |
| ANXA4 | Forms exocytotic complexes with SYT1 | hsa04721 | | | (Lizarbe MA, Barrasa JI, et al. (2013) Int J Mol Sci 14:2652-83; |
| | and the RAB3A effector RPH3A. | | | | Willshaw A, Grant K, et al. (2004) FEBS Lett 559:13-21) |
| | | | BC | | (Wei B, Guo C, et al. (2015) Clin Chim Acta 447:72-8; Yao H, Sun C, |
| | | | | | et al. (2016) Front Biosci (Landmark Ed) 21:949-57) |
| | | | | PD | (Matigian N, Abrahamsen G, et al. (2010) Disease Models & |
| | | | | | Mechanisms 3:785-98) |
| | | | | AD | (Kuzuya A, Zoltowska KM, et al. (2016) BMC Biol 14:25; Tan MG, |
| | | | | | Lee C, et al. (2014) Neurochem Int 64:29-36) |
| | | | | HD | SYT1: (Valencia A, Sapp E, et al. (2013) Journal of Huntington's |
| | | | | | disease 2:459-75) |
| SYT17 | ″B/K protein may play a role in exocytosis″ | | | | (Chin H, Choi SH, et al. (2006) Exp Mol Med 38:144-52; Fukuda M |
| | | | | | (2013) Madame Curie Regulated Database +Internet+) |
| | | | BC | | (Weng L, Ziliak D, et al. (2013) Annals of Oncology) |
| | | | | AD | (Gautam V, D'Avanzo C, et al. (2015) Mol Neurodegener 10:31) |
| PARK2 | ″Loss of parkin promotes...endocytosis by | hsa04141 | | | (Cha SH, Choi YR, et al. (2015) Mol Neurodegener 10:63; Ahmed |
| | accumulating CAV1″; PARK2 binds AP-2 | | | | MR, Zhan X, et al. (2011) Biochemistry 50:3749-63) |
| | | | | | via arrestiri |
| | | | BC | | (Wang H, Liu B, et al. (2009) J Pathol 218:76-85) |
| | | | | PD | (Feng DD, Cal W, et al. (2015) Transl Neurodegener 4:20; Kitada T, |
| | | | | | Asakawa S, et al. (1998) Nature 392:605-8) |
| | | | | AD | (Martin-Maestro P, Gargini R, et al. (2016) Hum Mol Genet 25:792- |
| | | | | | 806) |
| DNAJC1* | ER membrane protein. DNAJC (Hsp40) | hsa04141 | BC | | (Michailidou K, Hall P, et al. (2013) Nat Genet 45:353-61, 61e1-2; |
| | controls release of proteins via HSPA5 | | | | Chen C-L, Hou W-H, et al. (2009) Journal of Cell Science 122:1863- |
| | (BiP, GRP78); DNAJC13 interacts with | | | | 71) |
| | SNX-BAR | | | PD | DNAJC6/13: (Seaman M, Freeman CL (2014) Commun lntegr Biol |
| | | | | | 7:e29483; Vilarino-Guell C, Rajput A, et al. (2014) Hum Mol Genet |
| | | | | | 23:1794-801) |
| | | | | AD | (Hsu WC, Wang HK, et al. (2008) J Neural Transm (Vienna) |
| | | | | | 115:1537-43) |

*from previous GWAS. Underlined: functionally related genes identified in the literature.

The results presented here show that the number of combinations of genes involved in different patients is too large for the goal of “targeting endocytosis” to be likely achieved by selectively targeting individual or even pairs of phosphatases or kinases (Table 5) or by targeting individual genes regulated by the PI cycle (Table 6). As discussed above, the PI cycle is designed to compensate for dysregulation of individual kinases or phosphatases. “Decreasing levels of PA” by siRNA directed toward DGKQ or PLD and use of several inhibitors (including Wortmannin) and activators aiming at “increasing intracellular levels of PIP and/or PIP2” were proposed based on the linear PI-PIP-PIP2-PIP3 model and the effect of Aβ on PA and PIPs (U.S. Pat. No. 8,288,378).

The breadth of genes involved in entry of PI and PS/PC into the PI cycle suggests a different strategy as more effective. Extraction of phospholipids reduces the intracellular concentration of phospholipids, which are known to regulate endocytosis during ligand binding (PI(4,5)P2), pit-formation (PIMP), vesicle formation (PI(3,4)P2), fusion to an early endosome (PI(3)P), and sorting into cell organelles, including the lysosomes, which are involved in NPC1 (PI(3,5)P2). “Activation of signal transduction pathways associated with endocytic trafficking is critical for tumor cell migration [and disease progression in PD/AD]. As a consequence . . . targeting endocytic trafficking and signaling could potentially allow for the development of novel cancer therapeutics to prevent metastasis [and anti-aging therapeutics to prevent PD and AD]” (Chew et al. 2016).

For instance, FAK (integrin-mediated focal adhesion kinase) is overexpressed and activated in tumors, but rarely mutated (Alanko et al. 2016). FAK inhibitors have been shown to decrease tumor growth, metastasis, and angiogenesis in mice, and are in early clinical trials for non-hematologic cancers, including, but not limited to, pancreatic cancer, lung cancer, mesothelioma, and ovarian cancer, with mixed results (clinicaltrials.gov). Regulating endocytosis of integrins provides an alternative to reduce the activity of integrin-mediated focal adhesion kinase, either alone or in combination with immunotherapy (Symeonides et al. 2017).

### Example 3: A-Cyclodextrin Restores “Derailed Endocytosis” in BC and PD/AD

The invention provides α-cyclodextrin and analogues and derivatives thereof as non-limiting examples of compounds that may be useful for treating age-related conditions including but not limited to conditions involving “derailed endocytosis”, a “hallmark of cancer” (Mitra et al. 2012), also seen in neurodevelopmental diseases (Van Dooren et al. 2014; Rivero-Rios et al. 2015). In some embodiment of the present invention α-cyclodextrins are used for treating age-related conditions, such as cancers (including, but not limited, to breast or prostate cancer) and neurodegenerative diseases (including, but not limited to, PD or AD). CDs lower the amount of PIPs available without directly interfering with their distribution.

A plethora of studies have investigated the effect of methyl-β-cyclodextrin (MβCD) in vitro. MβCD suppressed invasion activity in three H7 Lewis lung cancer cell lines and highly metastatic cell lines had more bl integrin (Zhang et al. 2006) and BC and prostate cancer cell lines were more sensitive to MβCD-induced cell death than their normal counterparts (Li et al. (2006; quiz 404-5).

In particular, MβCD treatment induced a substantial decrease (40%) in activity of BC resistance protein (BCRP/ABCG2) (Storch et al. 2007), which transports PS and PC analogues (Daleke 2007). In subsequent functional studies, MβCD inhibited spheroid migration and invasion of MDA-MB-241 and ZR751 BC cells (Raghu et al. 2010), and also endocytosis (Palaniyandi et al. 2012) and migration (Guerra et al. 2016) of MCF7 BC cells. MβCD was more toxic for invasive than for non-invasive urothelial cancer cells (Resnik et al. 2015), interfered with RTK-[PIP2]-PI3K-[PIP3]-AKT signaling in HeLa cells (Yamaguchi et al. 2015), and inhibited the growth of leukemia cell lines (Yokoo et al. 2015).

The relevance of the above in vitro findings was confirmed by several in vivo studies. MβCD had higher concentration in tumor than in other cells (except kidney and liver) and was effective in a mouse model of BC (Grosse et al. 1998), reduced the number of lung metastases in mice implanted with H7-O Lewis lung cancer cells (Zhang et al. (2006), and inhibited growth of primary effusion lymphoma (PEL) in mice (Gotoh et al. 2014). HPβCD was necessary in triple combination treatment for tumor regression in mice implanted with renal cancer cells (Yamaguchi et al. 2015), and prolonged survival in leukemia mouse models (Yokoo et al. 2015).

In mouse models of cancer, (M-)β-cyclodextrins have found to increase the effectiveness of anti-tumor drugs, such as curcumin in a lung cancer mouse model (Rocks et al. 2012) and of raloxifen in a chemically induced tumor mouse model (Agardan et al. 2015). As cholesterol has been found elevated in several cancers, the higher efficacy of anti-tumor drugs when delivered with β-cyclodextrin as an expedient has been attributed to the ability of larger cyclodextrins to scavenge cholesterol after the drug is released. More recently, β-cyclodextrin by itself has been proposed as a drug to reduce cancer growth by scavenging cholesterol in mouse models of melanoma (Mohammad et al. 2014), and leukemia (Yokoo et al. 2015). The genetics results presented here, are consistent with cyclodextrins being effective, but replace the previous hypothesis of scavenging cholesterol (control of cancer growth) with evidence for scavenging phospholipids (control of endocytosis) as a more specific mechanism for reducing the risk of metastases.

Scavenging of cholesterol and/or binding directly to Ab or α-synuclein was also believed to be the mode of action for β-cyclodextrin in AD and PD: “HPβCD, which diminishes the pool of both cholesterol and PLs, had “neuroprotective effects [ . . . ] in a transgenic mouse model of AD [by] enhancing clearance mechanisms” (Yao et al. 2012). “Toxicity of Ab1-40/42 was reduced in rats via stereotactical injection [of βCD] into the hippocampus” (Jameson et al. 2012), an effect that was attributed to the ability of bCD to interact with Ab (Camilleri et al. 1994). Similarly, in PD, that “treatment of mice with MβCD resulted in [ . . . ] reduced accumulation of α-synuclein in neuronal cell body and synapses” (Bar-On et al. 2006) was seen as related to β-cyclodextrin to prevent aggregation of α-synuclein ex vivo (Gautam et al. 2014) via direct interaction. The results of the present invention, instead, suggest that cyclodextrins act by regulating endocytosis as a common component in the etiology, the same age-related mechanism controlled by cyclodextrins in cancer.

From the mechanism of βCD in NPC and elevated cholesterol levels seen in several cancers, including BC (Yokoo et al. 2015), βCDs were thought to reduce cancer growth in BC by lowering cholesterol levels. Early evidence that this might not be the case emerged from the study of exosomes, which play a key role in development of BC (Lowry et al. 2015; Peinado et al. 2011). Treatment of MDA-MB-231 BC cells with MβCD inhibited the internalization of exosomes containing integrins (Hoshino et al. 2015), but did so independently of cholesterol (Koumangoye et al. 2011).

While HPβCD was effective against tumors in animal models and well tolerated in most peripheral and central organ systems (Cronin et al. 2015), it was shown to carry the risk of causing permanent hearing loss in mice (Crumling et al. 2012), cats (Ward et al. 2010; Vite et al. 2015), and one human (Maarup et al. 2015). This ototoxicity is believed to be due to depriving prestin (SLC26A5) in outer hair cells of cholesterol (Takahashi et al. 2016; Kamar et al. 2012; Yamashita et al. 2015).

The role of the PIP cycle emerging from our results, however, suggests a different mechanism than scavenging of cholesterol. A different mechanism is consistent with previously reported in vivo results: CAV1 expression in BC stroma increases tumor migration and invasion (Goetz et al. 2011) and CAV1 is required for invadopodia formation specifically by BC cells, where CAV1 knockdown cannot be rescued by cholesterol (Yamaguchi et al. 2009). Growing MDA-MB-231 BC cells in lipoprotein depleted medium resulted in an 85% decrease in cell migration (Antalis et al. 2011). LPA activates the Arf6-based mesenchymal pathway for migration and invasion of renal cancer cells, which originate, like BC cells, from cells located within epithelial ductal structures (Hashimoto et al. 2016).

Scavenging phospholipids, which regulate endocytosis more specifically (by α-cyclodextrin, six starch molecules), rather than also scavenging larger cholesterol molecules (by the larger β-cyclodextrin, seven sugar molecules) avoids cholesterol-mediated side effects, including ototoxicity (Cronin et al. 2015). “The acryl chain of phospholipids fits tightly into the hydrophobic cavity of the smallest α-CD and more loosely into the larger inner space of β- and γ-CDs, whereas the side chain of cholesterol is preferably included in the β-CD cavity” (Irie et al. 1997). “Cyclodextrins partially removed phospholipids . . . with a potency of α>β>>γ. Cholesterol . . . was extracted . . . most effectively by β-cyclodextrin . . . , while [the effect] of α-cyclodextrin was negligible even at hemolytic concentrations” (Ohtani et al. 1989). “β-CD also remove proteins from erythrocyte membranes” (Irie et al. 1997; FIG. 16).

At 10 mM, α- and β-cyclodextrin reduce transferrin endocytosis by 20% and 80%, respectively, which has been interpreted as α-cyclodextrin not having “any significant effect” (Rodal et al. 1999). The results presented here, however, show that these results are consistent with a more physiologic 30% of phospholipids, rather than an extreme 95% of cholesterol released at this concentration (FIG. 16). (Ohtani Y, Irie T, et al. (1989) European Journal of Biochemistry 186:17-22). Moreover, even increasing the concentration of α-cyclodextrin above 10 mM does not increase release of phospholipids, which might disrupt vital functions, while increasing the concentration of β-cyclodextrin results in a massive increase of protein release (FIG. 16). Hence, at the same level where β-cyclodextrin interferes with vital cell function by limiting available cholesterol, α-cyclodextrin merely reduces endocytosis to normal ranges by reducing regulatory phospholipids (Ohtani et al. 1989).

“PIPs are [also] involved in . . . common neurodegenerative conditions such as Alzheimer's that are becoming more widespread as life expectancy increases” (Waugh 2015). Membrane anchored inhibitors of β-secretase have been postulated as a strategy to prevent endocytosis of APP (Rajendran et al. 2010). The results presented herein provide reduction of overall endocytosis by attenuation of PI levels via α-cyclodextrin as an alternative to inhibiting β-secretase α-cyclodextrin, which has higher affinity for Neuregulin than for APP (Ben Halima et al. 2016).

Since cyclodextrins have been successfully applied both intravenously and intrathecally, different routes of administrations can be used to prevent bone and lung metastases vs glioblastoma and neurodegenerative diseases.

### Example 4: α-CD is Safer than α-CD and More Effective in Preventing Migration of Human Tumor Cells

### Introduction

Published results have shown that β-CD inhibits human MDA-MB 231 cell migration (Guerra et al. 2016). This inhibition was attributed to the ability of β-CD to “deplete cholesterol”. β-CD, however, depletes also phospholipids. To determine whether inhibition of migration is caused by β-CD depleting cholesterol, as commonly assumed, or by β-CD depleting phospholipids, as implicated by the results of the present invention, the wound healing experiment was replicated, comparing both HPbCD (Sigmna, 389145-5G) and HPaCD (Sigma, 390690-5G) vs control in both MDA-MB 231 (CRM-HTB-26, ER−) and MCF-7 (ATCC HTB-2, ER+) human breast epithelial cell lines.

### Method

Cells were cultured in 24-well culture plates (Cytoselect CBA-120, 0.9 mm wound healing/gap closure migration assay) for 24 hours with wound healing insert in place. Cells were then treated for 2 hours with HPbCD, HPaCD, or control.

Protocol: Warm up the 24-well plate (CBA-120, Cell BioLabs Inc.) with 0.9 mm CytoSelect Wound Healing Inserts at room temperature for 10 minutes. Using sterile forceps, orient the desired number of inserts in the plate wells with their “wound field” aligned in the same direction. Create a cell suspension containing 0.5-1.0×10⁶ cells/ml in media containing 10% fetal bovine serum (FBS). Add 500 μL of cell suspension to each well by carefully inserting the pipette tip through the open end at the top of the insert. For optimal cell dispersion, add 250 μL of cell suspension to either side of the open ends at the top of the insert. Incubate cells in a cell culture incubator for 12-24 hours. Carefully remove the insert from the well to begin the wound healing assay. Use sterile forceps to grab and lift the insert slowly from the plate well. Slowly aspirate and discard the media from the wells. Wash wells with media to remove dead cells and debris. Finally, add media to wells to keep cells hydrated. Repeat wash if wells still have debris or unattached cells. When washing is complete, add media with FBS and/or compounds to continue cell culture and wound healing process. Agents that inhibit or stimulate cell migration can be added directly to the wells. Incubate cells in a cell culture incubator (2 hours) and then wash cells with PBS and then add fresh media without compounds. Incubate 12-24 hours before wash and fixing. Remove the fixation solution and add 400 μL of Cell Stain Solution to each well. Allow the stain to incubate with the cells for 15 minutes at room temperature. Aspirate and discard the solution. Carefully wash each stained well 3× with deionized water. Discard washes and allow cells to dry at room temperature. Cells that migrated into the wounded area or protruded from the border of the wound were visualized and photographed under an inverted microscope. Determine the surface area of the defined wound area: Total Surface Area=0.9 mm x length. Determine the surface area of the migrated cells into the wound area: Migrated Cell Surface Area=length of cell migration (mm)×2 x length. Percent Closure (%)=Migrated Cell Surface Area/Total Surface Area x 100.

To minimize biases, wells were arranged as shown in Table 6.

TABLE 6
24-well layout. ri: i-th replication

| | r1 | | r2 | | r3 | |
|---|---|---|---|---|---|---|
| | HPaCD | HPbCD | HPaCD | HPbCD | HPaCD | HPbCD |
| mM | 0 | 0 | 4 | 4 | 0 | 0 |
| | 1 | 1 | 2 | 2 | 1 | 1 |
| | 2 | 2 | 1 | 1 | 2 | 2 |
| | 4 | 4 | 0 | 0 | 4 | 4 |

Cell-based scratch assay. Cells were cultured in 24-well culture plates for 24 h up to 90%-100% confluence. Scratched wound lines on the upside of cultured cells were created by 200 μl yellow micropipette tip. The scratched cells were washed with PBS after removal of culture media. The cells were cultured for 2 h with 2 mM MbCD and after the removal of culture media cells were cultured for the next 2, 8, 12 or 24 h. All cell-based scratch assays were performed in the presence of the anti-mitotic reagent cytosine arabinoside (ara-c; Sigma) at a final concentration of 10⁻⁵M in order to inhibit cell proliferation and analyze only cell migration. The wound area was measured from the image taken with an Axiovert 100 microscope (Carl Zeiss, Germany) by Image J program (NIH, USA) at 3 different sites from each wound area of gaps. Three independent experiments were performed.

Other methods used are described in Guerra et al. (2016) “Membrane cholesterol depletion reduces breast tumor cell migration by a mechanism that involves non-canonical Wnt signaling and IL-10 secretion.” Translational Medicine Communications 1:3, and Okada et al. (1995) “Inhibition of Human Vascular Smooth Muscle Cell Migration and Proliferation by β-Cyclodextrin Tetradecasulfate.” The Journal of Pharmacology and Experimental Therapeutics 273(2):948-954, each of which are incorporated herein by reference in their entireties.

### Results

Results are shown in Table 7. In the absence of cyclodextrins, >80% of the wound are is closed. With CDs, wound closure is inhibited in a phospholipid-dependent manner. Extraction of cholesterol does not seem to inhibit wound closure, but has been shown to cause side-effects (ototoxicity).

TABLE 7
Wound healing results.

| | | | | | |
|---|---|---|---|---|---|
| mM | r1 | r2 | r3 | avg | st_dev |

MCF-7 cells

| | | | | | | |
|---|---|---|---|---|---|---|
| HPaCD | 0 | 84.00 | 88.00 | 91.00 | 87.67 | 3.51 |
| | 1 | 35.00 | 41.00 | 48.00 | 41.33 | 6.51 |
| | 2 | 40.00 | 30.00 | 38.00 | 36.00 | 5.29 |
| | 4 | 28.00 | 22.00 | 36.00 | 28.67 | 7.02 |
| HPbCD | 0 | 93.00 | 85.00 | 89.00 | 89.00 | 4.00 |
| | 1 | 68.00 | 74.00 | 78.00 | 73.33 | 5.03 |
| | 2 | 41.00 | 48.00 | 56.00 | 48.33 | 7.51 |
| | 4 | 32.00 | 28.00 | 41.00 | 33.67 | 6.66 |

MDA-MB-231 cells

| | | | | | | |
|---|---|---|---|---|---|---|
| HPaCD | 0 | 95.00 | 91.00 | 99.00 | 95.00 | 4.00 |
| | 1 | 47.00 | 53.00 | 59.00 | 53.00 | 6.00 |
| | 2 | 40.00 | 33.00 | 47.00 | 40.00 | 7.00 |
| | 4 | 38.00 | 32.00 | 25.00 | 31.67 | 6.51 |
| HPbCD | 0 | 92.00 | 98.00 | 100.00 | 96.67 | 4.16 |
| | 1 | 62.00 | 68.00 | 73.00 | 67.67 | 5.51 |
| | 2 | 48.00 | 59.00 | 61.00 | 56.00 | 7.00 |
| | 4 | 48.00 | 44.00 | 25.00 | 39.00 | 12.29 |

Avg: average wound closure;
st_dev: standard deviation.
Results for equivalent doses (1 mM HPaCD, 2 mM HPbCD) are being underlined.

The results are highly consistent:
In all cases, migration decreased with higher concentration of either HPaCD or HPbCD.
In all cases, HPaCD had a stronger effect on migration than HPbCD; Inhibition at 1 mM HPaCD was superior to inhibition at 2 mM HPbCD.

### Discussion

The results in human two human tumor cancer cell lines confirm the hypothesis that the primary effect of bCDs on cell migration is not by scavenging cholesterol, but by scavenging phospholipids. The present invention has shown that “derailed endocytosis” is caused by genetic risk factors causing excessive influx of glycophospholipids, including PC, into the PI cycle FIG. 7. Hence, the previous studies with bCDs, which did not proceed to clinical trials because of risk of cholesterol-related ototoxicity, can now be resumed after the elimination of cholesterol is avoided, while the phospholipid-related efficacy seen in bCD is increased.

HPaCD has the same toxicity as HPbCD (Roka et al. 2015; Huang et al. 2013), but is about twice as effective in scavenging phospholipids, in general, and even more effective in scavenging PC (Monnaert et al. 2004). In children with NPC (Table 3; Matsuo et al. 2013), doses of up to 1000 mg/kg/d HP-β-CD were well tolerated. With 700 mg/kg/d HPbCD having been proven as safe in parenteral treatment of NPC, 700 mg/kg/d HPaCD can now be used to continue with the human experiments that were stopped when bCDs were shown to have cholesterol-related ototoxicity. From the results in FIG. 7, 700 mg/kg/d of αCD have the same efficacy in preventing migration of cancer cells as 2000 mg/kg/d bCD.

### Example 5: Study Design for Testing the Efficacy of a Cyclodextrin in Breast Cancer

Because women with breast cancer have a definitive diagnosis, clinical trials of HPaCD against “derailed endocytosis” should start in this population. As a non-limiting example, a seamless phase 2b/3 clinical trial for HP-α-CD for the prevention of metastases in breast cancer could be conducted in a population of women with:
triple negative breast cancer (tamoxifen and herceptin do not work well in this population) and
axillary lymph node metastasis (patients have established the tendency to develop metastases)

The phase 2b part of the seamless design would have futility as an outcome after the first 80 patients have been seen for at least 2 years and continue to be treated without unblinding, so that they can contribute to the primary endpoint, which would have time to distant metastasis as the outcome. As the recurrence rate is high for the first three years only data will be collected for up to 5 yrs (Kim et al. 2015). Some of the later recruited patients will be administratively censored when the last patient will have been seen for two years. Gehan's test will be used to compare treated vs placebo patients (Gehan 1965).

At an effective median observation time of three years, one would expect 30% of women to have a distant recurrence and could detect a reduction in incidence by 50% (to 15%) with the standard 80% power at the 5% level with 125 subjects per group. The placebo-controlled treatment would be given on top of the standard of care (chemotherapy, radiation, etc.).

As long-term parenteral administration of HP-β-CD (200 mg/kg) was reported to decrease bone mineral density (BMD) in rats (Kantner et al. 2012), the study should carefully monitor bone density.

Human erythrocytes tolerate α-CD better then β-CD (Prakasch et al. 2001). From animal studies, which have less tolerance for cyclodextrin than humans, the dose-limiting factors that are likely are nephrotoxicity and hemolysis.

In animal studies, α-CD did not show ototoxicity (Davidson et al. 2016), still, the dose-finding studies should carefully screen for ototoxicity.

The present invention is also further described by the following claims.

The entire disclosure of each document cited (including patents, patent applications, journal articles, abstracts, laboratory manuals, books, or other disclosures) in the Background of the Invention, Detailed Description, and Examples is hereby incorporated herein by reference in their entireties.

### REFERENCES

1. Vance J E, Karten B (2014) Niemann-Pick C disease and mobilization of lysosomal cholesterol by cyclodextrin. Journal of Lipid Research 55: 1609-21
2. Mohammad N, Malvi P, Meena A S, Singh S V, Chaube B et al. (2014) Cholesterol depletion by methyl-beta-cyclodextrin augments tamoxifen induced cell death by enhancing its uptake in melanoma. Mol Cancer 13: 204
3. Yokoo M, Kubota Y, Motoyama K, Higashi T, Taniyoshi M et al. (2015) 2-Hydroxypropyl-beta-Cyclodextrin Acts as a Novel Anticancer Agent. PLoS One 10: e0141946
4. Song W, Wang F, Lotfi P, Sardiello M, Segatori L (2014) 2-Hydroxypropyl-beta-cyclodextrin promotes transcription factor EB-mediated activation of autophagy: implications for therapy. J Biol Chem 289: 10211-22
5. Vecsernyes M, Fenyvesi F, Bacskay I, Deli M A, Szente L et al. (2014) Cyclodextrins, blood-brain barrier, and treatment of neurological diseases. Arch Med Res 45: 711-29
6. Vance J E (2012) Dysregulation of cholesterol balance in the brain: contribution to neurodegenerative diseases. Disease Models and Mechanisms 5: 746-55
7. Mellman I, Yarden Y (2013) Endocytosis and Cancer. Cold Spring Harbor Perspectives in Biology 5
8. van Spronsen M, Hoogenraad C C (2010) Synapse Pathology in Psychiatric and Neurologic Disease. Current Neurology and Neuroscience Reports 10: 207-14
9. Schreij A M, Fon E A, McPherson P S (2015) Endocytic membrane trafficking and neurodegenerative disease. Cell Mol Life Sci
10. Pandey P, Sliker B, Peters H L, Tuli A, Herskovitz J et al. (2016) Amyloid precursor protein and amyloid precursor-like protein 2 in cancer. Oncotarget: 10430-44
11. Lim S, Yoo B K, Kim H S, Gilmore H L, Lee Y et al. (2014) Amyloid-beta precursor protein promotes cell proliferation and motility of advanced breast cancer. BMC Cancer 14: 928
12. Runowicz C D, Leach C R, Henry N L, Henry K S, Mackey H T et al. (2016) American Cancer Society/American Society of Clinical Oncology Breast Cancer Survivorship Care Guideline. J Clin Oncol 34: 611-35
13. American Cancer Society (2015) Cancer Facts & Figures. 2015. Atlanta: American Cancer Society
14. Risbridger G P, Davis I D, Birrell S N, Tilley W D (2010) Breast and prostate cancer: more similar than different. Nat Rev Cancer 10: 205-12
15. Beebe-Dimmer J L, Yee C, Cote M L, Petrucelli N, Palmer N et al. (2015) Familial clustering of breast and prostate cancer and risk of postmenopausal breast cancer in the Women's Health Initiative Study. Cancer 121: 1265-72
16. lacopetta D, Rechoum Y, Fuqua S A W (2012) The Role of Androgen Receptor in Breast Cancer. Drug discovery today Disease mechanisms 9: e19-e27
17. Castro E, Eeles R (2012) The role of BRCA1 and BRCA2 in prostate cancer. Asian J Androl 14: 409-14
18. Finn R S, Aleshin A, Slamon D J (2016) Targeting the cyclin-dependent kinases (CDK) 4/6 in estrogen receptor-positive breast cancers. Breast Cancer Res 18: 17
19. Bosch A, Li Z, Bergamaschi A, Ellis H, Toska E et al. (2015) PI3K inhibition results in enhanced estrogen receptor function and dependence in hormone receptor-positive breast cancer. Sci Transl Med 7: 283ra51-ra51
20. Takahashi S, Homma K, Zhou Y, Nishimura S, Duan C et al. (2016) Susceptibility of outer hair cells to cholesterol chelator 2-hydroxypropyl-beta-cyclodextrine is prestin-dependent. Sci Rep 6: 21973
21. Cronin S, Lin A, Thompson K, Hoenerhoff M, Duncan R K (2015) Hearing Loss and Otopathology Following Systemic and Intracerebroventricular Delivery of 2-Hydroxypropyl-Beta-Cyclodextrin. J Assoc Res Otolaryngol 16: 599-611
22. Liu X-M, Wiswall A T, Rutledge J E, Akhter M P, Cullen D M et al. (2008) Osteotropic β-cyclodextrin for local bone regeneration. Biomaterials 29: 1686-92
23. Schmid S L, Mettlen M (2013) Cell biology: Lipid switches and traffic control. Nature 499: 161-2
24. Bohdanowicz M, Grinstein S (2013) Role of phospholipids in endocytosis, phagocytosis, and macropinocytosis. Physiol Rev 93: 69-106
25. Hesketh G G, Perez-Dorado I, Jackson L P, Wartosch L, Schafer I B et al. (2014) VARP is recruited on to endosomes by direct interaction with retromer, where together they function in export to the cell surface. Dev Cell 29: 591-606
26. Mosesson Y, Mills G B, Yarden Y (2008) Derailed endocytosis: an emerging feature of cancer. Nat Rev Cancer 8: 835-50
27. Kocherlakota C, Mogadati P S, Mandavilli S R (2009) Complexes comprising zoledronic acid and cyclodextrins. U.S., U.S. Ser. No. 12/173,871
28. Clear K J, Smith B D (2015) Synthetic Receptors for polar Lipids: Design Principles and Applications. In: Smith, B D (ed) Monographs in Supramolecular Chemistry. (14): Royal Society of Chemistry, 404-36
29. Gimpl G, Gehrig-Burger K (2011) Probes for studying cholesterol binding and cell biology. Steroids 76: 216-31
30. shityakov S, Salmas R E, Salvador E, Roewer N, Broscheit J et al. (2016) Evaluation of the potential toxicity of unmodified and modified cyclodextrins on murine blood-brain barrier endothelial cells. J Toxicol Sci 41: 175-84
31. Hipler U C, Schonfelder U, Hipler C, Elsner P (2007) Influence of cyclodextrins on the proliferation of HaCaT keratinocytes in vitro. J Biomed Mater Res A 83: 70-9
32. Ohtani Y, Irie T, Uekama K, Fukunaga K, Pitha J (1989) Differential effects of α-, β- and γ-cyclodextrins on human erythrocytes. European Journal of Biochemistry 186: 17-22
33. Chen X, Hui L, Soliman M L, Geiger J D (2014) Altered Cholesterol Intracellular Trafficking and the Development of Pathological Hallmarks of Sporadic A D. J Parkinsons Dis Alzheimers Dis 1
34. Wittkowski K M, Sonakya V, Bigio B, Tonn M K, Shic F et al. (2014) A novel computational biostatistics approach implies impaired dephosphorylation of growth factor receptors as associated with severity of autism. Transl Psychiatry 4: e354
35. Waugh M G (2015) PIPs in neurological diseases. Biochim Biophys Acta 1851: 1066-82
36. Prakasch A S, Abbott P J (2001) Safety evaluation of certain food additives and contaminants: alpha-cyclodextrin. WHO/JECFA Food Additive Series 48: 1030
37. Frank D W, Gray J E, Weaver R N (1976) Cyclodextrin nephrosis in the rat. Am J Pathol 83: 367-82
38. Pitha J, Szente L (1982) Cyclodextrins and Congeners in Parenteral Applications. In: Proceedings of the First International Symposium on Cyclodextrins. Springer Netherlands, 457-66
39. Irie T, Tsunenari Y, Uekama K, Pitha J (1988) Effect of bile on the intestinal absorption of α-cyclodextrin in rats. International Journal of Pharmaceutics 43: 41-4
40. Ooms Lisa M, Binge Lauren C, Davies Elizabeth M, Rahman P, Conway James R W et al. (2015) The Inositol Polyphosphate 5-Phosphatase PIPP Regulates AKT1-Dependent Breast Cancer Growth and Metastasis. Cancer cell 28: 155-69
41. Ma K, Cheung S M, Marshall A J, Duronio V (2008) PI(3,4,5)P3 and PI(3,4)P2 levels correlate with PKB/akt phosphorylation at Thr308 and Ser473, respectively; PI(3,4)P2 levels determine PKB activity. Cell Signal 20: 684-94
42. Majerus P W, York J D (2009) Phosphoinositide phosphatases and disease. Journal of Lipid Research 50: S249-S54
43. Woolley J F, Dzneladze I, Salmena L (2015) Phosphoinositide signaling in cancer: INPP4B Akt(s) out. Trends Mol Med 21: 530-2
44. Chew C L, Chen M, Pandolfi P P (2016) Endosome and INPP4B. Oncotarget 7: 5-6
45. Mayer I A, Arteaga C L (2016) The PI3K/AKT Pathway as a Target for Cancer Treatment. Annu Rev Med 67: 11-28
46. Irie T, Uekama K (1997) Pharmaceutical applications of cyclodextrins. III. Toxicological issues and safety evaluation. J Pharm Sci 86: 147-62
47. Loftsson T, Brewster M E (1996) Pharmaceutical applications of cyclodextrins. 1. Drug solubilization and stabilization. J Pharm Sci 85: 1017-25
48. Stella V J, He Q (2008) Cyclodextrins. Toxicol Pathol 36: 30-42
49. Szejtli J, Gerloczy A, Sebestyen G, Fonagy A (1981) Influencing of resorption and side-effects of salicylic acid by complexing with beta-cyclodextrin. Pharmazie 36: 283-6
50. Kanai A, Alba R M, Takano T, Kobayashi C, Nakajima A et al. (1989) The effect on the cornea of alpha cyclodextrin vehicle for cyclosporin eye drops. Transplant Proc 21: 3150-2
51. Brewster M E, Loftsson T (2007) Cyclodextrins as pharmaccutical solubilizers. Advanced Drug Delivery Reviews 59: 645-66
52. Monnaert V, Tilloy S, Bricout H, Fenart L, Cecchelli R et al. (2004) Behavior of α-, β-, and γ-Cyclodextrins and Their Derivatives on an in Vitro Model of Blood-Brain Barrier. Journal of Pharmacology and Experimental Therapeutics 310: 745-51
53. Davis M E, Brewster M E (2004) Cyclodextrin-based pharmaceutics: past, present and future. Nat Rev Drug Discov 3: 1023-35
54. Loftsson T, Brewster M E (2010) Pharmaceutical applications of cyclodextrins: basic science and product development. J Pharm Pharmacol 62: 1607-21
55. Loftsson T, Moya-Ortega M D, Alvarez-Lorenzo C, Concheiro A (2016) Pharmacokinetics of cyclodextrins and drugs after oral and parenteral administration of drug/cyclodextrin complexes. J Pharm Pharmacol 68: 544-55
56. Gould S, Scott R C (2005) 2-Hydroxypropyl-beta-cyclodextrin (HP-beta-CD): a toxicology review. Food Chem Toxicol 43: 1451-9
57. Irie T, Otagiri M, Sunada M, Uekama K, Ohtani Y et al. (1982) Cyclodextrin-induced hemolysis and shape changes of human erythrocytes in vitro. J Pharmacobiodyn 5: 741-4
58. Roka E, Ujhelyi Z, Deli M, Bocsik A, Fenyvesi E et al. (2015) Evaluation of the Cytotoxicity of alpha-Cyclodextrin Derivatives on the Caco-2 Cell Line and Human Erythrocytes. Molecules 20: 20269-85
59. Ono N, Arima H, Hirayama F, Uekama K (2001) A moderate interaction of maltosyl-alpha-cyclodextrin with Caco-2 cells in comparison with the parent cyclodextrin. Biol Pharm Bull 24: 395-402
60. NIH (2015) Study of 2-hydroxypropyl-β-cyclodextrin (VTS-270) to Treat Niemann-Pick Type C1 (NPC1) Disease. ClinicalTrialsgov: NCT02534844
61. Fauvelle F, Debouzy J C, Nardin R, Gadelle A (1994) Nuclear magnetic resonance study of a polar headgroup determined α-cyclodextrin-phospholipid association. Bioelectrochemistry and Bioenergetics 33: 95-9
62. Fauvelle F, Debouzy J C, Crouzy S, Goschl M, Chapron Y (1997) Mechanism of alpha-cyclodextrin-induced hemolysis. 1. The two-step extraction of phosphatidylinositol from the membrane. J Pharm Sci 86: 935-43
63. Huang Z, London E (2013) Effect of cyclodextrin and membrane lipid structure upon cyclodextrin-lipid interaction. Langmuir 29: 14631-8
64. Kamar R I, Organ-Darling L E, Raphael R M (2012) Membrane Cholesterol Strongly Influences Confined Diffusion of Prestin. Biophysical Journal 103: 1627-36
65. Kantner I, Erben R G (2012) Long-term parenteral administration of 2-hydroxypropyl-beta-cyclodextrin causes bone loss. Toxicol Pathol 40: 742-50
66. Ward S, O'Donnell P, Fernandez S, Vite C H (2010) 2-hydroxypropyl-beta-cyclodextrin raises hearing threshold in normal cats and in cats with Niemann-Pick type C disease. Pediatr Res 68: 52-6
67. Maarup T J, Chen A H, Porter F D, Farhat N Y, Ory D S et al. (2015) Intrathecal 2-hydroxypropyl-beta-cyclodextrin in a single patient with Niemann-Pick C1. Mol Genet Metab 116: 75-9
68. Wittkowski K M, Sonakya V, Song T, Seybold M P, Keddache M et al. (2013) From single-SNP to wide-locus: genome-wide association studies identifying functionally related genes and intragenic regions in small sample studies. Pharmacogenomics 14: 391-401
69. Hunter D J, Kraft P, Jacobs K B, Cox D G, Yeager M et al. (2007) A genome-wide association study identifies alleles in FGFR2 associated with risk of sporadic postmenopausal breast cancer. Nature Genet 39: 870-4
70. Garcia-Closas M, Couch F J, Lindstrom S, Michailidou K, Schmidt M K et al. (2013) Genome-wide association studies identify four ER negative-specific breast cancer risk loci. Nat Genet 45: 392-8, 8e1-2
71. Mann H B, Whitney D R (1947) On a test of whether one of two random variables is stochastically larger than the other. Ann Math Stat 18: 50-60
72. Wilcoxon F (1954) Individual comparisons by ranking methods. Biometrics 1: 80-3
73. Armitage P (1955) Tests for linear trends in proportions and frequencies. Biometrics 11: 375-86
74. Hoeffding W (1948) A class of statistics with asymptotically normal distribution. Ann Math Stat 19: 293-325
75. Pearson T A, Manolio T A (2008) How to interpret a genome-wide association study. JAMA 299: 1335-44
76. Cleveland W S, Devlin S J (1988) Locally Weighted Regression: An Approach to Regression Analysis by Local Fitting. J Am Statist Assoc 83: 596-610
77. Tukey J W (1980) We Need Both Exploratory and Confirmatory. American Statistician 34: 23-5
78. Cicek M S, Cunningham J M, Fridley B L, Serie D J, Bamlet W R et al. (2012) Colorectal cancer linkage on chromosomes 4q21, 8q13, 12q24, and 15q22. PLoS One 7: e38175
79. Shulga Y V, Myers D S, Ivanova P T, Milne S B, Brown H A et al. (2010) Molecular species of phosphatidylinositol-cycle intermediates in the endoplasmic reticulum and plasma membrane. Biochemistry 49: 312-7
80. Busa W B (1988) Roles for the phosphatidylinositol cycle in early development. Philos Trans R Soc Lond B Biol Sci 320: 415-26
81. Kerr W G (2011) Inhibitor and activator: dual functions for SHIP in immunity and cancer. Ann N Y Acad Sci 1217: 1-17
82. Sengelaub C A, Navrazhina K, Ross J B, Halberg N, Tavazoie S F (2015) PTPRN2 and PLCβ1 promote metastatic breast cancer cell migration through PI(4,5)P2-dependent actin remodeling. The EMBO Journal
83. Rocks N, Bekaert S, Coia I, Paulissen G, Gueders M et al. (2012) Curcumin-cyclodextrin complexes potentiate gemcitabine effects in an orthotopic mouse model of lung cancer. Br J Cancer 107: 1083-92
84. Agardan N B, Degim Z, Yilmaz S, Altintas L, Topal T (2015) The Effectiveness of Raloxifene-Loaded Liposomes and Cochleates in Breast Cancer Therapy. AAPS PharmSciTech
85. Rajendran L, Knolker H-J, Simons K (2010) Subcellular targeting strategies for drug design and delivery. Nat Rev Drug Discov 9: 29-42
86. Ben Halima S, Mishra S, Raja K M, Willem M, Baici A et al. (2016) Specific Inhibition of beta-Secretase Processing of the Alzheimer Disease Amyloid Precursor Protein. Cell Rep 14: 2127-41
87. Rodal S K, Skretting G, Garred └, Vilhardt F, van Deurs B et al. (1999) Extraction of Cholesterol with Methyl-β-Cyclodextrin Perturbs Formation of Clathrin-coated Endocytic Vesicles. Molecular Biology of the Cell 10: 961-74
88. Le Tourneau C, Lee J J, Siu L L (2009) Dose Escalation Methods in Phase I Cancer Clinical Trials. JNCI Journal of the National Cancer Institute 101: 708-20
89. Kim H J, Ahn S G, Lee H M, Park J T, Han K et al. (2015) Metastasis-Free Interval Is Closely Related to Tumor Characteristics and Has Prognostic Value in Breast Cancer Patients with Distant Relapse. J Breast Cancer 18: 371-7
90. Gehan E A (1965) A generalised Wilcoxon test for comparing arbitrarily singly censored samples. Biometrika 52: 203-23

1. A method of treatment and/or prevention of cancer in a subject in need of treatment comprising administering to the subject in need thereof a therapeutically effective amount of cyclodextrin, a derivative thereof, or a pharmaceutically acceptable salt thereof.

2. The method of claim 1, wherein the cancer comprises a cancer cell that is derived from or is an epithelial cell.

3. The method of claim 1, wherein the cancer comprises a cancer cell that has a dysfunctional PI cycle.

4. The method of claim 1, wherein the cyclodextrin is α-cyclodextrin a derivative thereof, or a pharmaceutically acceptable salt thereof.

5. The method of claim 1, wherein the cyclodextrin is 2-Hydroxypropyl-α-cyclodextrin (HP-α-CD) or a pharmaceutically acceptable salt thereof.

6. The method of claim 1, wherein the therapeutically effective amount is at a concentration of from about 0.1 mM to about 1.25 mM within a dosage form.

7.-8. (canceled)

9. The method of claim 1, wherein the cancer comprises one or a plurality of cancer cells that have dysfunction of the PI cycle, thereby resulting in a failure to properly metabolize phospholipids.

10. The method of claim 1, wherein the cancer comprises cancer cells that have a high rate of endocytosis and/or exocytosis.

11. A method of treating a subject that comprises cancer cells that have a high rate of endocytosis or exocytosis, the method comprising the steps of:
(i) identifying the subject as an subject who has cancer by imaging, wherein said imaging indicates that the cancer comprises cancer cells that have a high rate of endocytosis and/or exocytosis as compared to a non-cancerous cell; and
(ii) subsequently administering to said subject a therapeutically effective amount of cyclodextrin or a salt thereof, wherein said therapeutically effective amount of said cyclodextrin is sufficient to scavenge phospholipid from the within the cancer cells and/or outside of the cancer cells to result in modulation in the amount phospholipid metabolized by the cancer cell or a reduction in the rate of the PI cycle within the cancer cell.

12. The method of claim 11, wherein the cancer is metastatic cancer.

13. The method of claim 11, wherein the cancer comprises a cancer cell that is derived from or is an epithelial cell.

14.-15. (canceled)

16. The method of claim 11, wherein the cyclodextrin is 2-Hydroxypropyl-β-cyclodextrin (HP-β-CD), a pharmaceutically acceptable salt thereof or 2-Hydroxypropyl-α-cyclodextrin (HP-α-CD) or a pharmaceutically acceptable salt thereof.

17.-18. (canceled)

19. The method of claim 11, wherein the therapeutically effective amount is a dose of from about 1 mg/kg to about 1000 mg/kg.

20. The method of claim 11, wherein the cancer comprises one or a plurality of cancer cells that have dysfunctional PI cycle, thereby resulting in a failure to properly metabolize phospholipids.

21. The method of claim 1, wherein the step of administering is accomplished by subcutaneous injection, intravenous injection, intramuscular injection, intrathecal injection, intraperitoneal injection, transdermal administration, transmucosal administration, oral administration or pulmonary administration.

22. (canceled)

23. The method of claim 1, wherein the cancer is prostate cancer, breast cancer, or colon cancer.

24.-36. (canceled)

37. A method of treating and/or preventing a neurodegenerative disease in a subject in need thereof, the method comprising administering to the subject in need thereof a therapeutically effective amount of cyclodextrin, a derivative thereof, or a pharmaceutically acceptable salt thereof.

38. The method of claim 37, wherein said subject comprises a central nervous system tissue comprising one or a plurality of cells that have a dysfunctional PI cycle and wherein the therapeutically effective amount of said cyclodextrin is sufficient to scavenge phospholipid from within the central nervous system tissue of the subject to result in reduction in the amount phospholipid metabolized by the cell or a modulation in the rate of the PI cycle within the cell.

39. (canceled)

40. The method of claim 37, wherein the cyclodextrin is 2-Hydroxypropyl-α-cyclodextrin (HP-α-CD) or a pharmaceutically acceptable salt thereof.

41.-42. (canceled)

43. The method of claim 37, wherein the therapeutically effective amount is a dose of from about 1 mg/kg to about 1000 mg/kg.

44.-45. (canceled)

46. The method of claim 37, wherein the neurodegenerative disease is Alzheimer's Disease or Parkinson's Disease.

47.-56. (canceled)
